SEQUENCE LISTING (8 sequences)

SEQ ID NO: 1 - RNA, 11 nt, synthetic construct
  source 1..11: mol_type = other RNA
gggatcctacc

SEQ ID NO: 2 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 3 - RNA, 100 nt, synthetic construct
  source 1..100: mol_type = other RNA
cgggtggcatccctgtgacccctccccagtgcctctcctggccctggaagttgccactccagtgcccaccagccttgtcctaataaaattaagttgcatc

SEQ ID NO: 4 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 5 - RNA, 1941 nt, synthetic construct
  source 1..1941: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaagccatccttgttgtcatgctgtacacattcaccaccgcaaatgcggataccctgtgtatcggctaccacgcaaataattccaccgacaccgttgataccgtcctggaaaagaacgtgacagtgactcacagcgtcaatctccttgaggataaacataatggcaagctgtgcaagctgagaggcgtggctcccctgcatctgggaaagtgcaacatcgctggttggatcctcgggaacccagagtgtgagtccctctcaaccgcacggtcttggtcatacatcgtggagactagcaattcagacaacggcacatgctaccccggtgacttcattaactacgaggagctgagagaacagctgagttccgtgtcatccttcgagagattcgaaatcttccccaaaacctcctcctggcccaatcatgactccgacaatggagtgacagccgcttgtccccacgccggtgccaagagtttctataagaacctcatctggctggtgaaaaagggcaagtcctatcccaaaattaaccagacctacattaacgataaggggaaagaagtcctggtcctgtgggggatacaccacccccctaccatcgccgaccagcagtctctgtatcagaacgccgacgcctacgtgttcgtgggtaccagccgttatagtaaaaagttcaagccagaaattgccaccagacctaaggtgcgcgaccaggagggccgcatgaactactactggaccctggtggaacctggcgacaagattacattcgaggccactgggaacctggtggcacccagatacgcctttacaatggaacgggatgctgggagcggaatcattatctccgatacccctgtccacgactgcaatactacctgtcagaccccagaaggcgctatcaatacctctctgcctttccaaaacgtgcaccctatcactatcgggaaatgtcccaagtatgtgaaaagcaccaaactgcgcctggcaaccggtctgagaaatgtgccctccatccagtcccgcggcttgttcggtgcaatcgctggctttatcgagggtggctggactggaatggtcgatggctggtacggctaccatcaccagaacgagcaggggtccgggtatgctgccgacctgaaaagcactcagaacgccatcgataaaatcactaacaaggtgaactccgtgatcgaaaagatgaatacacagttcacagcagttggcaaggagttcaaccacctggaaaaacggatagagaacctgaataagaaagtcgatgatggctttctggacatctggacttacaatgccgagctgctggtgctcctggaaaacgagcggacactggattatcacgactcaaacgtgaagaacctgtatgaaaaggtgcgtaaccagctgaaaaacaacgccaaggaaatcggcaatggctgtttcgaattttaccacaagtgtgataatacctgtatggagagcgttaagaacgggacttacgactacccaaaatacagcgaggaggccaagctgaaccgggagaagatcgacggcgtcaaactcgactccactagaatataccagattctcgccatctatagcacagtggcatcaagtctcgtcctggtggtgtcactgggagccatcagcttttggatgtgcagcaatggatccctccagtgtaggatctgcatctaacgggtggcatccctgtgacccctccccagtgcctctcctggccctggaagttgccactccagtgcccaccagccttgtcctaataaaattaagttgcatc

SEQ ID NO: 6 - RNA, 1941 nt, synthetic construct
  source 1..1941: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaagaccatcatcgctctgtcctacatcctgtgcctggtgtttgctcagaaaatccccgggaatgacaattccactgccactctctgcctgggccatcatgccgtgccaaatggaaccattgtcaagactataacaaatgaccgcatcgaagtgaccaacgctaccgagctggttcagaacagcagtattggagaaatctgcgattccccacaccagatactggatggcggcaactgcaccctgatcgacgcactgctgggtgaccctcagtgcgacggatttcagaataaggagtgggaccttttcgttgagcgcagcagagccaatagcaactgctacccgtacgacgtgccggattacgccagtcttcgaagcctggtcgcatccagcgggacactggagtttaagaatgagtcctttaattggacaggcgtgaagcagaacgggactagcagcgcatgcattcggggcagtagctcatccttctttagccgactgaactggctgacccacctcaactacacataccccgcactgaatgtgactatgccaaacaaagaacagtttgacaaactgtacatctggggagtgcaccatcctagcacagacaaggaccagatcagcctgtttgcccagcccagcggcaggattaccgtgtccacaaaacggtcacagcaagccgtgatccctaatattggatcccgcccccggataagggacatccctagtcgcatcagtatctactggaccatcgtgaagcccggagatatcttgctcatcaatagcactggcaacctcattgcccccaggggctattttaagatcagaagcggcaagtccagcattatgcgcagcgacgcacccattggcaagtgcaagtccgagtgcatcactcctaatgggtccatcccaaacgacaagccattccaaaatgtcaacagaatcacctacggggcttgcccccgctacgtgaagcagagtacactgaaactggccaccgggatgcgcaacgtgcccgagaagcaaactagaggcatctttggagctatcgctggcttcattgagaatggctgggagggtatggtggacggctggtacggattccgccaccagaatagcgaaggcagaggccaggcagcagacttgaagtccacccaggccgccattgatcagatcaacggcaaactgaatcggcttattggaaaaacaaacgagaagttccatcagattgagaaggagtttagcgaggtggagggccgcgtgcaggatctggaaaagtacgttgaagacaccaagatcgacctgtggtcatacaatgcagagctgctcgttgccctggaaaatcagcacacaattgaccttacagactccgaaatgaataagctctttgaaaagaccaagaagcagctgcgcgagaacgccgaggatatggggaacggttgttttaagatctaccacaagtgtgacaacgcctgcattgggtccatccgaaatgaaacatacgaccacaacgtgtatagagatgaggccctgaacaaccgattccagattaagggagtcgagctgaagagtggctataaggactggatcctgtggatctcattcgccatgtcatgcttccttctgtgtattgctctgctcggcttcatcatgtgggcttgccagaaaggcaatatccggtgcaacatctgcatctaacgggtggcatccctgtgacccctccccagtgcctctcctggccctggaagttgccactccagtgcccaccagccttgtcctaataaaattaagttgcatc

SEQ ID NO: 7 - RNA, 1989 nt, synthetic construct
  source 1..1989: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaagcaatcatagtgctgctgatggtggtgactagcaatgccgatcggatctgcaccggcatcacttccagtaacagccctcatgtggtcaaaaccgccacacagggcgaggtgaacgtgaccggagtgattccactgacaactacaccaacgaagagtcacttcgccaacctgaagggcaccgaaacacgaggcaagctctgccccaagtgtctgaattgcaccgacctggacgtcgctttgggccgccctaaatgtaccggcaaaataccttccgccagagtgtccatcctgcacgaggtgcgccccgtgacctccgggtgttttcccataatgcacgaccgcactaaaatccgccagctgcccaatcttctgagggggtacgaacatgtcaggctgtccactcacaacgtgatcaacgcagaagacgcccccggaaggccttatgagattggaaccagtgggtcctgcccaaacattaccaacggcaacggcttcttcgccactatggcctgggccgtgccaaagaacaagaccgccaccaaccccctgacaattgaagtcccttacatctgcacagagggagaggatcagatcaccgtgtgggggtttcactctgataacgaaactcagatggccaagctgtacggggattctaaaccccagaagttcaccagtagcgctaacggggtgaccacccattatgtgtctcagatcggaggtttcccaaatcagaccgaggacggcggactgccccagtctggaaggatcgtagtggactatatggtgcagaagagtggaaaaaccggcaccattacctatcagcgcggcatactgctgccacagaaggtgtggtgtgcttccggcaggtccaaggttatcaaagggtccctccccctgatcggcgaagcagattgtctgcacgagaagtacggcggactgaataagagcaaaccctactacaccggagaacacgctaaggcaattgggaattgtccgatctgggtgaagacgcccctgaaactggccaatggcacaaaataccggccccccgctaagctgctgaaggaacgggggttcttcggcgccatagccggctttctggagggaggctgggagggcatgatagccgggtggcacggctacacttcccatggggctcacggggtggctgtggccgccgacctgaagtctacgcaggaagctatcaacaaaatcactaagaacctgaacagcctgtcggaattggaggtcaagaatctgcagcggctgagcggcgccatggatgagctgcacaatgagatcctggagcttgacgagaaggtcgatgatcttcgggccgatacaattagtagccaaattgagttggccgtgctgctcagcaacgaaggcataatcaacagcgaggacgagcacctcctggctctggagagaaagctgaagaagatgctcggccctagcgcagttgagatcggaaacggctgcttcgaaaccaagcacaagtgcaaccagacctgcctggacaggatcgcggcaggaacattcgacgctggggaattcagcctccccaccttcgacagcctgaacatcacagccgccagtctgaatgatgacggactggataaccataccatcctgctgtactactctaccgctgcttcctccctggccgtgacattgatgatcgcaatctttgtggtttatatggtgagccgagacaacgtcagttgcagtatctgcctttaacgggtggcatccctgtgacccctccccagtgcctctcctggccctggaagttgccactccagtgcccaccagccttgtcctaataaaattaagttgcatc

SEQ ID NO: 8 - RNA, 1995 nt, synthetic construct
  source 1..1995: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatgaaagccatcattgtgctgctgatggttgttacaagcaacgccgaccgcatctgcaccgggattacaagcagcaatagccctcacgtggtgaagacagcaacacagggagaggtgaacgtgaccggcgtgattccactgacaaccaccccaactaaatcttactttgcaaacctgaaagggacacggaccagaggaaagctgtgccctgattgcctgaattgcacagacctggacgtggccctgggcagaccaatgtgcgtgggcactacaccaagcgccaaggcctccatcctccatgaggtgcggcccgtgacttctggatgtttccccattatgcacgacagaaccaagattagacagctgccaaacctgctccgcggctacgagaaaattcgcctgtctacacagaatgtgatcgacgccgagaaggctccaggaggaccatacagactggggacttctggcagctgccctaacgccacctctaagatcgggttcttcgcaaccatggcttgggccgtgcctaaagacaattacaagaatgccaccaatccactgactgtcgaggtgccatatatttgcacagagggggaggaccagatcactgtgtggggctttcatagcgataataagactcagatgaagtctctctacggcgactctaaccctcagaagttcacctcctctgccaacggggtgacaacacactacgtgtcccagatcggggactttcctgaccagaccgaggatggaggactgcctcagtctggacgcatcgtggtggactatatgatgcagaagcctgggaagaccggcactatcgtgtaccagaggggcgtgctgctgccccaaaaggtgtggtgtgcctccggaagaagcaaagtgattaagggatccctgcctctgattggggaggccgattgcctgcatgaagagtatggagggctgaacaagtccaagccatactatacaggaaagcacgcaaaagccatcggcaactgtcccatctgggtcaaaactcctctgaagctggccaacggcaccaaataccgccctccagccaagctgctgaaagaacgcggattcttcggcgccattgcagggtttctggaaggaggctgggagggcatgattgctggatggcacggatatacctctcacggcgctcacggggtggccgtggccgccgatctgaagtccacacaggaggcaattaacaagatcaccaagaatctgaattcactgtccgagctcgaagtgaaaaacctgcagcgcctgtccggcgccatggacgagctgcacaatgaaatcctggagctggacgagaaggtggacgacctgcgggctgacactatcagcagccagatcgagctggcagtgctgctgagcaatgagggcatcatcaactcagaagacgaacacctcctggcactggaaaggaaactcaagaagatgctgggcccctccgcagtggacattgggaacggctgtttcgaaaccaagcataagtgtaaccagacttgtctggataggatcgcagcaggaaccttcaacgccggcgaattttctctgccaacatttgactccctgaacatcacagctgcatccctgaacgacgacggactggacaatcacaccatcctgctgtactactctactgccgctagctccctggccgtgaccctgatgctggccatcttcatcgtgtacatggtttccagggataacgtgtcttgtagcatttgcctgtaacgggtggcatccctgtgacccctccccagtgcctctcctggccctggaagttgccactccagtgcccaccagccttgtcctaataaaattaagttgcatc

(19) United States
(12) Patent Application Publication
(10) Pub. No.: US 2026/0199449 A1
(43) Pub. Date: Jul. 16, 2026

(54) MULTIVALENT INFLUENZA MRNA VACCINES

(71) Applicant: SANOFI PASTEUR INC., Swiftwater, PA (US)

(72) Inventors: Timothy ALEFANTIS, Cambridge, MA (US); Maryann GIEL-MOLONEY, Cambridge, MA (US); Florence ARVIS, Paris (FR)

(21) Appl. No.: 19/410,181

(22) Filed: Dec. 5, 2025

Related U.S. Application Data
(63) Continuation of application No. PCT/IB2024/000346, filed on Jun. 28, 2024.

(30) Foreign Application Priority Data
Jun. 28, 2023 (EP) 23315259.4

(51) Int. Cl. A61K 39/145; A61K 9/51; A61K 39/00; A61K 47/28; A61K 47/34; A61P 37/04

(52) U.S. Cl. CPC A61K 39/145; A61K 9/5123; A61K 47/28; A61K 47/34; A61P 37/04; A61K 2039/53; A61K 2039/70

(57) ABSTRACT

The present disclosure provides multivalent influenza vaccine compositions comprising at least three messenger RNAs (mRNAs) encoding a combination of influenza A and influenza B hemagglutinin (HA) antigens, wherein the mRNA encoding the HA antigen of the influenza A virus is present in a different ratio (w/w) than the mRNA encoding the influenza B virus, and methods of eliciting an immune response by administering said compositions. In particular, the disclosures relate to mRNA encoding these antigens formulated in a lipid nanoparticle (LNP).

### RELATED APPLICATION

This application is a continuation of International Patent Application No. PCT/IB2024/000346, filed Jun. 28, 2024, which claims priority to European Patent Application Serial No. 23315259.4, filed on Jun. 28, 2023, the disclosures of which are hereby incorporated by reference in their entirety.

### SEQUENCE LISTING

The instant application contains a Sequence Listing which has been submitted electronically in XML format and is hereby incorporated by reference in its entirety. Said XML file, created on Dec. 4, 2025, is named 772521_SA9-380PCCON_ST26.xml and is 16,134 bytes in size.

### BACKGROUND OF THE INVENTION

Messenger RNA (mRNA) based vaccines provide a promising alternative to traditional subunit vaccines, which contain antigenic proteins derived from a pathogen. Antigen proteins are usually recombinantly made and require bacterial fermentation and/or cell culture, as well as complex purification. Vaccines based on mRNA allow de novo expression of complex antigens in the vaccinated subject, which in turn allows proper post-translational modification and presentation of the antigens in its natural conformation. Unlike traditional technologies, the manufacture of mRNA vaccines does not require complex and costly bacterial fermentation, tissue culture, and purification processes. Moreover, once established, the manufacturing process for mRNA vaccines can be used for a variety of antigens, enabling rapid development and deployment of mRNA vaccines. Further, mRNA vaccines are inherently safe delivery vectors as they express the antigens only transiently and do not integrate into the host genome. Because antigens encoded by mRNAs are produced in vivo in the vaccinated individual, mRNA vaccines are especially effective in eliciting both humoral and T cell mediated immunity.

Current mRNA-based multivalent influenza vaccines under investigation encode hemagglutinin (HA) antigens from both the influenza A virus and the B influenza virus in equal ratios (w/w). While these vaccines are immunogenic, ongoing trials indicate that B strain immunogenicity is lower than A strain immunogenicity and immune responses in humans to B strain influenza viruses are sub-optimal with these vaccines. Accordingly, there exists a need for multivalent mRNA-based influenza vaccines with improved B strain immunogenicity.

### SUMMARY OF THE INVENTION

In one aspect, the disclosure provides a composition comprising at least three messenger RNAs (mRNAs), wherein the at least three mRNAs comprise an open reading frame (ORF) encoding a hemagglutinin (HA) antigen selected from the group consisting of: (i) a first mRNA encoding an HA antigen of a first influenza A virus; (ii) a second mRNA encoding an HA antigen of a second influenza A virus, wherein the first influenza A virus and the second influenza A virus are of different subtypes; and (iii) a third mRNA encoding an HA antigen of a first influenza B virus, wherein the mRNA encoding the HA antigen of the influenza A virus is present in a different ratio (w/w) than the mRNA encoding the HA antigen of the influenza B virus.

In certain embodiments, the composition comprises a fourth mRNA encoding an HA antigen of a second influenza B virus, wherein the first influenza B virus and the second influenza B virus are of different lineages.

In certain embodiments, the composition comprises the first mRNA, the second mRNA, and the third mRNA present in the ratio (w/w) of about 1:1:2.

In certain embodiments, the composition comprises the first mRNA, the second mRNA, and the third mRNA present in the ratio (w/w) of about 1:1:3.

In certain embodiments, the composition comprises the first mRNA, the second mRNA, and the third mRNA present in the ratio (w/w) of about 1:1:4.

In certain embodiments, the composition comprises the first mRNA, the second mRNA, and the third mRNA present in the ratio (w/w) of about 1:1:5.

In certain embodiments, the composition comprises the first mRNA, the second mRNA, and the third mRNA present in the ratio (w/w) of about 1:1:6.

In certain embodiments, the composition comprises the first mRNA, the second mRNA, and the third mRNA present in the ratio (w/w) of about 1:1:7.

In certain embodiments, the composition comprises the first mRNA, the second mRNA, and the third mRNA present in the ratio (w/w) of about 1:1:8.

In certain embodiments, the composition comprises the first mRNA, the second mRNA, and the third mRNA present in the ratio (w/w) of about 1:1:9.

In certain embodiments, the composition comprises the first mRNA, the second mRNA, and the third mRNA present in the ratio (w/w) of about 1:1:10.

In certain embodiments, the composition comprises the first mRNA, the second mRNA, and the third mRNA present in the ratio (w/w) of about 1:1:2 to about 1:1:10.

In certain embodiments, the composition comprises the first mRNA, the second mRNA, the third mRNA, and the fourth mRNA present in the ratio (w/w) of about 1:1:2:2.

In certain embodiments, the composition comprises the first mRNA, the second mRNA, the third mRNA, and the fourth mRNA present in the ratio (w/w) of about 1:1:3:3.

In certain embodiments, the composition comprises the first mRNA, the second mRNA, the third mRNA, and the fourth mRNA present in the ratio (w/w) of about 1:1:4:4.

In certain embodiments, the composition comprises the first mRNA, the second mRNA, the third mRNA, and the fourth mRNA present in the ratio (w/w) of about 1:1:5:5.

In certain embodiments, the composition comprises the first mRNA, the second mRNA, the third mRNA, and the fourth mRNA present in the ratio (w/w) of about 1:1:6:6.

In certain embodiments, the composition comprises the first mRNA, the second mRNA, the third mRNA, and the fourth mRNA present in the ratio (w/w) of about 1:1:7:7.

In certain embodiments, the composition comprises the first mRNA, the second mRNA, the third mRNA, and the fourth mRNA present in the ratio (w/w) of about 1:1:8:8.

In certain embodiments, the composition comprises the first mRNA, the second mRNA, the third mRNA, and the fourth mRNA present in the ratio (w/w) of about 1:1:9:9.

In certain embodiments, the composition comprises the first mRNA, the second mRNA, the third mRNA, and the fourth mRNA present in the ratio (w/w) of about 1:1:10:10.

In certain embodiments, the composition comprises the first mRNA, the second mRNA, the third mRNA, and the fourth mRNA present in the ratio (w/w) of about 1:1:2:2 to about 1:1:10:10.

In certain embodiments, the ratio is expressed in micrograms (μg).

In certain embodiments, the composition comprises the first mRNA, the second mRNA, the third mRNA, and the fourth mRNA present in a ratio (w/w) of about 32 micrograms of the first mRNA, to about 32 micrograms of the second mRNA, to about 64 micrograms of the third mRNA, to about 64 micrograms of the fourth mRNA.

In certain embodiments, the composition comprises the first mRNA, the second mRNA, the third mRNA, and the fourth mRNA present in a ratio (w/w) of about 16 micrograms of the first mRNA, to about 16 micrograms of the second mRNA, to about 64 micrograms of the third mRNA, to about 64 micrograms of the fourth mRNA.

In certain embodiments, the composition comprises the first mRNA, the second mRNA, the third mRNA, and the fourth mRNA present in a ratio (w/w) of about 16 micrograms of the first mRNA, to about 16 micrograms of the second mRNA, to about 96 micrograms of the third mRNA, to about 96 micrograms of the fourth mRNA.

In certain embodiments, the composition comprises 130 micrograms of the mRNA in total.

In certain embodiments, the composition comprises 160 micrograms of the mRNA in total.

In certain embodiments, the composition comprises 200 micrograms of the mRNA in total.

In certain embodiments, the composition comprises 224 micrograms of the mRNA in total.

In certain embodiments, the composition comprises 130 micrograms to 224 micrograms of the mRNA in total.

In certain embodiments, the composition comprises less than 100 micrograms of the mRNA in total.

In certain embodiments, the composition comprises less than 150 micrograms of the mRNA in total.

In certain embodiments, the composition comprises less than 200 micrograms of the mRNA in total.

In certain embodiments, the first mRNA, the second mRNA, the third mRNA, and/or the fourth mRNA are not covalently linked to one another.

In certain embodiments, the first mRNA, the second mRNA, the third mRNA, and/or the fourth mRNA are covalently linked to one another.

In certain embodiments, the first mRNA, the second mRNA, the third mRNA, and/or the fourth mRNA are formulated into an LNP.

In certain embodiments, the LNP comprises at least one cationic lipid.

In certain embodiments, the cationic lipid is biodegradable.

In certain embodiments, the cationic lipid is not biodegradable.

In certain embodiments, the cationic lipid is cleavable.

In certain embodiments, the cationic lipid is not cleavable.

In certain embodiments, the cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315) and IM-001.

In certain embodiments, the cationic lipid is cKK-E10.

In certain embodiments, the cationic lipid is GL-HEPES-E3-E12-DS-4-E10.

In certain embodiments, the cationic lipid is IM-001.

In certain embodiments, the LNP further comprises a polyethylene glycol (PEG) conjugated (PEGylated) lipid, a cholesterol-based lipid, and a helper lipid.

In certain embodiments, the LNP comprises: a cationic lipid at a molar ratio of 35% to 55%; a polyethylene glycol (PEG) conjugated (PEGylated) lipid at a molar ratio of 0.25% to 2.75%; a cholesterol-based lipid at a molar ratio of 20% to 45%; and a helper lipid at a molar ratio of 5% to 35%, wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%, wherein all of the molar ratios are relative to the total lipid content of the LNP.

In certain embodiments, the PEGylated lipid is dimyristoyl-PEG2000 (DMG-PEG2000) or 2-[(polyethylene glycol)-2000]-N,N-ditetradecylacetamide (ALC-0159).

In certain embodiments, the cholesterol-based lipid is cholesterol.

In certain embodiments, the helper lipid is 1,2-dioleoyl-SN-glycero-3-phosphoethanolamine (DOPE) or 1,2-distearoyl-sn-glycero-3-phosphocholine (DSPC).

In certain embodiments, the LNP comprises: GL-HEPES-E3-E12-DS-4-E10 at a molar ratio of 40%; DMG-PEG2000 at a molar ratio of 1.5%; cholesterol at a molar ratio of 28.5%; and DOPE at a molar ratio of 30%, wherein all of the molar ratios are relative to the total lipid content of the LNP.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%, wherein all of the molar ratios are relative to the total lipid content of the LNP.

In certain embodiments, the LNP comprises: IM-001 at a molar ratio of 40%; DMG-PEG2000 at a molar ratio of 1.5%; cholesterol at a molar ratio of 28.5%; and DOPE at a molar ratio of 30%, wherein all of the molar ratios are relative to the total lipid content of the LNP.

In certain embodiments, the LNP has an average diameter of 30 nm to 200 nm.

In certain embodiments, the LNP has an average diameter of 80 nm to 150 nm.

In certain embodiments, the first mRNA encodes an HA antigen of the influenza A subtype H1N1.

In certain embodiments, the second mRNA encodes an HA antigen of the influenza A subtype H3N2.

In certain embodiments, the third mRNA encodes an HA antigen of the influenza B Victoria-lineage strain.

In certain embodiments, the fourth mRNA encodes an HA antigen of the influenza B Yamagata-lineage strain.

In certain embodiments, at least one of the mRNAs comprises a codon-optimized ORF.

In certain embodiments, at least one of the mRNAs comprises at least one 5′ untranslated region (5′ UTR), at least one 3′ untranslated region (3′ UTR), and at least one polyadenylation (poly(A)) sequence.

In certain embodiments, at least one of the mRNAs comprises at least one chemical modification.

In certain embodiments, at least 20%, at least 30%, at least 40%, at least 50%, at least 60%, at least 70%, at least 80%, at least 85%, at least 90%, at least 95%, or 100% of the uracil nucleotides in at least one of the mRNAs are chemically modified.

In certain embodiments, at least 20%, at least 30%, at least 40%, at least 50%, at least 60%, at least 70%, at least 80%, at least 85%, at least 90%, at least 95%, or 100% of the uracil nucleotides in at least one of the ORFs are chemically modified.

In certain embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In certain embodiments, the chemical modification is selected from the group consisting of pseudouridine, N1-methylpseudouridine, 5-methylcytosine, 5-methoxyuridine, and a combination thereof.

In certain embodiments, the chemical modification is N1-methylpseudouridine.

In certain embodiments, the disclosure provides a method of administering to a subject in need thereof any of the compositions described above.

In certain embodiments, the disclosure provides a method of eliciting an immune response to influenza A or protecting a subject against an influenza A infection, comprising administering to the subject any of the compositions described above.

In certain embodiments, the disclosure provides a method of eliciting an immune response to influenza B or protecting a subject against an influenza B infection, comprising administering to the subject any of the compositions described above.

In certain embodiments, the subject has a comparable serum concentration of neutralizing antibodies against influenza A after administration of any of the compositions described above, relative to a subject that is administered a protein influenza A vaccine.

In certain embodiments, the subject has a comparable serum concentration of neutralizing antibodies against influenza B after administration of any of the compositions described above, relative to a subject that is administered a protein influenza B vaccine.

In certain embodiments, the composition increases the serum concentration of neutralizing antibodies in a subject against influenza A.

In certain embodiments, the composition increases the serum concentration of neutralizing antibodies in a subject against influenza B.

In certain embodiments, the composition increases the serum concentration of neutralizing antibodies in a subject against influenza A H1N1 and/or influenza A H3N2.

In certain embodiments, the composition increases the serum concentration of neutralizing antibodies in a subject against influenza B Yamagata-lineage and/or Victoria-lineage.

In certain embodiments, the disclosure provides a composition as disclosed herein for use in a method of eliciting an immune response to influenza A or protecting a subject against influenza A infection.

In certain embodiments, the disclosure provides a composition as disclosed herein for use in a method of eliciting an immune response to influenza B or protecting a subject against influenza B infection.

In certain embodiments, the disclosure provides for the use of any of the compositions described herein in the manufacture of a medicament for eliciting an immune response to influenza A or protecting a subject against influenza A infection.

In certain embodiments, the disclosure provides for the use of any of the compositions described herein in the manufacture of a medicament for eliciting an immune response to influenza B or protecting a subject against influenza B infection.

In another aspect, the disclosure provides for a composition comprising at least three messenger RNAs (mRNAs), wherein: (i) a first mRNA encodes a hemagglutinin (HA) antigen of a first influenza A virus; (ii) a second mRNA encodes an HA antigen of a second influenza A virus, wherein the first influenza A virus and the second influenza A virus are of different subtypes; and (iii) a third mRNA encodes an HA antigen of a first influenza B virus, wherein the mRNA encoding the HA antigen of the influenza A virus is present in a different ratio (w/w) than the mRNA encoding the HA antigen of the influenza B virus, and wherein the first mRNA, the second mRNA, and the third mRNA are formulated into a lipid nanoparticle (LNP) comprising: OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315) or IM-001 at a molar ratio of 40%; DMG-PEG2000 at a molar ratio of 1.5%; cholesterol at a molar ratio of 28.5%; and DOPE at a molar ratio of 30%.

In an embodiment, the disclosure provides a composition comprising at least three messenger RNAs (mRNAs), wherein: (i) a first mRNA encodes a hemagglutinin (HA) antigen of a first influenza A virus; (ii) a second mRNA encodes an HA antigen of a second influenza A virus, wherein the first influenza A virus and the second influenza A virus are of different subtypes; and (iii) a third mRNA encodes an HA antigen of a first influenza B virus, wherein the mRNA encoding the HA antigen of the influenza A virus is present in a different ratio (w/w) than the mRNA encoding the HA antigen of the influenza B virus, and wherein the first mRNA, the second mRNA, and the third mRNA are formulated into a lipid nanoparticle (LNP) comprising GL-HEPES-E3-E12-DS-4-E10 at a molar ratio of 40%; DMG-PEG2000 at a molar ratio of 1.5%; cholesterol at a molar ratio of 28.5%; and DOPE at a molar ratio of 30%.

In an embodiment, the disclosure provides a composition comprising at least three messenger RNAs (mRNAs), wherein: (i) a first mRNA encodes a hemagglutinin (HA) antigen of a first influenza A virus; (ii) a second mRNA encodes an HA antigen of a second influenza A virus, wherein the first influenza A virus and the second influenza A virus are of different subtypes; and (iii) a third mRNA encodes an HA antigen of a first influenza B virus, wherein the mRNA encoding the HA antigen of the influenza A virus is present in a different ratio (w/w) than the mRNA encoding the HA antigen of the influenza B virus, wherein the ratio of the mRNAs is 1:1:2 (w/w) respectively, and wherein the first mRNA, the second mRNA, and the third mRNA are formulated into a lipid nanoparticle (LNP) comprising GL-HEPES-E3-E12-DS-4-E10 at a molar ratio of 40%; DMG-PEG2000 at a molar ratio of 1.5%; cholesterol at a molar ratio of 28.5%; and DOPE at a molar ratio of 30%.

In an embodiment, the disclosure provides a composition comprising at least three messenger RNAs (mRNAs), wherein: (i) a first mRNA encodes a hemagglutinin (HA) antigen of a first influenza A virus; (ii) a second mRNA encodes an HA antigen of a second influenza A virus, wherein the first influenza A virus and the second influenza A virus are of different subtypes; and (iii) a third mRNA encodes an HA antigen of a first influenza B virus, wherein the mRNA encoding the HA antigen of the influenza A virus is present in a different ratio (w/w) than the mRNA encoding the HA antigen of the influenza B virus, and wherein the first mRNA, the second mRNA, and the third mRNA are formulated into a lipid nanoparticle (LNP) comprising IM-01 at a molar ratio of 40%; DMG-PEG2000 at a molar ratio of 1.5%; cholesterol at a molar ratio of 28.5%; and DOPE at a molar ratio of 30%.

In an embodiment, the disclosure provides a composition comprising at least three messenger RNAs (mRNAs), wherein: (i) a first mRNA encodes a hemagglutinin (HA) antigen of a first influenza A virus; (ii) a second mRNA encodes an HA antigen of a second influenza A virus, wherein the first influenza A virus and the second influenza A virus are of different subtypes; and (iii) a third mRNA encodes an HA antigen of a first influenza B virus, wherein the mRNA encoding the HA antigen of the influenza A virus is present in a different ratio (w/w) than the mRNA encoding the HA antigen of the influenza B virus, wherein the ratio of the mRNAs is 1:1:2 (w/w) respectively, and wherein the first mRNA, the second mRNA, and the third mRNA are formulated into a lipid nanoparticle (LNP) comprising IM-01 at a molar ratio of 40%; DMG-PEG2000 at a molar ratio of 1.5%; cholesterol at a molar ratio of 28.5%; and DOPE at a molar ratio of 30%.

In certain embodiments, the composition comprises a fourth mRNA encoding an HA antigen of a second influenza B virus, wherein the first influenza B virus and the second influenza B virus are of different lineages, and wherein the first mRNA, the second mRNA, the third mRNA, and the fourth mRNA are formulated into a lipid nanoparticle (LNP) comprising: OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315) or IM-001 at a molar ratio of 40%; DMG-PEG2000 at a molar ratio of 1.5%; cholesterol at a molar ratio of 28.5%; and DOPE at a molar ratio of 30%.

In certain embodiments, the composition comprises the first mRNA, the second mRNA, and the third mRNA present in the ratio (w/w) of about 1:1:2.

In certain embodiments, the composition comprises the first mRNA, the second mRNA, and the third mRNA present in the ratio (w/w) of about 1:1:3.

In certain embodiments, the composition comprises the first mRNA, the second mRNA, and the third mRNA present in the ratio (w/w) of about 1:1:4.

In certain embodiments, the composition comprises the first mRNA, the second mRNA, and the third mRNA present in the ratio (w/w) of about 1:1:5.

In certain embodiments, the composition comprises the first mRNA, the second mRNA, and the third mRNA present in the ratio (w/w) of about 1:1:6.

In certain embodiments, the composition comprises the first mRNA, the second mRNA, and the third mRNA present in the ratio (w/w) of about 1:1:7.

In certain embodiments, the composition comprises the first mRNA, the second mRNA, and the third mRNA present in the ratio (w/w) of about 1:1:8.

In certain embodiments, the composition comprises the first mRNA, the second mRNA, and the third mRNA present in the ratio (w/w) of about 1:1:9.

In certain embodiments, the composition comprises the first mRNA, the second mRNA, and the third mRNA present in the ratio (w/w) of about 1:1:10.

In certain embodiments, the composition comprises the first mRNA, the second mRNA, and the third mRNA present in the ratio (w/w) of about 1:1:2 to about 1:1:10.

In certain embodiments, the composition comprises the first mRNA, the second mRNA, the third mRNA, and the fourth mRNA present in the ratio (w/w) of about 1:1:2:2.

In certain embodiments, the composition comprises the first mRNA, the second mRNA, the third mRNA, and the fourth mRNA present in the ratio (w/w) of about 1:1:3:3.

In certain embodiments, the composition comprises the first mRNA, the second mRNA, the third mRNA, and the fourth mRNA present in the ratio (w/w) of about 1:1:4:4.

In certain embodiments, the composition comprises the first mRNA, the second mRNA, the third mRNA, and the fourth mRNA present in the ratio (w/w) of about 1:1:5:5.

In certain embodiments, the composition comprises the first mRNA, the second mRNA, the third mRNA, and the fourth mRNA present in the ratio (w/w) of about 1:1:6:6.

In certain embodiments, the composition comprises the first mRNA, the second mRNA, the third mRNA, and the fourth mRNA present in the ratio (w/w) of about 1:1:7:7.

In certain embodiments, the composition comprises the first mRNA, the second mRNA, the third mRNA, and the fourth mRNA present in the ratio (w/w) of about 1:1:8:8.

In certain embodiments, the composition comprises the first mRNA, the second mRNA, the third mRNA, and the fourth mRNA present in the ratio (w/w) of about 1:1:9:9.

In certain embodiments, the composition comprises the first mRNA, the second mRNA, the third mRNA, and the fourth mRNA present in the ratio (w/w) of about 1:1:10:10.

In certain embodiments, the composition comprises the first mRNA, the second mRNA, the third mRNA, and the fourth mRNA present in the ratio (w/w) of about 1:1:2:2 to about 1:1:10:10.

In certain embodiments, the ratio is expressed in micrograms (μg).

In certain embodiments, the composition comprises the first mRNA, the second mRNA, the third mRNA, and the fourth mRNA present in a ratio (w/w) of about 32 micrograms of the first mRNA, to about 32 micrograms of the second mRNA, to about 64 micrograms of the third mRNA, to about 64 micrograms of the fourth mRNA.

In certain embodiments, the composition comprises the first mRNA, the second mRNA, the third mRNA, and the fourth mRNA present in a ratio (w/w) of about 16 micrograms of the first mRNA, to about 16 micrograms of the second mRNA, to about 64 micrograms of the third mRNA, to about 64 micrograms of the fourth mRNA.

In certain embodiments, the composition comprises the first mRNA, the second mRNA, the third mRNA, and the fourth mRNA present in a ratio (w/w) of about 16 micrograms of the first mRNA, to about 16 micrograms of the second mRNA, to about 96 micrograms of the third mRNA, to about 96 micrograms of the fourth mRNA.

In certain embodiments, the composition comprises less than 100 micrograms of the mRNA in total.

In certain embodiments, the composition comprises less than 150 micrograms of the mRNA in total.

In certain embodiments, the composition comprises less than 200 micrograms of the mRNA in total.

In certain embodiments, the composition comprise less than 250 micrograms of the mRNA in total.

In certain embodiments, the composition comprises 100 to 250 micrograms of the mRNA in total.

In certain embodiments, the first mRNA, the second mRNA, the third mRNA, and/or the fourth mRNA are not covalently linked to one another.

In certain embodiments, the first mRNA, the second mRNA, the third mRNA, and/or the fourth mRNA are covalently linked to one another.

In certain embodiments, the LNP has an average diameter of 30 nm to 200 nm.

In certain embodiments, the LNP has an average diameter of 80 nm to 150 nm.

In certain embodiments, the first mRNA encodes an HA antigen of the influenza A subtype H1N1.

In certain embodiments, the second mRNA encodes an HA antigen of the influenza A subtype H3N2.

In certain embodiments, the third mRNA encodes an HA antigen of the influenza B Victoria-lineage strain.

In certain embodiments, the fourth mRNA encodes an HA antigen of the influenza B Yamagata-lineage strain.

In certain embodiments, at least one of the mRNAs comprises a codon-optimized ORF.

In certain embodiments, at least one of the mRNAs comprises at least one 5′ untranslated region (5′ UTR), at least one 3′ untranslated region (3′ UTR), and at least one polyadenylation (poly(A)) sequence.

In certain embodiments, at least one of the mRNAs comprises at least one chemical modification.

In certain embodiments, at least 20%, at least 30%, at least 40%, at least 50%, at least 60%, at least 70%, at least 80%, at least 85%, at least 90%, at least 95%, or 100% of the uracil nucleotides in at least one of the mRNAs are chemically modified.

In certain embodiments, at least 20%, at least 30%, at least 40%, at least 50%, at least 60%, at least 70%, at least 80%, at least 85%, at least 90%, at least 95%, or 100% of the uracil nucleotides in at least one of the ORFs are chemically modified.

In certain embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In certain embodiments, the chemical modification is selected from the group consisting of pseudouridine, N1-methylpseudouridine, 5-methylcytosine, 5-methoxyuridine, and a combination thereof.

In certain embodiments, the chemical modification is N1-methylpseudouridine.

In certain embodiments, the disclosure provides a method of administering to a subject in need thereof any of the compositions described above.

In certain embodiments, the disclosure provides a method of eliciting an immune response to influenza A or protecting a subject against an influenza A infection, comprising administering to the subject any of the compositions described above.

In certain embodiments, the disclosure provides a method of eliciting an immune response to influenza B or protecting a subject against an influenza B infection, comprising administering to the subject any of the compositions described above.

In certain embodiments, the subject has a comparable serum concentration of neutralizing antibodies against influenza A after administration of any of the compositions described above, relative to a subject that is administered a protein influenza A vaccine.

In certain embodiments, the subject has a comparable serum concentration of neutralizing antibodies against influenza B after administration of any of the compositions described above, relative to a subject that is administered a protein influenza B vaccine.

In certain embodiments, the composition increases the serum concentration of neutralizing antibodies in a subject against influenza A.

In certain embodiments, the composition increases the serum concentration of neutralizing antibodies in a subject against influenza B.

In certain embodiments, the composition increases the serum concentration of neutralizing antibodies in a subject against influenza A H1N1 and/or influenza A H3N2.

In certain embodiments, the composition increases the serum concentration of neutralizing antibodies in a subject against influenza B Yamagata-lineage and/or Victoria-lineage.

In certain embodiments, the disclosure provides composition as disclosed herein for use in a method of eliciting an immune response to influenza A or protecting a subject against influenza A infection.

In certain embodiments, the disclosure provides a composition as disclosed herein for use in a method of eliciting an immune response to influenza B or protecting a subject against influenza B infection.

In certain embodiments, the disclosure provides for the use of any of the compositions described herein in the manufacture of a medicament for eliciting an immune response to influenza A or protecting a subject against influenza A infection.

In certain embodiments, the disclosure provides for the use of any of the compositions described herein in the manufacture of a medicament for eliciting an immune response to influenza B or protecting a subject against influenza B infection.

In another aspect, the present disclosure provides a method comprising administering to a human subject a composition comprising at least three messenger RNAs (mRNAs), wherein: (i) a first mRNA encodes a hemagglutinin (HA) antigen of a first influenza A virus; (ii) a second mRNA encodes an HA antigen of a second influenza A virus, wherein the first influenza A virus and the second influenza A virus are of different subtypes; and (iii) a third mRNA encodes an HA antigen of a first influenza B virus, wherein the mRNA encoding the HA antigen of the influenza A virus is present in a different ratio (w/w) than the mRNA encoding the HA antigen of the influenza B virus, and wherein the first mRNA, the second mRNA, and the third mRNA, are formulated into a lipid nanoparticle (LNP) comprising: OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315) or IM-001 at a molar ratio of 40%; DMG-PEG2000 at a molar ratio of 1.5%; cholesterol at a molar ratio of 28.5%; and DOPE at a molar ratio of 30%.

In certain embodiments, the method comprises administering the composition comprising a fourth mRNA that encodes an HA antigen of a second influenza B virus, wherein the first influenza B virus and the second influenza B virus are of different lineages, and wherein the first mRNA, the second mRNA, the third mRNA, and the fourth mRNA are formulated into a lipid nanoparticle (LNP) comprising: OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315) or IM-001 at a molar ratio of 40%; DMG-PEG2000 at a molar ratio of 1.5%; cholesterol at a molar ratio of 28.5%; and DOPE at a molar ratio of 30%.

In certain embodiments, the composition comprises the first mRNA, the second mRNA, and the third mRNA present in the ratio (w/w) of about 1:1:2.

In certain embodiments, the composition comprises the first mRNA, the second mRNA, and the third mRNA present in the ratio (w/w) of about 1:1:3.

In certain embodiments, the composition comprises the first mRNA, the second mRNA, and the third mRNA present in the ratio (w/w) of about 1:1:4.

In certain embodiments, the composition comprises the first mRNA, the second mRNA, and the third mRNA present in the ratio (w/w) of about 1:1:5.

In certain embodiments, the composition comprises the first mRNA, the second mRNA, and the third mRNA present in the ratio (w/w) of about 1:1:6.

In certain embodiments, the composition comprises the first mRNA, the second mRNA, and the third mRNA present in the ratio (w/w) of about 1:1:7.

In certain embodiments, the composition comprises the first mRNA, the second mRNA, and the third mRNA present in the ratio (w/w) of about 1:1:8.

In certain embodiments, the composition comprises the first mRNA, the second mRNA, and the third mRNA present in the ratio (w/w) of about 1:1:9.

In certain embodiments, the composition comprises the first mRNA, the second mRNA, and the third mRNA present in the ratio (w/w) of about 1:1:10.

In certain embodiments, the composition comprises the first mRNA, the second mRNA, and the third mRNA present in the ratio (w/w) of about 1:1:2 to about 1:1:10.

In certain embodiments, the composition comprises the first mRNA, the second mRNA, the third mRNA, and the fourth mRNA present in the ratio (w/w) of about 1:1:2:2.

In certain embodiments, the composition comprises the first mRNA, the second mRNA, the third mRNA, and the fourth mRNA present in the ratio (w/w) of about 1:1:3:3.

In certain embodiments, the composition comprises the first mRNA, the second mRNA, the third mRNA, and the fourth mRNA present in the ratio (w/w) of about 1:1:4:4.

In certain embodiments, the composition comprises the first mRNA, the second mRNA, the third mRNA, and the fourth mRNA present in the ratio (w/w) of about 1:1:5:5.

In certain embodiments, the composition comprises the first mRNA, the second mRNA, the third mRNA, and the fourth mRNA present in the ratio (w/w) of about 1:1:6:6.

In certain embodiments, the composition comprises the first mRNA, the second mRNA, the third mRNA, and the fourth mRNA present in the ratio (w/w) of about 1:1:7:7.

In certain embodiments, the composition comprises the first mRNA, the second mRNA, the third mRNA, and the fourth mRNA present in the ratio (w/w) of about 1:1:8:8.

In certain embodiments, the composition comprises the first mRNA, the second mRNA, the third mRNA, and the fourth mRNA present in the ratio (w/w) of about 1:1:9:9.

In certain embodiments, the composition comprises the first mRNA, the second mRNA, the third mRNA, and the fourth mRNA present in the ratio (w/w) of about 1:1:10:10.

In certain embodiments, the composition comprises the first mRNA, the second mRNA, the third mRNA, and the fourth mRNA present in the ratio (w/w) of about 1:1:2:2 to about 1:1:10:10.

In certain embodiments, the ratio is expressed in micrograms (μg).

In certain embodiments, the composition comprises the first mRNA, the second mRNA, the third mRNA, and the fourth mRNA present in a ratio (w/w) of about 32 micrograms of the first mRNA, to about 32 micrograms of the second mRNA, to about 64 micrograms of the third mRNA, to about 64 micrograms of the fourth mRNA.

In certain embodiments, the composition comprises the first mRNA, the second mRNA, the third mRNA, and the fourth mRNA present in a ratio (w/w) of about 16 micrograms of the first mRNA, to about 16 micrograms of the second mRNA, to about 64 micrograms of the third mRNA, to about 64 micrograms of the fourth mRNA.

In certain embodiments, the composition comprises the first mRNA, the second mRNA, the third mRNA, and the fourth mRNA present in a ratio (w/w) of about 16 micrograms of the first mRNA, to about 16 micrograms of the second mRNA, to about 96 micrograms of the third mRNA, to about 96 micrograms of the fourth mRNA.

In certain embodiments, the composition comprises less than 100 micrograms of the mRNA in total.

In certain embodiments, the composition comprises less than 150 micrograms of the mRNA in total.

In certain embodiments, the composition comprises less than 200 micrograms of the mRNA in total.

In certain embodiments, the composition comprises less than 250 micrograms of the mRNA in total.

In certain embodiments, the composition comprises 100 to 250 micrograms of the mRNA in total.

In certain embodiments, the first mRNA, the second mRNA, the third mRNA, and/or the fourth mRNA are not covalently linked to one another.

In certain embodiments, the first mRNA, the second mRNA, the third mRNA, and/or the fourth mRNA are covalently linked to one another.

In certain embodiments, the LNP has an average diameter of 30 nm to 200 nm. In certain embodiments, the LNP has an average diameter of 80 nm to 150 nm.

In certain embodiments, the first mRNA encodes an HA antigen of the influenza A subtype H1N1.

In certain embodiments, the second mRNA encodes an HA antigen of the influenza A subtype H3N2.

In certain embodiments, the third mRNA encodes an HA antigen of the influenza B Victoria-lineage strain.

In certain embodiments, the fourth mRNA encodes an HA antigen of the influenza B Yamagata-lineage strain.

In certain embodiments, at least one of the mRNAs comprises a codon-optimized ORF.

In certain embodiments, at least one of the mRNAs comprises at least one 5′ untranslated region (5′ UTR), at least one 3′ untranslated region (3′ UTR), and at least one polyadenylation (poly(A)) sequence.

In certain embodiments, at least one of the mRNAs comprises at least one chemical modification.

In certain embodiments, at least 20%, at least 30%, at least 40%, at least 50%, at least 60%, at least 70%, at least 80%, at least 85%, at least 90%, at least 95%, or 100% of the uracil nucleotides in at least one of the mRNAs are chemically modified.

In certain embodiments, at least 20%, at least 30%, at least 40%, at least 50%, at least 60%, at least 70%, at least 80%, at least 85%, at least 90%, at least 95%, or 100% of the uracil nucleotides in at least one of the ORFs are chemically modified.

In certain embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In certain embodiments, the chemical modification is selected from the group consisting of pseudouridine, N1-methylpseudouridine, 5-methylcytosine, 5-methoxyuridine, and a combination thereof.

In certain embodiments, the chemical modification is N1-methylpseudouridine.

In certain embodiments, the composition is administered in an effective amount to elicit an immune response to influenza A or to protect a subject against an influenza A infection.

In certain embodiments, the composition is administered in an effective amount to elicit an immune response to influenza B or to protect a subject against an influenza B infection.

In certain embodiments, the subject has a comparable serum concentration of neutralizing antibodies against influenza A after administration of the composition, relative to a subject that is administered a protein influenza A vaccine.

In certain embodiments, the subject has a comparable serum concentration of neutralizing antibodies against influenza B after administration of the composition, relative to a subject that is administered a protein influenza B vaccine.

In certain embodiments, the composition increases the serum concentration of neutralizing antibodies in a subject against influenza A.

In certain embodiments, the composition increases the serum concentration of neutralizing antibodies in a subject against influenza B.

In certain embodiments, the composition increases the serum concentration of neutralizing antibodies in a subject against influenza A H1N1 and/or influenza A H3N2.

In certain embodiments, the composition increases the serum concentration of neutralizing antibodies in a subject against influenza B Yamagata-lineage and/or Victoria-lineage.

In another aspect, the disclosure provides a composition comprising at least three messenger RNAs (mRNAs), wherein: (i) a first mRNA encodes a hemagglutinin (HA) antigen of a first influenza A virus; (ii) a second mRNA encodes an HA antigen of a second influenza A virus, wherein the first influenza A virus and the second influenza A virus are of different subtypes; and
(iii) a third mRNA encodes an HA antigen of a first influenza B virus, wherein the first mRNA, the second mRNA, and the third mRNA are formulated into a lipid nanoparticle (LNP) comprising IM-001.

In certain embodiments, the composition comprises a fourth mRNA encoding an HA antigen of a second influenza B virus, wherein the first influenza B virus and the second influenza B virus are of different lineages, and wherein the first mRNA, the second mRNA, the third mRNA, and the fourth mRNA are formulated into a LNP comprising IM-001.

In certain embodiments, the composition comprises the first mRNA, the second mRNA, and the third mRNA present in the ratio (w/w) of about 1:1:2.

In certain embodiments, the composition comprises the first mRNA, the second mRNA, and the third mRNA present in the ratio (w/w) of about 1:1:3.

In certain embodiments, the composition comprises the first mRNA, the second mRNA, and the third mRNA present in the ratio (w/w) of about 1:1:4.

In certain embodiments, the composition comprises the first mRNA, the second mRNA, and the third mRNA present in the ratio (w/w) of about 1:1:5.

In certain embodiments, the composition comprises the first mRNA, the second mRNA, and the third mRNA present in the ratio (w/w) of about 1:1:6.

In certain embodiments, the composition comprises the first mRNA, the second mRNA, and the third mRNA present in the ratio (w/w) of about 1:1:7.

In certain embodiments, the composition comprises the first mRNA, the second mRNA, and the third mRNA present in the ratio (w/w) of about 1:1:8.

In certain embodiments, the composition comprises the first mRNA, the second mRNA, and the third mRNA present in the ratio (w/w) of about 1:1:9.

In certain embodiments, the composition comprises the first mRNA, the second mRNA, and the third mRNA present in the ratio (w/w) of about 1:1:10.

In certain embodiments, the composition comprises the first mRNA, the second mRNA, and the third mRNA present in the ratio (w/w) of about 1:1:2 to about 1:1:10.

In certain embodiments, the composition comprises the first mRNA, the second mRNA, the third mRNA, and the fourth mRNA present in the ratio (w/w) of about 1:1:2:2.

In certain embodiments, the composition comprises the first mRNA, the second mRNA, the third mRNA, and the fourth mRNA present in the ratio (w/w) of about 1:1:3:3.

In certain embodiments, the composition comprises the first mRNA, the second mRNA, the third mRNA, and the fourth mRNA present in the ratio (w/w) of about 1:1:4:4.

In certain embodiments, the composition comprises the first mRNA, the second mRNA, the third mRNA, and the fourth mRNA present in the ratio (w/w) of about 1:1:5:5.

In certain embodiments, the composition comprises the first mRNA, the second mRNA, the third mRNA, and the fourth mRNA present in the ratio (w/w) of about 1:1:6:6.

In certain embodiments, the composition comprises the first mRNA, the second mRNA, the third mRNA, and the fourth mRNA present in the ratio (w/w) of about 1:1:7:7.

In certain embodiments, the composition comprises the first mRNA, the second mRNA, the third mRNA, and the fourth mRNA present in the ratio (w/w) of about 1:1:8:8.

In certain embodiments, the composition comprises the first mRNA, the second mRNA, the third mRNA, and the fourth mRNA present in the ratio (w/w) of about 1:1:9:9.

In certain embodiments, the composition comprises the first mRNA, the second mRNA, the third mRNA, and the fourth mRNA present in the ratio (w/w) of about 1:1:10:10.

In certain embodiments, the composition comprises the first mRNA, the second mRNA, the third mRNA, and the fourth mRNA present in the ratio (w/w) of about 1:1:2:2 to about 1:1:10:10.

In certain embodiments, the ratio is expressed in micrograms (μg).

In certain embodiments, the composition comprises the first mRNA, the second mRNA, the third mRNA, and the fourth mRNA present in a ratio (w/w) of about 32 micrograms of the first mRNA, to about 32 micrograms of the second mRNA, to about 64 micrograms of the third mRNA, to about 64 micrograms of the fourth mRNA.

In certain embodiments, the composition comprises the first mRNA, the second mRNA, the third mRNA, and the fourth mRNA present in a ratio (w/w) of about 16 micrograms of the first mRNA, to about 16 micrograms of the second mRNA, to about 64 micrograms of the third mRNA, to about 64 micrograms of the fourth mRNA.

In certain embodiments, the composition comprises the first mRNA, the second mRNA, the third mRNA, and the fourth mRNA present in a ratio (w/w) of about 16 micrograms of the first mRNA, to about 16 micrograms of the second mRNA, to about 96 micrograms of the third mRNA, to about 96 micrograms of the fourth mRNA.

In certain embodiments, the composition comprises less than 100 micrograms of the mRNA in total.

In certain embodiments, the composition comprises less than 150 micrograms of the mRNA in total.

In certain embodiments, the composition comprises less than 200 micrograms of the mRNA in total.

In certain embodiments, the composition comprises less than 250 micrograms of the mRNA in total.

In certain embodiments, the composition comprises 100 to 250 micrograms of the mRNA in total.

In certain embodiments, the first mRNA, the second mRNA, the third mRNA, and/or the fourth mRNA are not covalently linked to one another.

In certain embodiments, the first mRNA, the second mRNA, the third mRNA, and/or the fourth mRNA are covalently linked to one another.

In certain embodiment, the LNP further comprises a polyethylene glycol (PEG) conjugated (PEGylated) lipid, a cholesterol-based lipid, and a helper lipid.

In certain embodiments, the LNP comprises: IM-001 at a molar ratio of 35% to 55%; a polyethylene glycol (PEG) conjugated (PEGylated) lipid at a molar ratio of 0.25% to 2.75%; a cholesterol-based lipid at a molar ratio of 20% to 45%; and a helper lipid at a molar ratio of 5% to 35%, wherein all of the molar ratios are relative to the total lipid content of the LNP.

In certain embodiments, the LNP comprises: IM-001 at a molar ratio of 40%; a PEGylated lipid at a molar ratio of 1.5%; a cholesterol-based lipid at a molar ratio of 28.5%; and a helper lipid at a molar ratio of 30%, wherein all of the molar ratios are relative to the total lipid content of the LNP.

In certain embodiments, the PEGylated lipid is dimyristoyl-PEG2000 (DMG-PEG2000) or 2-[(polyethylene glycol)-2000]-N,N-ditetradecylacetamide (ALC-0159).

In certain embodiments, the cholesterol-based lipid is cholesterol.

In certain embodiments, the helper lipid is 1,2-dioleoyl-SN-glycero-3-phosphoethanolamine (DOPE) or 1,2-distearoyl-sn-glycero-3-phosphocholine (DSPC).

In certain embodiments, the LNP has an average diameter of 30 nm to 200 nm.

In certain embodiments, the LNP has an average diameter of 80 nm to 150 nm.

In certain embodiments, the first mRNA encodes an HA antigen of the influenza A subtype H1N1.

In certain embodiments, the second mRNA encodes an HA antigen of the influenza A subtype H3N2.

In certain embodiments, the third mRNA encodes an HA antigen of the influenza B Victoria-lineage strain.

In certain embodiments, the fourth mRNA encodes an HA antigen of the influenza B Yamagata-lineage strain.

In certain embodiments, at least one of the mRNAs comprises a codon-optimized ORF.

In certain embodiments, at least one of the mRNAs comprises at least one 5′ untranslated region (5′ UTR), at least one 3′ untranslated region (3′ UTR), and at least one polyadenylation (poly(A)) sequence.

In certain embodiments, at least one of the mRNAs comprises at least one chemical modification.

In certain embodiments, at least 20%, at least 30%, at least 40%, at least 50%, at least 60%, at least 70%, at least 80%, at least 85%, at least 90%, at least 95%, or 100% of the uracil nucleotides in at least one of the mRNAs are chemically modified.

In certain embodiments, at least 20%, at least 30%, at least 40%, at least 50%, at least 60%, at least 70%, at least 80%, at least 85%, at least 90%, at least 95%, or 100% of the uracil nucleotides in at least one of the ORFs are chemically modified.

In certain embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In certain embodiments, the chemical modification is selected from the group consisting of pseudouridine, N1-methylpseudouridine, 5-methylcytosine, 5-methoxyuridine, and a combination thereof.

In certain embodiments, the chemical modification is N1-methylpseudouridine.

In certain embodiments, the disclosure provides a method of administering to a subject in need thereof any of the compositions described above.

In certain embodiments, the disclosure provides a method of eliciting an immune response to influenza A or protecting a subject against an influenza A infection, comprising administering to the subject any of the compositions described above.

In certain embodiments, the disclosure provides a method of eliciting an immune response to influenza B or protecting a subject against an influenza B infection, comprising administering to the subject any of the compositions described above.

In certain embodiments, the subject has a comparable serum concentration of neutralizing antibodies against influenza A after administration of any of the compositions described above, relative to a subject that is administered a protein influenza A vaccine.

In certain embodiments, the subject has a comparable serum concentration of neutralizing antibodies against influenza B after administration of any of the compositions described above, relative to a subject that is administered a protein influenza B vaccine.

In certain embodiments, the composition increases the serum concentration of neutralizing antibodies in a subject against influenza A.

In certain embodiments, the composition increases the serum concentration of neutralizing antibodies in a subject against influenza B.

In certain embodiments, the composition increases the serum concentration of neutralizing antibodies in a subject against influenza A H1N1 and/or influenza A H3N2.

In certain embodiments, the composition increases the serum concentration of neutralizing antibodies in a subject against influenza B Yamagata-lineage and/or Victoria-lineage.

In certain embodiments, the disclosure provides a composition as disclosed herein for use in a method of eliciting an immune response to influenza A or protecting a subject against influenza A infection.

In certain embodiments, the disclosure provides a composition as disclosed herein for use in a method of eliciting an immune response to influenza B or protecting a subject against influenza B infection.

In certain embodiments, the disclosure provides for the use of any of the compositions described herein in the manufacture of a medicament for eliciting an immune response to influenza A or protecting a subject against influenza A infection.

In certain embodiments, the disclosure provides for the use of any of the compositions described herein in the manufacture of a medicament for eliciting an immune response to influenza B or protecting a subject against influenza B infection.

### BRIEF DESCRIPTION OF THE DRAWINGS

The foregoing and other features and advantages of the present disclosure will be more fully understood from the following detailed description of illustrative embodiments taken in conjunction with the accompanying drawings.

FIG. 1 depicts interim immunogenicity data from a Phase I/II study (clinical trial NCT05624606) investigating the influenza vaccine MRT5410, a quadrivalent modified mRNA influenza vaccine encoding the hemagglutinin (HA) sequences of two influenza A strains (i.e. A/H1N1, A/H3N2) and two influenza B strains (i.e. B/Yamagata-lineage, and B/Victoria-lineage) in a 1:1:1:1 ratio and encapsulated in a lipid nanoparticle (LNP) formulation with the cationic lipid GL-HEPES-E3-E12-DS-4-E10. The vaccine was administered as a single intramuscular injection in adults 18-64 years of age and compared to the following active controls: (1) Fluzone Quadrivalent®, a quadrivalent inactivated standard dose influenza vaccine; and (2) Flublok Quadrivalent®, a quadrivalent recombinant influenza vaccine. Hemagglutinin-inhibition (HAI) geometric mean titer ratios (GMTR) and percent seroconversion were measured for the A/H1N1, A/H3N2, B/Yamagata-lineage, and B/Victoria-lineage influenza strains in each vaccine.

FIG. 2 depicts interim immunogenicity data from a Phase I/II study (clinical trial NCT05553301) investigating the influenza vaccine MRT5410, a quadrivalent modified mRNA influenza vaccine encoding the HA sequences of two influenza A strains (i.e. A/H1N1, A/H3N2) and two influenza B strains (i.e. B/Yamagata-lineage, and B/Victoria-lineage) in a 1:1:1:1 ratio and encapsulated in a LNP formulation with the cationic lipid ckk-E10. The vaccine was administered as a single intramuscular injection at two dose levels (i.e. dose level 1, dose level 2) in adults 18-64 years of age and compared to the following active controls: (1) Fluzone Quadrivalent®, a quadrivalent inactivated standard dose influenza vaccine; and (2) Flublok Quadrivalent®, a quadrivalent recombinant influenza vaccine. Hemagglutinin-inhibition (HAI) geometric mean titer ratios (GMTR) and percent seroconversion were measured for the A/H1N1, A/H3N2, B/Yamagata-lineage, and B/Victoria-lineage influenza strains in each vaccine.

### DETAILED DESCRIPTION OF THE INVENTION

The present disclosure is directed to, inter alia, novel RNA (e.g., mRNA) compositions encoding a combination of influenza A and influenza B hemagglutinin (HA) antigens, wherein the mRNA encoding the HA antigen of the influenza A virus is present in a different ratio (w/w) than the mRNA encoding the influenza B virus, and methods of eliciting an immune response by administering said compositions. In particular, the disclosures relate to mRNA encoding these antigens formulated in a lipid nanoparticle (LNP).

### I. Definitions

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, protein and nucleic acid chemistry, and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, may provide one of skill with a general dictionary of many of the terms used in this disclosure.

Units, prefixes, and symbols are denoted in their International System of Units (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

As used herein, the term “messenger RNA” or “mRNA” refers to a polynucleotide that encodes at least one polypeptide. mRNA, as used herein, encompasses both modified and unmodified RNA. mRNA may contain one or more coding and non-coding regions. A coding region is alternatively referred to as an open reading frame (ORF). Non-coding regions in mRNA include the 5′ cap, 5′ untranslated region (UTR), 3′ UTR, and a poly(A) tail. mRNA can be purified from natural sources, produced using recombinant expression systems (e.g., in vitro transcription) and optionally purified or chemically synthesized.

As used herein, the term “polypeptide” refers to any chain of amino acids, regardless of length or port-translational modification (e.g., glycosylation or phosphorylation). “Polypeptide” applies to amino acid polymers including naturally occurring amino acid polymers and non-naturally occurring amino acid polymers as well as in which one or more amino acid residues is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein and is used herein to refer to a polymer of amino acid residues.

As used herein, the term “immune response” refers to a response of a cell of the immune system, such as a B cell, T cell, dendritic cell, macrophage, or polymorphonucleocyte to a stimulus such as an antigen or vaccine. An immune response can include any cell of the body involved in a host defense response, including, for example, an epithelial cell that secretes an interferon or a cytokine. An immune response includes, but is not limited to, an innate and/or adaptive immune response.

As used herein, a “protective immune response” refers to an immune response that protects a subject from infection (e.g., prevents infection or prevents the development of disease associated with infection). Methods of measuring immune responses include measuring, for example, proliferation and/or activity of lymphocytes (such as B or T cells), secretion of cytokines or chemokines, inflammation, antibody production, and the like.

As used herein, an “antibody response” is an immune response in which antibodies are produced.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity. Antigens include the hemagglutinin (HA) antigens from both the influenza A virus, the B influenza virus, or other influenza viruses as described herein.

As used herein, an “adjuvant” refers to a substance or vehicle that enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes, killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-like receptor (TLR) agonists and costimulatory molecules.

As used herein, a “subject” refers to any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically engineered animal, and/or a clone. In certain embodiments, the subject is an adult, an adolescent, or an infant. In some embodiments, the terms “individual” or “patient” are used and are intended to be interchangeable with “subject.” In certain exemplary embodiments, the subject is a preterm newborn infant (e.g., gestational age less than 37 weeks), a newborn (e.g., 0-27 days of age), an infant or toddler (e.g., 28 days to 23 months of age), a child (e.g., 2 to 11 years of age), an adolescent (e.g., 12 to 17 years of age), an adult (e.g., 18 to 50 years of age or 18 to 64 years of age), or an elderly person (e.g., 65 years of age or older). In exemplary embodiments, the subject is 18 to 50 years of age. In other exemplary embodiments, the subject is an older adult (e.g., an adult aged 60 years of age or older).

As used herein, the term “vaccination” or “vaccinate” refers to the administration of a composition intended to generate an immune response, for example, to a disease-causing agent. Vaccination can be administered before, during, and/or after exposure to a disease-causing agent, and/or to the development of one or more symptoms, and in some embodiments, before, during, and/or shortly after exposure to the disease-causing agent. In some embodiments, vaccination includes multiple administrations, appropriately spaced in time, of a vaccinating composition.

The disclosure describes nucleic acid sequences (e.g., DNA and RNA sequences) and amino acid sequences having a certain degree of identity to a given nucleic acid sequence or amino acid sequence, respectively (a reference sequence).

### II. RNA

The vaccines of the present disclosure may comprise at least three ribonucleic acids (RNAs) each comprising an ORF encoding an influenza hemagglutinin (HA) antigen from an influenza A and/or influenza B virus. In certain embodiments, the RNAs are messenger RNAs (mRNAs) each comprising an ORF encoding an HA antigen of a first influenza A virus, an HA antigen of a second influenza A virus (wherein the first influenza A virus and the second influenza A virus are of different subtypes), and an HA antigen of an influenza B virus. In further embodiments, the RNAs are mRNAs each comprising an ORF encoding an HA antigen of a first influenza A virus, an HA antigen of a second influenza A virus (wherein the first influenza A virus and the second influenza A virus are of different subtypes), an HA antigen of a first influenza B virus, and an HA antigen of a second influenza B virus (wherein the first influenza B virus and the second influenza B virus are of different lineages). In certain embodiments, the RNAs (e.g., mRNAs) further comprises at least one 5′ UTR, 3′ UTR, poly(A) tail, and/or 5′ cap.

### A. 5′ Cap

An mRNA 5′ cap can provide resistance to nucleases found in most eukaryotic cells and promote translation efficiency. Several types of 5′ caps are known. A 7-methylguanosine cap (also referred to as “m7G” or “Cap-0”) comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G(the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

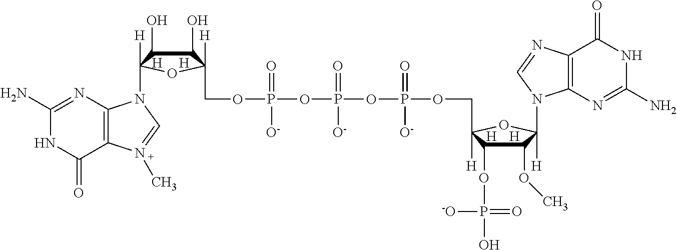

### B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 1) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of a hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′ UTR comprises a nucleic acid sequence of GGACAGAUCGCCUGGAGACGCCAUCCACGCUGUUUUGACCUCCAUAGAAGACA CCGGGACCGAUCCAGCCUCCGCGGCCGGGAACGGUGCAUUGGAACGCGGAUUC CCCGUGCCAAGAGUGACUCACCGUCCUUGACACG (SEQ ID NO: 2).

In some embodiments, the 3′ UTR comprises a nucleic acid sequence of CGGGUGGCAUCCCUGUGACCCCUCCCCAGUGCCUCUCCUGGCCCUGGAAGUUG CCACUCCAGUGCCCACCAGCCUUGUCCUAAUAAAAUUAAGUUGCAUC (SEQ ID NO: 3).

The 5′ UTR and 3′ UTR are described in further detail in International Pub. No. WO 2012/075040, incorporated herein by reference.

### C. Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence:

(SEQ ID NO: 4)
AAAAAAAAAAAAAAAAAAAAAAAAAAAAAAGCAUAUGACUAAAAAAAAAAA
AAAAAAAAAAAAAAAAAAAAAAAAAAAAAAAAAAAAAAAAAAAAAAAAAAA
AAAAAAAA.

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A) polymerases, e.g., using methods and means as described in International Pub. No. WO 2016/174271.

The nucleic acid may comprise a poly(A) tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in International Pub. No. WO 2016/091391.

In certain embodiments, the nucleic acid comprises at least one polyadenylation signal.

In various embodiments, the nucleic acid may comprise at least one poly(C) sequence.

The term “poly(C) sequence,” as used herein, is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In some embodiments, the poly(C) sequence comprises about 30 cytosine nucleotides.

### D. Chemical Modification

The mRNA disclosed herein may be modified or unmodified. In some embodiments, the mRNA may comprise at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

E. mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25 (2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

### III. Combination Influenza mRNA Vaccine Compositions

Influenza is a negative-sense, single-stranded RNA virus belonging to the Orthomyxoviridae family. Every year, influenza viruses affect millions of people globally and cause significant mortality and morbidity. Hemagglutinin (HA) is one of the main glycoproteins on the surface of influenza viruses and is integral to influenza infectivity. HA plays a major role in the attachment of influenza viruses to the host cell and fusion of viral and host membranes.

HA is composed of two subunits: HA1 and HA2. HA must be cleaved by cellular proteases to be active as a fusion protein. The cleavage of HA0 into HA1 and HA2 activates virus infectivity and is critical for influenza virus pathogenicity in humans.

Provided herein are RNA (e.g., mRNAs) compositions that target three or more influenza HA antigens.

In some embodiments, the composition comprises mRNAs encoding polypeptides derived from influenza viral proteins selected from hemagglutinin (e.g., hemagglutinin 1 (HA1) and hemagglutinin 2 (HA2), neuraminidase (NA), nucleoprotein (NP), matrix protein 1 (M1), matrix protein 2 (M2), nonstructural protein 1 (NS1), and non-structural protein 2 (NS2)). In further embodiments, the composition comprises mRNAs encoding polypeptides derived from an HA protein, from an NA protein, and from both HA and NA proteins. In other embodiments, the composition comprises mRNAs encoding antigenic polypeptides are derived from different influenza strains.

In some embodiments, the composition comprises mRNAs that encode antigens of influenza A, B and/or C viruses. In certain embodiments, the composition comprises mRNAs encoding HA and/or NA antigens of influenza A and influenza B viruses. In some embodiments, the HA antigens of influenza A viruses are selected from subtypes H1, H2, H3, H4, H5, H6, H7, H8, H9, H10, H11, H12, H13, H14, H15, H16, H17, and H18. In other embodiments, the NA antigens of influenza A viruses are selected from subtypes N1, N2, N3, N4, N5, N6, N7, N8, N9, N10, and N11. In certain embodiments, the HA and NA antigens of influenza B viruses are from the influenza B/Yamagata lineage. In some embodiments, the HA and NA antigens of influenza B viruses are from the influenza B/Victoria lineage. In some embodiments, the HA and/or NA antigens are from influenza virus strains recommended by the World Health Organization (WHO) in their annual recommendation for influenza vaccine formulations.

In certain embodiments, at least one of the one or more influenza virus proteins comprises an influenza virus HA protein having a molecular sequence identified or designed from a machine learning model, and in certain embodiments, at least one of the one or more ribonucleic acid molecules encode one or more influenza virus proteins having a molecular sequence identified or designed from a machine learning model.

In one embodiment, the composition comprises one mRNA encoding an H1 HA antigen, one mRNA encoding an H3 HA antigen, one mRNA encoding an HA antigen from the influenza B/Yamagata lineage, and one mRNA encoding an HA antigen from the influenza B/Victoria lineage.

In one embodiment, the composition comprises one mRNA encoding an H1 HA antigen, one mRNA encoding an H3 HA antigen, and one HA antigen from the influenza B/Victoria lineage.

In another embodiment, the composition further comprises one or more mRNA encoding a machine learning influenza virus HA having a molecular sequence identified or designed from a machine learning model, wherein the one or more machine learning influenza virus HA may be selected from an H1 HA, an H3 HA, an HA from a B/Victoria lineage, an HA from a B/Yamagata lineage, or a combination thereof.

When selecting one or more machine learning influenza virus HAs, any machine learning algorithm may be used. For example, envisioned herein are any of the machine learning algorithms and methods disclosed in PCT Application Nos. WO 2021/080990 A1, entitled Systems and Methods for Designing Vaccines, and WO 2021/080999 A1, entitled Systems and Methods for Predicting Biological Responses, both of which are incorporated by reference in their entireties herein.

The mRNA may be unmodified (i.e., containing only natural ribonucleotides A, U, C, and/or G linked by phosphodiester bonds), or chemically modified (e.g., including nucleotide analogs such as pseudouridines (e.g., N-1-methyl pseudouridine), 2′-fluoro ribonucleotides, and 2′-methoxy ribonucleotides, and/or phosphorothioate bonds). The mRNA molecule may comprise a 5′ cap and a polyA tail.

In some embodiments, the composition comprises at least three messenger RNAs (mRNAs), wherein the at least three mRNAs comprise an open reading frame (ORF) encoding a hemagglutinin (HA) antigen selected from the group consisting of: (i) a first mRNA encoding an HA antigen of a first influenza A virus; (ii) a second mRNA encoding an HA antigen of a second influenza A virus, wherein the first influenza A virus and the second influenza A virus are of different subtypes; and (iii) a third mRNA encoding an HA antigen of a first influenza B virus, wherein the mRNA encoding the HA antigen of the influenza A virus is present in a different ratio (w/w) than the mRNA encoding the HA antigen of the influenza B virus.

In some embodiments, the composition comprises at least three mRNAs, wherein: (i) a first mRNA encodes a HA antigen of a first influenza A virus; (ii) a second mRNA encodes an HA antigen of a second influenza A virus, wherein the first influenza A virus and the second influenza A virus are of different subtypes; and (iii) a third mRNA encodes an HA antigen of a first influenza B virus, wherein the mRNA encoding the HA antigen of the influenza A virus is present in a different ratio (w/w) than the mRNA encoding the HA antigen of the influenza B virus, and wherein the first mRNA, the second mRNA, and the third mRNA are formulated into a lipid nanoparticle (LNP) comprising: OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315) or IM-001 at a molar ratio of 40%; DMG-PEG2000 at a molar ratio of 1.5%; cholesterol at a molar ratio of 28.5%; and DOPE at a molar ratio of 30%.

In some embodiments, the composition comprises at least three mRNAs, wherein: (i) a first mRNA encodes a HA antigen of a first influenza A virus; (ii) a second mRNA encodes an HA antigen of a second influenza A virus, wherein the first influenza A virus and the second influenza A virus are of different subtypes; and (iii) a third mRNA encodes an HA antigen of a first influenza B virus, wherein the first mRNA, the second mRNA, and the third mRNA are formulated into a LNP comprising IM-001.

The compositions of any of the herein embodiments may comprise a fourth mRNA encoding an HA antigen of a second influenza B virus, wherein the first influenza B virus and the second influenza B virus are of different lineages.

The compositions in any of the herein embodiments may comprise the first mRNA, the second mRNA, the third mRNA, and/or the fourth mRNA to not be covalently linked to one another.

The compositions in any of the herein embodiments may comprise the first mRNA, the second mRNA, the third mRNA, and/or the fourth mRNA to be covalently linked to one another.

The compositions in any of the herein embodiments may comprise a first mRNA encoding an HA antigen of the influenza A subtype H1N1.

The compositions in any of the herein embodiments may comprise a second mRNA encoding an HA antigen of the influenza A subtype H3N2.

The compositions in any one of the herein embodiments may comprise a third mRNA encoding an HA antigen of the influenza B Victoria-lineage strain.

The compositions in any of the herein embodiments may comprise a fourth mRNA encoding an HA antigen of the influenza B Yamagata-lineage strain.

The compositions in any of the herein embodiments may comprise at least one mRNA comprising a codon-optimized ORF.

The compositions in any of the herein embodiments may comprise at least one mRNA comprising at least one 5′ UTR, at least one 3′ UTR, and at least one poly(A) sequence.

The compositions in any of the herein embodiments may comprise at least one mRNA comprising at least one chemical modification.

The compositions in any of the herein embodiments may comprise at least 20%, at least 30%, at least 40%, at least 50%, at least 60%, at least 70%, at least 80%, at least 85%, at least 90%, at least 95%, or 100% of the uracil nucleotides in at least one of the mRNAs to be chemically modified.

The compositions in any of the herein embodiments may comprise at least 20%, at least 30%, at least 40%, at least 50%, at least 60%, at least 70%, at least 80%, at least 85%, at least 90%, at least 95%, or 100% of the uracil nucleotides in at least one of the ORFs to be chemically modified.

The composition in any of the herein embodiments may comprise chemical modifications selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

The compositions in any of the herein embodiments may comprise chemical modifications selected from the group consisting of pseudouridine, N1-methylpseudouridine, 5-methylcytosine, 5-methoxyuridine, and a combination thereof.

The compositions in any of the herein embodiments may comprise the chemical modification N1-methylpseudouridine.

The compositions in any of the herein embodiments may comprise the first mRNA, the second mRNA, the third mRNA, and/or the fourth mRNA to be formulated into an LNP.

The compositions in any of the herein embodiments may comprise an LNP, wherein the LNP comprises at least one cationic lipid.

The compositions in any of the herein embodiments may comprise an LNP, wherein the cationic lipid is biodegradable.

The compositions in any of the herein embodiments may comprise an LNP, wherein the cationic lipid is not biodegradable.

The compositions in any of the herein embodiments may comprise an LNP, wherein the cationic lipid is cleavable.

The compositions in any of the herein embodiments may comprise an LNP, wherein the cationic lipid is not cleavable.

The compositions in any of the herein embodiments may comprise an LNP, wherein the cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315) and IM-001.

The compositions in any of the herein embodiments may comprise an LNP, wherein the cationic lipid is cKK-E10.

The compositions in any of the herein embodiments may comprise an LNP, wherein the cationic lipid is GL-HEPES-E3-E12-DS-4-E10.

The compositions in any of the herein embodiments may comprise an LNP, wherein the cationic lipid is IM-001.

The compositions in any of the herein embodiments may comprise an LNP, wherein the LNP further comprises polyethylene glycol (PEG) conjugated (PEGylated) lipid, a cholesterol-based lipid, and a helper lipid.

The compositions in any of the herein embodiments may comprise an LNP, wherein the LNP is comprised of: a cationic lipid at a molar ratio of 35% to 55%; a polyethylene glycol (PEG) conjugated (PEGylated) lipid at a molar ratio of 0.25% to 2.75%; a cholesterol-based lipid at a molar ratio of 20% to 45%; and a helper lipid at a molar ratio of 5% to 35%, wherein all of the molar ratios are relative to the total lipid content of the LNP.

The compositions in any of the herein embodiments may comprise an LNP, wherein the LNP is comprised of: a cationic lipid at a molar ratio of 40%; a PEGylated lipid at a molar ratio of 1.5%; a cholesterol-based lipid at a molar ratio of 28.5%; and a helper lipid at a molar ratio of 30%, wherein all of the molar ratios are relative to the total lipid content of the LNP.

The compositions in any of the herein embodiments may comprise an LNP, wherein the PEGylated lipid is dimyristoyl-PEG2000 (DMG-PEG2000) or 2-[(polyethylene glycol)-2000]-N,N-ditetradecylacetamide (ALC-0159).

The compositions in any of the herein embodiments may comprise an LNP, wherein the cholesterol-based lipid is cholesterol.

The compositions in any of the herein embodiments may comprise an LNP, wherein the helper lipid is 1,2-dioleoyl-SN-glycero-3-phosphoethanolamine (DOPE) or 1,2-distearoyl-sn-glycero-3-phosphocholine (DSPC).

The compositions in any of the herein embodiments may comprise an LNP, wherein the LNP is comprised of: GL-HEPES-E3-E12-DS-4-E10 at a molar ratio of 40%; DMG-PEG2000 at a molar ratio of 1.5%; cholesterol at a molar ratio of 28.5%; and DOPE at a molar ratio of 30%, wherein all of the molar ratios are relative to the total lipid content of the LNP.

The compositions in any of the herein embodiments may comprise an LNP, wherein the LNP is comprised of: cKK-E10 at a molar ratio of 40%; DMG-PEG2000 at a molar ratio of 1.5%; cholesterol at a molar ratio of 28.5%; and DOPE at a molar ratio of 30%, wherein all of the molar ratios are relative to the total lipid content of the LNP.

The compositions in any of the herein embodiments may comprise an LNP, wherein the LNP is comprised of: IM-001 at a molar ratio of 40%; DMG-PEG2000 at a molar ratio of 1.5%; cholesterol at a molar ratio of 28.5%; and DOPE at a molar ratio of 30%, wherein all of the molar ratios are relative to the total lipid content of the LNP.

The compositions in any of the herein embodiments may comprise an LNP, wherein the LNP has an average diameter of 30 nm to 200 nm.

The compositions in any of the herein embodiments may comprise an LNP, wherein the LNP has an average diameter of 80 nm to 150 nm.

Section IV of the specification further describes LNPs that can be formulated with the combination influenza mRNA vaccine compositions discussed herein and is incorporated herein.

A. Ratios of Influenza HA mRNA

As discussed in the Examples, current mRNA-based multivalent influenza vaccines under investigation encode hemagglutinin (HA) antigens from both the influenza A virus and the B influenza virus in equal ratios (w/w). While these vaccines are immunogenic, ongoing trials indicate that B strain immunogenicity is lower than A strain immunogenicity and immune responses in humans to B strain influenza viruses are sub-optimal with these vaccines. One exemplary strategy to improve the immune response against B strains is to increase the quantity/ratio of mRNA encoding the HA sequence(s) of the influenza B virus(es) compared to the mRNA encoding the HA sequences of the influenza A viruses.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of about 1:1:2.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of about 1:1:3.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of about 1:1:4.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of about 1:1:5.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of about 1:1:6.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of about 1:1:7.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of about 1:1:8.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of about 1:1:9.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of about 1:1:10.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of 1:1:2.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of 1:1:3.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of 1:1:4.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of 1:1:5.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of 1:1:6.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of 1:1:7.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of 1:1:8.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of 1:1:9.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (w/w) of 1:1:10.

The composition in any of the herein embodiments may comprise the first mRNA, the second mRNA, and the third mRNA in a ratio (w/w) of about 1:1:2.

The composition in any of the herein embodiments may comprise the first mRNA, the second mRNA, and the third mRNA in a ratio (w/w) of about 1:1:3.

The composition in any of the herein embodiments may comprise the first mRNA, the second mRNA, and the third mRNA in a ratio (w/w) of about 1:1:4.

The composition in any of the herein embodiments may comprise the first mRNA, the second mRNA, and the third mRNA in a ratio (w/w) of about 1:1:5.

The composition in any of the herein embodiments may comprise the first mRNA, the second mRNA, and the third mRNA in a ratio (w/w) of about 1:1:6.

The composition in any of the herein embodiments may comprise the first mRNA, the second mRNA, and the third mRNA in a ratio (w/w) of about 1:1:7.

The composition in any of the herein embodiments may comprise the first mRNA, the second mRNA, and the third mRNA in a ratio (w/w) of about 1:1:8.

The composition in any of the herein embodiments may comprise the first mRNA, the second mRNA, and the third mRNA in a ratio (w/w) of about 1:1:9.

The composition in any of the herein embodiments may comprise the first mRNA, the second mRNA, and the third mRNA in a ratio (w/w) of about 1:1:10.

The composition in any of the herein embodiments may comprise the first mRNA, the second mRNA, and the third mRNA in a ratio (w/w) of 1:1:2.

The composition in any of the herein embodiments may comprise the first mRNA, the second mRNA, and the third mRNA in a ratio (w/w) of 1:1:3.

The composition in any of the herein embodiments may comprise the first mRNA, the second mRNA, and the third mRNA in a ratio (w/w) of 1:1:4.

The composition in any of the herein embodiments may comprise the first mRNA, the second mRNA, and the third mRNA in a ratio (w/w) of 1:1:5.

The composition in any of the herein embodiments may comprise the first mRNA, the second mRNA, and the third mRNA in a ratio (w/w) of 1:1:6.

The composition in any of the herein embodiments may comprise the first mRNA, the second mRNA, and the third mRNA in a ratio (w/w) of 1:1:7.

The composition in any of the herein embodiments may comprise the first mRNA, the second mRNA, and the third mRNA in a ratio (w/w) of 1:1:8.

The composition in any of the herein embodiments may comprise the first mRNA, the second mRNA, and the third mRNA in a ratio (w/w) of 1:1:9.

The composition in any of the herein embodiments may comprise the first mRNA, the second mRNA, and the third mRNA in a ratio (w/w) of 1:1:10.

The composition in any of the herein embodiments may comprise the first mRNA, the second mRNA, and the third mRNA in the ratio (w/w) of about 1:1:2 to about 1:1:10.

The composition in any of the herein embodiments may comprise an mRNA ratio of the first mRNA, the second mRNA, and the third mRNA that is expressed in micrograms (μg).

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of about 1:1:2:2.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of about 1:1:3:3.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of about 1:1:4:4.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of about 1:1:5:5.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of about 1:1:6:6.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of about 1:1:7:7.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of about 1:1:8:8.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of about 1:1:9:9.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of about 1:1:10:10.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of 1:1:2:2.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of 1:1:3:3.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of 1:1:4:4.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of 1:1:5:5.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of 1:1:6:6.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of 1:1:7:7.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of 1:1:8:8.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of 1:1:9:9.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (w/w) of 1:1:10:10.

The composition in any of the herein embodiments may comprise the first mRNA, the second mRNA, the third mRNA, and the fourth mRNA in the ratio (w/w) of about 1:1:2:2.

The composition in any of the herein embodiments may comprise the first mRNA, the second mRNA, the third mRNA, and the fourth mRNA in the ratio (w/w) of about 1:1:3:3.

The composition in any of the herein embodiments may comprise the first mRNA, the second mRNA, the third mRNA, and the fourth mRNA in the ratio (w/w) of about 1:1:4:4.

The composition in any of the herein embodiments may comprise the first mRNA, the second mRNA, the third mRNA, and the fourth mRNA in the ratio (w/w) of about 1:1:5:5.

The composition in any of the herein embodiments may comprise the first mRNA, the second mRNA, the third mRNA, and the fourth mRNA in the ratio (w/w) of about 1:1:6:6.

The composition in any of the herein embodiments may comprise the first mRNA, the second mRNA, the third mRNA, and the fourth mRNA in the ratio (w/w) of about 1:1:7:7.

The composition in any of the herein embodiments may comprise the first mRNA, the second mRNA, the third mRNA, and the fourth mRNA in the ratio (w/w) of about 1:1:8:8.

The composition in any of the herein embodiments may comprise the first mRNA, the second mRNA, the third mRNA, and the fourth mRNA in the ratio (w/w) of about 1:1:9:9.

The composition in any of the herein embodiments may comprise the first mRNA, the second mRNA, the third mRNA, and the fourth mRNA in the ratio (w/w) of about 1:1:10:10.

The composition in any of the herein embodiments may comprise the first mRNA, the second mRNA, the third mRNA, and the fourth mRNA in the ratio (w/w) of 1:1:2:2.

The composition in any of the herein embodiments may comprise the first mRNA, the second mRNA, the third mRNA, and the fourth mRNA in the ratio (w/w) of 1:1:3:3.

The composition in any of the herein embodiments may comprise the first mRNA, the second mRNA, the third mRNA, and the fourth mRNA in the ratio (w/w) of 1:1:4:4.

The composition in any of the herein embodiments may comprise the first mRNA, the second mRNA, the third mRNA, and the fourth mRNA in the ratio (w/w) of 1:1:5:5.

The composition in any of the herein embodiments may comprise the first mRNA, the second mRNA, the third mRNA, and the fourth mRNA in the ratio (w/w) of 1:1:6:6.

The composition in any of the herein embodiments may comprise the first mRNA, the second mRNA, the third mRNA, and the fourth mRNA in the ratio (w/w) of 1:1:7:7.

The composition in any of the herein embodiments may comprise the first mRNA, the second mRNA, the third mRNA, and the fourth mRNA in the ratio (w/w) of 1:1:8:8.

The composition in any of the herein embodiments may comprise the first mRNA, the second mRNA, the third mRNA, and the fourth mRNA in the ratio (w/w) of 1:1:9:9.

The composition in any of the herein embodiments may comprise the first mRNA, the second mRNA, the third mRNA, and the fourth mRNA in the ratio (w/w) of 1:1:10:10.

The composition in any of the herein embodiments may comprise the first mRNA, the second mRNA, the third mRNA, and the fourth mRNA in the ratio (w/w) of about 1:1:2:2 to about 1:1:10:10.

The composition in any of the herein embodiments may comprise an mRNA ratio of the first mRNA, the second mRNA, the third mRNA, and the fourth mRNA that is expressed in micrograms (μg).

The composition in any of the herein embodiments may comprise the first mRNA, the second mRNA, the third mRNA, and the fourth mRNA in a ratio (w/w) of about 32 micrograms of the first mRNA, to about 32 micrograms of the second mRNA, to about 64 micrograms of the third mRNA, to about 64 micrograms of the fourth mRNA.

The composition in any of the herein embodiments may comprise the first mRNA, the second mRNA, the third mRNA, and the fourth mRNA in a ratio (w/w) of about 16 micrograms of the first mRNA, to about 16 micrograms of the second mRNA, to about 64 micrograms of the third mRNA, to about 64 micrograms of the fourth mRNA.

The composition in any of the herein embodiments may comprise the first mRNA, the second mRNA, the third mRNA, and the fourth mRNA in a ratio (w/w) of about 16 micrograms of the first mRNA, to about 16 micrograms of the second mRNA, to about 96 micrograms of the third mRNA, to about 96 micrograms of the fourth mRNA.

The composition in any of the herein embodiments may comprise the first mRNA, the second mRNA, the third mRNA, and the fourth mRNA in a ratio (w/w) of 32 micrograms of the first mRNA, 32 micrograms of the second mRNA, 64 micrograms of the third mRNA, and 64 micrograms of the fourth mRNA.

The composition in any of the herein embodiments may comprise the first mRNA, the second mRNA, the third mRNA, and the fourth mRNA in a ratio (w/w) of 16 micrograms of the first mRNA, 16 micrograms of the second mRNA, 64 micrograms of the third mRNA, and 64 micrograms of the fourth mRNA.

The composition in any of the herein embodiments may comprise the first mRNA, the second mRNA, the third mRNA, and the fourth mRNA in a ratio (w/w) of 16 micrograms of the first mRNA, 16 micrograms of the second mRNA, 96 micrograms of the third mRNA, and 96 micrograms of the fourth mRNA.

The composition in any of the herein embodiments may comprise 130 micrograms of the mRNA in total.

The composition in any of the herein embodiments may comprise 160 micrograms of the mRNA in total.

The composition in any of the herein embodiments may comprise 200 micrograms of the mRNA in total.

The composition in any of the herein embodiments may comprise 224 micrograms of the mRNA in total.

The composition in any of the herein embodiments may comprise 130 micrograms to 224 micrograms of the mRNA in total.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of about 1:1:2.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of about 1:1:3.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of about 1:1:4.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of about 1:1:5.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of about 1:1:6.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of about 1:1:7.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of about 1:1:8.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of about 1:1:9.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of about 1:1:10.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of 1:1:2.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of 1:1:3.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of 1:1:4.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of 1:1:5.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of 1:1:6.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of 1:1:7.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of 1:1:8.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of 1:1:9.

The composition in any of the herein embodiments may comprise an HA antigen of a first influenza A virus, an HA antigen of a second influenza A virus (in which the first influenza A virus and the second influenza A virus are of different subtypes), and an HA antigen of an influenza B virus in a mRNA ratio (e.g., μg) of 1:1:10.

The composition in any of the herein embodiments may comprise the first mRNA, the second mRNA, and the third mRNA in a ratio (e.g., μg) of about 1:1:2.

The composition in any of the herein embodiments may comprise the first mRNA, the second mRNA, and the third mRNA in a ratio (e.g., μg) of about 1:1:3.

The composition in any of the herein embodiments may comprise the first mRNA, the second mRNA, and the third mRNA in a ratio (e.g., μg) of about 1:1:4.

The composition in any of the herein embodiments may comprise the first mRNA, the second mRNA, and the third mRNA in a ratio (e.g., μg) of about 1:1:5.

The composition in any of the herein embodiments may comprise the first mRNA, the second mRNA, and the third mRNA in a ratio (e.g., μg) of about 1:1:6.

The composition in any of the herein embodiments may comprise the first mRNA, the second mRNA, and the third mRNA in a ratio (e.g., μg) of about 1:1:7.

The composition in any of the herein embodiments may comprise the first mRNA, the second mRNA, and the third mRNA in a ratio (e.g., μg) of about 1:1:8.

The composition in any of the herein embodiments may comprise the first mRNA, the second mRNA, and the third mRNA in a ratio (e.g., μg) of about 1:1:9.

The composition in any of the herein embodiments may comprise the first mRNA, the second mRNA, and the third mRNA in a ratio (e.g., μg) of about 1:1:10.

The composition in any of the herein embodiments may comprise the first mRNA, the second mRNA, and the third mRNA in a ratio (e.g., μg) of 1:1:2.

The composition in any of the herein embodiments may comprise the first mRNA, the second mRNA, and the third mRNA in a ratio (e.g., μg) of 1:1:3.

The composition in any of the herein embodiments may comprise the first mRNA, the second mRNA, and the third mRNA in a ratio (e.g., μg) of 1:1:4.

The composition in any of the herein embodiments may comprise the first mRNA, the second mRNA, and the third mRNA in a ratio (e.g., μg) of 1:1:5.

The composition in any of the herein embodiments may comprise the first mRNA, the second mRNA, and the third mRNA in a ratio (e.g., μg) of 1:1:6.

The composition in any of the herein embodiments may comprise the first mRNA, the second mRNA, and the third mRNA in a ratio (e.g., μg) of 1:1:7.

The composition in any of the herein embodiments may comprise the first mRNA, the second mRNA, and the third mRNA in a ratio (e.g., μg) of 1:1:8.

The composition in any of the herein embodiments may comprise the first mRNA, the second mRNA, and the third mRNA in a ratio (e.g., μg) of 1:1:9.

The composition in any of the herein embodiments may comprise the first mRNA, the second mRNA, and the third mRNA in a ratio (e.g., μg) of 1:1:10.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of about 1:1:2:2.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of about 1:1:3:3.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of about 1:1:4:4.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of about 1:1:5:5.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of about 1:1:6:6.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of about 1:1:7:7.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of about 1:1:8:8.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of about 1:1:9:9.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of about 1:1:10:10.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of 1:1:2:2.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of 1:1:3:3.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of 1:1:4:4.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of 1:1:5:5.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of 1:1:6:6.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of 1:1:7:7.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of 1:1:8:8.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of 1:1:9:9.

The composition in any of the herein embodiments may comprise an HA antigen of a first influenza A virus, an HA antigen of a second influenza A virus (in which the first influenza A virus and the second influenza A virus are of different subtypes), an HA antigen of a first influenza B virus, and an HA antigen of a second influenza B virus (in which the first influenza B virus and the second influenza B virus are of different lineages) in an mRNA ratio (e.g., μg) of 1:1:10:10.

The composition in any of the herein embodiments may comprise the first mRNA, the second mRNA, the third mRNA, and the fourth mRNA in the ratio (e.g., μg) of about 1:1:2:2.

The composition in any of the herein embodiments may comprise the first mRNA, the second mRNA, the third mRNA, and the fourth mRNA in the ratio (e.g., μg) of about 1:1:3:3.

The composition in any of the herein embodiments may comprise the first mRNA, the second mRNA, the third mRNA, and the fourth mRNA in the ratio (e.g., μg) of about 1:1:4:4.

The composition in any of the herein embodiments may comprise the first mRNA, the second mRNA, the third mRNA, and the fourth mRNA in the ratio (e.g., μg) of about 1:1:5:5.

The composition in any of the herein embodiments may comprise the first mRNA, the second mRNA, the third mRNA, and the fourth mRNA in the ratio (e.g., μg) of about 1:1:6:6.

The composition in any of the herein embodiments may comprise the first mRNA, the second mRNA, the third mRNA, and the fourth mRNA in the ratio (e.g., μg) of about 1:1:7:7.

The composition in any of the herein embodiments may comprise the first mRNA, the second mRNA, the third mRNA, and the fourth mRNA in the ratio (e.g., μg) of about 1:1:8:8.

The composition in any of the herein embodiments may comprise the first mRNA, the second mRNA, the third mRNA, and the fourth mRNA in the ratio (e.g., μg) of about 1:1:9:9.

The composition in any of the herein embodiments may comprise the first mRNA, the second mRNA, the third mRNA, and the fourth mRNA in the ratio (e.g., μg) of about 1:1:10:10.

The composition in any of the herein embodiments may comprise the first mRNA, the second mRNA, the third mRNA, and the fourth mRNA in the ratio (e.g., μg) of 1:1:2:2.

The composition in any of the herein embodiments may comprise the first mRNA, the second mRNA, the third mRNA, and the fourth mRNA in the ratio (e.g., μg) of 1:1:3:3.

The composition in any of the herein embodiments may comprise the first mRNA, the second mRNA, the third mRNA, and the fourth mRNA in the ratio (e.g., μg) of 1:1:4:4.

The composition in any of the herein embodiments may comprise the first mRNA, the second mRNA, the third mRNA, and the fourth mRNA in the ratio (e.g., μg) of 1:1:5:5.

The composition in any of the herein embodiments may comprise the first mRNA, the second mRNA, the third mRNA, and the fourth mRNA in the ratio (e.g., μg) of 1:1:6:6.

The composition in any of the herein embodiments may comprise the first mRNA, the second mRNA, the third mRNA, and the fourth mRNA in the ratio (e.g., μg) of 1:1:7:7.

The composition in any of the herein embodiments may comprise the first mRNA, the second mRNA, the third mRNA, and the fourth mRNA in the ratio (e.g., μg) of 1:1:8:8.

The composition in any of the herein embodiments may comprise the first mRNA, the second mRNA, the third mRNA, and the fourth mRNA in the ratio (e.g., μg) of 1:1:9:9.

The composition in any of the herein embodiments may comprise the first mRNA, the second mRNA, the third mRNA, and the fourth mRNA in the ratio (e.g., μg) of 1:1:10:10.

The composition in any of the herein embodiments may comprise the first mRNA, the second mRNA, the third mRNA, and the fourth mRNA in a ratio of about 32 micrograms of the first mRNA, to about 32 micrograms of the second mRNA, to about 64 micrograms of the third mRNA, to about 64 micrograms of the fourth mRNA.

The composition in any of the herein embodiments may comprise the first mRNA, the second mRNA, the third mRNA, and the fourth mRNA in a ratio of about 16 micrograms of the first mRNA, to about 16 micrograms of the second mRNA, to about 64 micrograms of the third mRNA, to about 64 micrograms of the fourth mRNA.

The composition in any of the herein embodiments may comprise the first mRNA, the second mRNA, the third mRNA, and the fourth mRNA in a ratio of about 16 micrograms of the first mRNA, to about 16 micrograms of the second mRNA, to about 96 micrograms of the third mRNA, to about 96 micrograms of the fourth mRNA.

The composition in any of the herein embodiments may comprise the first mRNA, the second mRNA, the third mRNA, and the fourth mRNA in a ratio of 32 micrograms of the first mRNA, 32 micrograms of the second mRNA, 64 micrograms of the third mRNA, and 64 micrograms of the fourth mRNA.

The composition in any of the herein embodiments may comprise the first mRNA, the second mRNA, the third mRNA, and the fourth mRNA in a ratio of 16 micrograms of the first mRNA, 16 micrograms of the second mRNA, 64 micrograms of the third mRNA, and 64 micrograms of the fourth mRNA.

The composition in any of the herein embodiments may comprise the first mRNA, the second mRNA, the third mRNA, and the fourth mRNA in a ratio of 16 micrograms of the first mRNA, 16 micrograms of the second mRNA, 96 micrograms of the third mRNA, and 96 micrograms of the fourth mRNA.

The composition in any of the herein embodiments may comprise 130 micrograms of the mRNA in total.

The composition in any of the herein embodiments may comprise 160 micrograms of the mRNA in total.

The composition in any of the herein embodiments may comprise 200 micrograms of the mRNA in total.

The composition in any of the herein embodiments may comprise 224 micrograms of the mRNA in total.

The composition in any of the herein embodiments may comprise 130 micrograms to 224 micrograms of the mRNA in total.

The compositions in any of the herein embodiments may comprise a ratio expressed, for example, in picograms (pg), nanograms (ng), micrograms (μg), milligrams (mg), etc.

### IV. Lipid Nanoparticle (LNP)

The LNPs of the disclosure comprise four categories of lipids: (i) an ionizable lipid (e.g., a cationic lipid); (ii) a PEGylated lipid; (iii) a cholesterol-based lipid, and (iv) a helper lipid.

### A. Cationic Lipid

An ionizable lipid facilitates mRNA encapsulation and may be a cationic lipid. A cationic lipid affords a positively charged environment at low pH to facilitate efficient encapsulation of the negatively charged mRNA drug substance. Exemplary cationic lipids are shown below in Table 1.

TABLE 1
Cationic Lipids

| Name | Structure |
|---|---|
| OF-02 (ML7) | 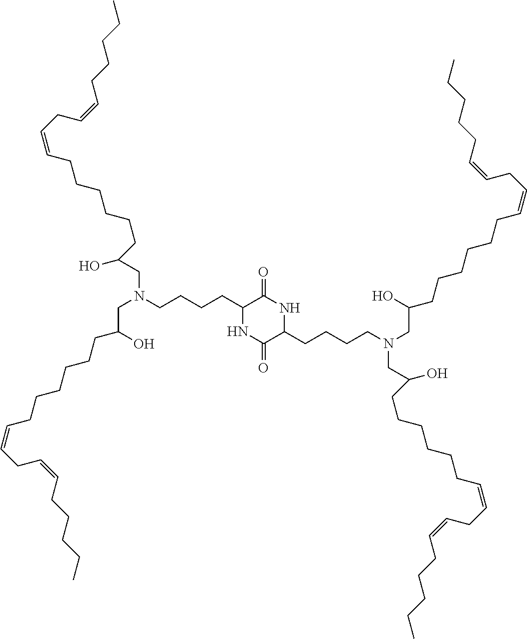 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC] |
| | OF-02 |
| cKK-E10 | 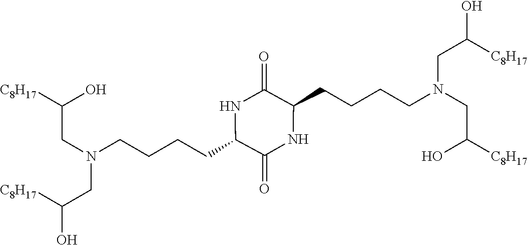 |
| GL-HEPES-E3- E10-DS-3-E18- 1 ((2-(4-(2-((3- (Bis((Z)-2- hydroxyoctadec- 9-en-1- yl)amino)propyl) disulfaneyl) ethyl)piperazin- 1-yl)ethyl 4- (bis(2- hydroxydecyl) amino)butanoate) | 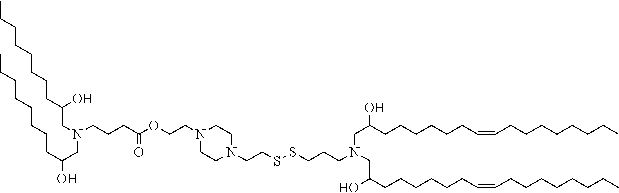 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC] |
| GL-HEPES-E3- E12-DS-4-E10 (2-(4-(2-((3- (bis(2- hydroxydecyl) amino)butyl) disulfaneyl)ethyl) piperazin-1- yl)ethyl 4- (bis(2- hydroxydodecyl) amino) butanoate) | 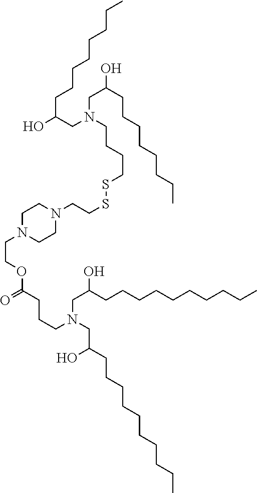 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC] |
| GL-HEPES-E3- E12-DS-3-E14 (2-(4-(2-((3- (Bis(2- hydroxytetradec yl)amino)propyl) disulfaneyl) ethyl)piperazin- 1-yl)ethyl 4- (bis(2- hydroxydodecyl) amino) butanoate) | 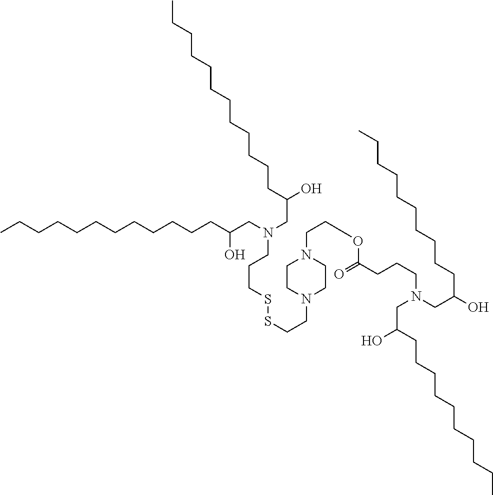 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC] |
| MC3 | 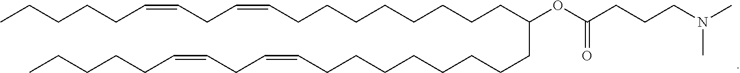 [SMILES: CCCCC/C=C\C/C=C\CCCCCCCCC(CCCCCCCC/C=C\C/C=C\CCCCC)OC(=O)CCCN(C)C] |
| SM-102 (9-heptadecanyl 8-{(2- hydroxyethyl)[6- oxo-6- (undecyloxy) hexyl]amino} octanoate) | 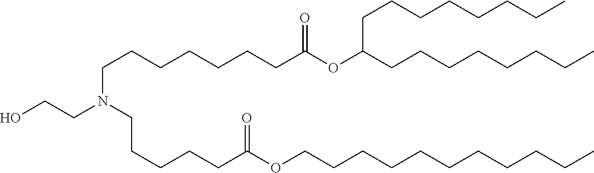 [SMILES: CCCCCCCCCCCOC(=O)CCCCCN(CCO)CCCCCCCC(=O)OC(CCCCCCCC)CCCCCCCC] |
| ALC-0315 [(4- hydroxybutyl) azanediyl]di (hexane-6,1-diyl) bis(2- hexyldecanoate) | 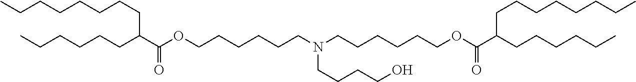 [SMILES: CCCCCCCCC(CCCCCC)C(=O)OCCCCCCN(CCCCO)CCCCCCOC(=O)C(CCCCCC)CCCCCCCC] |
| IM-001 | 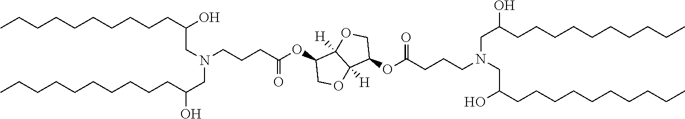 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC] |

The cationic lipid may be selected from the group comprising [ckkE10]/[OF-02], [(6Z,9Z,28Z,31Z)-heptatriaconta-6,9,28,31-tetraen-19-yl] 4-(dimethylamino)butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl] (Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl] N-[2-(dimethylamino)ethyl]carbamate (DC-Chol); tetrakis(8-methylnonyl) 3,3′,3″,3″-(((methylazanediyl) bis(propane-3,1 diyl))bis(azanetriyl))tetrapropionate (306Oi10); decyl(2-(dioctylammonio)ethyl) phosphate (9A1P9); ethyl 5,5-di((Z)-heptadec-8-en-1-yl)-1-(3-(pyrrolidin-1-yl) propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-O16B); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200); 3,6-bis(4-(bis(2-hydroxydodecyl)amino)butyl) piperazine-2,5-dione (cKK-E12); hexa (octan-3-yl) 9,9′,9″,9′″,9″″,9′″″-((((benzene-1,3,5-tricarbonyl)tris(azanediyl)) tris (propane-3,1-diyl)) tris(azanetriyl))hexanonanoate (FTT5); (((3,6-dioxopiperazine-2,5-diyl) bis(butane-4,1-diyl))bis(azanetriyl))tetrakis(ethane-2,1-diyl) (9Z,9′Z,9″Z,9′″Z,12Z,12′Z, 12″Z,12′″Z)-tetrakis (octadeca-9,12-dienoate) (OF-Deg-Lin); TT3; N1,N3,N5-tris(3-(didodecylamino) propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino) octanoate (Lipid 5); IM-001; and combinations thereof.

In certain embodiments, the cationic lipid is biodegradable.

In various embodiments, the cationic lipid is not biodegradable.

In some embodiments, the cationic lipid is cleavable.

In certain embodiments, the cationic lipid is not cleavable.

Cationic lipids are described in further detail in Dong et al. (PNAS. 111 (11): 3955-60. 2014); Fenton et al. (Adv. Mater. 28:2939. 2016); U.S. Pat. Nos. 9,512,073; and 10,201,618, each of which is incorporated herein by reference.

### B. PEGylated Lipid

The PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al. FEBS Letters 268 (1): 235-7. 1990). These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C6-C20 (e.g., C8, C10, C12, C14, C16, or C¬18) length, such as a derivatized ceramide (e.g., N-octanoyl-sphingosine-1-[succinyl(methoxypolyethylene glycol)] (C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

### C. Cholesterol-Based Lipid

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), 1,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO2011/068810), sitosterol (22,23-dihydrostigmasterol), β-sitosterol, sitostanol, fucosterol, stigmasterol (stigmasta-5,22-dien-3-ol), ergosterol; desmosterol (3ß-hydroxy-5,24-cholestadiene); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-3ß-ol); lathosterol (5α-cholest-7-en-3ß-ol); diosgenin ((3β,25R)-spirost-5-en-3-ol); campesterol (campest-5-en-3ß-ol); campestanol (5a-campestan-3b-ol); 24-methylene cholesterol (5,24 (28)-cholestadien-24-methylen-3ß-ol); cholesteryl margarate (cholest-5-en-3ß-yl heptadecanoate); cholesteryl oleate; cholesteryl stearate and other modified forms of cholesterol. In some embodiments, the cholesterol-based lipid used in the LNPs is cholesterol.

### D. Helper Lipid

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, 1-stearoyl-2-oleoyl-phosphatidyethanolamine (SOPE), or a combination thereof. In certain embodiments, the helper lipid is DOPE. In certain embodiments, the helper lipid is DSPC.

In various embodiments, the present LNPs comprise (i) a cationic lipid selected from OF-02, cKK-E10, GL-HEPES-E3-E10-DS-3-E18-1, GL-HEPES-E3-E12-DS-4-E10, or GL-HEPES-E3-E12-DS-3-E14; (ii) DMG-PEG2000; (iii) cholesterol; and (iv) DOPE.

### E. Molar Ratios of the Lipid Components

The molar ratios of the herein components are important for the LNPs' effectiveness in delivering mRNA. The molar ratio of the cationic lipid, the PEGylated lipid, the cholesterol-based lipid, and the helper lipid is A: B: C: D, where A+B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12%, such as 10%). In some embodiments, the (PEGylated lipid+cholesterol) components have the same molar amount as the helper lipid. In some embodiments, the LNPs contain a molar ratio of the cationic lipid to the helper lipid that is more than 1.

In certain embodiments, the LNP of the disclosure comprises:
a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%);
a polyethylene glycol (PEG) conjugated (PEGylated) lipid at a molar ratio of 0.25% to 2.75% or 1.00% to 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%);
a cholesterol-based lipid at a molar ratio of 20% to 45%, 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, or 50%); and
a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%),
wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the PEGylated lipid is dimyristoyl-PEG2000 (DMG-PEG2000).

In various embodiments, the cholesterol-based lipid is cholesterol.

In some embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-3-E14 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%. This LNP formulation is designated “Lipid A” herein.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid B” herein.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%. This LNP formulation is designated “Lipid C” herein.

In certain embodiments, the LNP comprises: GL-HEPES-E3-E12-DS-4-E10 (at a molar ratio of 40%; DMG-PEG2000 at a molar ratio of 1.5%; cholesterol at a molar ratio of 28.5%; and DOPE at a molar ratio of 30%. This LNP formulation is designated “Lipid D” herein.

In certain embodiments, the LNP comprises: GL-HEPES-E3-E12-DS-3-E14 at a molar ratio of 40%; DMG-PEG2000 at a molar ratio of 1.5%; cholesterol at a molar ratio of 28.5%; and DOPE at a molar ratio of 30%. This LNP formulation is designated “Lipid E” herein.

In certain embodiments, the LNP comprises: 9-heptadecanyl 8-{(2-hydroxyethyl) [6-oxo-6-(undecyloxy) hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl)azanediyl]di(hexane-6,1-diyl) bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl)azanediyl]di(hexane-6,1-diyl) bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

In certain embodiments, the LNP comprises: IM-001 at a molar ratio of 40%; DMG-PEG2000 at a molar ratio of 1.5%; cholesterol at a molar ratio of 28.5%; and DOPE at a molar ratio of 30%.

To calculate the actual amount of each lipid to be put into an LNP formulation, the molar amount of the cationic lipid is first determined based on a desired N/P ratio, where Nis the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

### F. Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients are parabens, thimerosal, thiomersal, chlorobutanol, benzalkonium chloride, and chelators (e.g., EDTA).

The LNP compositions of the present disclosure can be provided as a frozen liquid form or a lyophilized form. A variety of cryoprotectants may be used, including, without limitations, sucrose, trehalose, glucose, mannitol, mannose, dextrose, and the like. The cryoprotectant may constitute 5-30% (w/v) of the LNP composition. In some embodiments, the LNP composition comprises trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution—thawed if previously frozen, or if previously lyophilized, reconstituted in an aqueous buffered solution at bedside. The buffered solution preferably is isotonic and suitable for e.g., intramuscular or intradermal injection. In some embodiments, the buffered solution is a phosphate-buffered saline (PBS).

### V. Processes for Making LNP Vaccines

The present LNPs can be prepared by various techniques presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in Patent Application Pub. Nos. US 2011/0244026, US 2016/0038432, US 2018/0153822, US 2018/0125989, and US 2021/0046192 and can be used for making LNP vaccines. One exemplary process entails encapsulating mRNA by mixing it with a mixture of lipids, without first pre-forming the lipids into lipid nanoparticles, as described in Patent Application Pub. No. US 2016/0038432. Another exemplary process entails encapsulating mRNA by mixing pre-formed LNPs with mRNA, as described in Patent Application Pub. No. US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA and the mixed solution comprising the LNP-encapsulated mRNA. In some embodiments, the process includes the step of heating one or both of the mRNA solution and the pre-formed LNP solution, prior to the mixing step. In some embodiments, the process includes heating one or more of the solutions comprising the pre-formed LNPs, the solution comprising the mRNA and the solution comprising the LNP-encapsulated mRNA, during the mixing step. In some embodiments, the process includes the step of heating the LNP-encapsulated mRNA, after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods known in the art are available for sizing of a population of lipid nanoparticles. Preferred methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80 or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In certain embodiments, the LNP has an average diameter of 30-200 nm.

In various embodiments, the LNP has an average diameter of 80-150 nm.

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, 99% of the LNPs in the present composition have a size ranging from about 40-90 nm (e.g., about 45-85 nm, about 50-80 nm, about 55-75 nm, about 60-70 nm) or about 50-70 nm (e.g., 55-65 nm) are particular suitable for pulmonary delivery via nebulization.

In some embodiments, the dispersity, or measure of heterogeneity in size of molecules (PDI), of LNPs in a pharmaceutical composition provided by the present invention is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of 50% to 99%; or greater than about 60%, 65%, 70%, 75%, 80%, 85%, 90%, 92%, 95%, 98%, or 99%. Typically, lipid nanoparticles for use herein have an encapsulation efficiency of at least 90% (e.g., at least 91%, 92%, 93%, 94%, or 95%).

In some embodiments, an LNP has a N/P ratio of between 1 and 10. In some embodiments, a lipid nanoparticle has a N/P ratio above 1, about 1, about 2, about 3, about 4, about 5, about 6, about 7, or about 8. In further embodiments, a typical LNP herein has an N/P ratio of 4.

In some embodiments, a pharmaceutical composition according to the present invention contains at least about 0.5 μg, 1 μg, 5 μg, 10 μg, 100 μg, 500 μg, or 1000 μg of encapsulated mRNA. In some embodiments, a pharmaceutical composition contains about 0.1 μg to 1000 μg, at least about 0.5 μg, at least about 0.8 μg, at least about 1 μg, at least about 5 μg, at least about 8 μg, at least about 10 μg, at least about 50 μg, at least about 100 μg, at least about 500 μg, or at least about 1000 μg of encapsulated mRNA.

In some embodiments, mRNA can be made by chemical synthesis or by in vitro transcription (IVT) of a DNA template. An exemplary process for making and purifying mRNA is described in Example 1. In this process, in an IVT process, a cDNA template is used to produce an mRNA transcript and the DNA template is degraded by a DNase. The transcript is purified by depth filtration and tangential flow filtration (TFF). The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In particular embodiments, the aqueous buffer has 1 mM citrate, 150 mM NaCl, pH 4.5.

An exemplary, nonlimiting process for making an mRNA-LNP composition involves mixing a buffered mRNA solution with a solution of lipids in ethanol in a controlled homogeneous manner, where the ratio of lipids:mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

### VI. Packaging and Use of the mRNA-LNP Vaccines

The mRNA-LNP vaccines can be formulated or packaged for parenteral (e.g., intramuscular, intradermal, or subcutaneous) administration or nasopharyngeal (e.g., intranasal) administration. In various embodiments, the mRNA-LNP vaccines may be formulated or packaged for pulmonary administration. In various embodiments, the mRNA-LNP vaccines may be formulated or packaged for intravenous administration. The vaccine compositions may be in the form of an extemporaneous formulation, where the LNP composition is lyophilized and reconstituted with a physiological buffer (e.g., PBS) just before use. The vaccine compositions also may be shipped and provided in the form of an aqueous solution or a frozen aqueous solution and can be directly administered to subjects without reconstitution (after thawing, if previously frozen).

Accordingly, the present disclosure provides an article of manufacture, such as a kit, that provides the mRNA-LNP vaccine in a single container or provides the mRNA-LNP vaccine in one container (e.g., a first container) and a physiological buffer for reconstitution in another container (e.g., a second container). The container(s) may contain a single-use dosage or multi-use dosage. The container(s) may be pre-treated glass vials or ampules. The article of manufacture may include instructions for use as well.

In certain embodiments, the mRNA-LNP vaccine is provided for use in intramuscular (IM) injection. The vaccine can be injected into a subject at, e.g., his/her deltoid muscle in the upper arm. In some embodiments, the vaccine is provided in a pre-filled syringe or injector (e.g., single-chambered or multi-chambered). In some embodiments, the vaccine is provided for use in inhalation and is provided in a pre-filled pump, aerosolizer, or inhaler.

The mRNA-LNP vaccines can be administered to subjects in need thereof in a prophylactically effective amount, i.e., an amount that provides sufficient immune protection against a target pathogen for a sufficient amount of time (e.g., one year, two years, five years, ten years, or a lifetime). Sufficient immune protection may be, for example, prevention or alleviation of symptoms associated with infections by the pathogen. In some embodiments, multiple doses (e.g., two doses) of the vaccine are administered (e.g., injected) to subjects in need thereof to achieve the desired prophylactic effects. The doses (e.g., prime and booster doses) may be separated by an interval of at least, e.g., 2 weeks, 3 weeks, 4 weeks, one month, two months, three months, four months, five months, six months, one year, two years, five years, or ten years.

### VII. Vectors

In one aspect, disclosed herein are vectors comprising the mRNA compositions disclosed herein. The RNA sequences encoding a protein of interest (e.g., mRNA encoding an influenza HA protein) can be cloned into a number of types of vectors. For example, the nucleic acids can be cloned into a vector including, but not limited to, a plasmid, a phagemid, a phage derivative, an animal virus, and a cosmid. Vectors of particular interest can include expression vectors, replication vectors, probe generation vectors, sequencing vectors, and vectors optimized for in vitro transcription.

In certain embodiments, the vector can be used to express mRNA in a host cell. In various embodiments, the vector can be used as a template for IVT. The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14, 265-281.

In some embodiments, the vectors disclosed herein can comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein may comprise a polynucleotide sequence encoding a poly(A) sequence and/or a polyadenylation signal.

A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known.

Also disclosed herein are host cells (e.g., mammalian cells, e.g., human cells) comprising the vectors or RNA compositions disclosed herein.

Polynucleotides can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12 (8): 861-70, or the TransIT-RNA transfection Kit (Mirus, Madison, WI).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell a variety of assays may be performed.

### VIII. Self-Replicating RNA and Trans-Replicating RNA

### Self-Replicating RNA:

In one aspect, disclosed herein are self-replicating RNAs encoding an influenza protein (e.g. an influenza HA protein).

Self-replicating RNA can be produced by using replication elements derived from, e.g., alphaviruses, and substituting the structural viral proteins with a nucleotide sequence encoding a protein of interest (e.g., an influenza HA protein). A self-replicating RNA is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen (i.e., an influenza HA protein antigen), or may be transcribed to provide further transcripts with the same sense as the delivered RNA which are translated to provide in situ expression of the antigen. The overall result of this sequence of transcriptions is a large amplification in the number of the introduced replicon RNAs and so the encoded antigen becomes a major polypeptide product of the cells.

One suitable system for achieving self-replication in this manner is to use an alphavirus-based replicon. These replicons are positive stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the positive-strand delivered RNA. These negative (−)-stranded transcripts can themselves be transcribed to give further copies of the positive-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, see the following reference: International Pub. No. WO2005/113782, incorporated herein by reference.

In one embodiment, each self-replicating RNA described herein encodes (i) an RNA-dependent RNA polymerase which can transcribe RNA from the self-replicating RNA molecule and (ii) an influenza HA protein antigen. The polymerase can be an alphavirus replicase, e.g., comprising one or more of alphavirus proteins nsP1, nsP2, nsP3, and nsP4. Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins. Self-replicating RNA are described in further detail in International Pub. No. WO2011005799, incorporated herein by reference.

### Trans-Replicating RNA:

In one aspect, disclosed herein are trans-replicating RNAs encoding an influenza protein.

Trans-replicating RNA possess similar elements as the self-replicating RNA described above. However, with trans replicating RNA, two separate RNA molecules are used. A first RNA molecule encodes for the RNA replicase described above (e.g., the alphavirus replicase) and a second RNA molecule encodes for the protein of interest (e.g., an influenza protein antigen). The RNA replicase may replicate one or both of the first and second RNA molecule, thereby greatly increasing the copy number of RNA molecules encoding the protein of interest. Trans replicating RNA are described in further detail in International Pub. No. WO2017162265, incorporated herein by reference.

### IX. Pharmaceutical Compositions

RNA purified according to this disclosure can be useful as a component in pharmaceutical compositions, for example, for use as a vaccine. These compositions will typically include RNA and a pharmaceutically acceptable carrier. A pharmaceutical composition of the present disclosure can also include one or more additional components such as small molecule immunopotentiators (e.g., TLR agonists). A pharmaceutical composition of the present disclosure can also include a delivery system for the RNA, such as a liposome, an oil-in-water emulsion, or a microparticle. In some embodiments, the pharmaceutical composition comprises a lipid nanoparticle (LNP). In certain embodiments, the composition comprises an antigen-encoding nucleic acid molecule encapsulated within an LNP.

### X. Methods of Vaccination

The influenza mRNA vaccine compositions disclosed herein may be administered to a subject to induce an immune response directed against one or more influenza proteins, wherein an anti-antigen antibody titer in the subject is increased following vaccination relative to an anti-antigen antibody titer in a subject that is not vaccinated with the influenza mRNA vaccine composition disclosed herein, or relative to an alternative vaccine against influenza. An “anti-antigen antibody” is a serum antibody that binds specifically to the antigen.

In one aspect, the disclosure provides a method of eliciting an immune response to influenza or protecting a subject against influenza infection comprising administering an influenza mRNA vaccine composition described herein to a subject. In another aspect, the disclosure provides compositions for use in eliciting an immune response to influenza or in protecting a subject against influenza infection by administering the influenza mRNA vaccine compositions described herein to a subject. The disclosure also provides influenza mRNA vaccine compositions described herein for use in the manufacture of a vaccine for eliciting an immune response to influenza or for protecting a subject against influenza infection.

In one aspect, the disclosure provides a method of eliciting an immune response to influenza A or protecting a subject against an influenza A infection by administering an influenza mRNA vaccine composition described herein to a subject. In another aspect, the disclosure provides a method of eliciting an immune response to influenza B or protecting a subject against an influenza B infection by administering an influenza mRNA vaccine composition described herein to a subject.

In one aspect, the disclosure provides compositions as disclosed herein for use in a method of eliciting an immune response to influenza A or in protecting a subject against influenza A infection. In another aspect, the disclosure provides composition as disclosed herein for use in a method of eliciting an immune response to influenza B or in protecting a subject against influenza B infection.

The disclosure also provides influenza mRNA vaccine compositions described herein for use in the manufacture of a medicament for eliciting an immune response to influenza A or for protecting a subject against influenza A infection. The disclosure further provides influenza mRNA vaccine compositions described herein for use in the manufacture of a medicament for eliciting an immune response to influenza B or for protecting a subject against influenza B infection.

In another aspect, the present invention provides methods of immunizing a subject against one or more influenza viruses in a subject. The present invention further provides methods of eliciting an immune response against one or more influenza viruses in a subject. In some embodiments, the present methods comprise administering to the subject an effective amount of an influenza mRNA vaccine composition described herein to a subject.

In certain embodiments, the subject has the same or higher serum concentration of neutralizing antibodies against influenza A after administration of the influenza mRNA vaccine compositions described herein, relative to a subject that is administered a single antigenic composition comprising an mRNA encoding an influenza protein. In certain embodiments, the subject has the same or higher serum concentration of neutralizing antibodies against influenza B after administration of the influenza mRNA vaccine compositions described herein, relative to a subject that is administered a single antigenic composition comprising an mRNA encoding an influenza protein.

In certain embodiments, the subject has a comparable serum concentration of neutralizing antibodies against influenza A after administration of the influenza mRNA vaccine compositions described herein, relative to a subject that is administered a protein influenza A vaccine. In certain embodiments, the subject has a comparable serum concentration of neutralizing antibodies against influenza B after administration of the influenza mRNA vaccine compositions described herein, relative to a subject that is administered a protein influenza B vaccine.

In certain embodiments, the influenza mRNA vaccine compositions described herein increase the serum concentration of neutralizing antibodies in a subject against influenza A. In certain embodiments, the influenza mRNA vaccine compositions described herein increase the serum concentration of neutralizing antibodies in a subject against influenza B.

In certain embodiments, the influenza mRNA vaccine compositions described herein increase the serum concentration of neutralizing antibodies in a subject against influenza A H1N1 and/or influenza A H3N2. In certain embodiments, the influenza mRNA vaccine compositions described herein increase the serum concentration of neutralizing antibodies in a subject against influenza B Yamagata-lineage and/or Victoria-lineage.

In certain embodiments, the compositions described herein increase the serum concentration of neutralizing antibodies in a subject with pre-existing influenza immunity.

In various embodiments, the methods of immunizing provided herein elicit a broadly protective immune response against multiple epitopes within one or more influenza viruses. In various embodiments, the methods of immunizing provided herein elicit a broadly neutralizing immune response against one or more influenza viruses. In some embodiments, the immune response comprises an antibody response. Accordingly, in various embodiments, the influenza mRNA vaccine compositions described herein can offer broad cross-protection against different types of influenza viruses. In some embodiments, the influenza mRNA vaccine compositions described herein offer cross-protection against avian, swine, seasonal, and/or pandemic influenza viruses. In some embodiments, the influenza mRNA vaccine compositions described herein offer cross-protection against one or more influenza A, B, or C subtypes. In some embodiments, the influenza mRNA vaccine compositions described herein offer cross-protection against multiple strains of influenza A H1-subtype viruses (e.g., H1N1), influenza A H3-subtype viruses (e.g., H3N2), influenza A H5-subtype viruses (e.g., H5N1), and/or influenza B viruses (e.g., Yamagata lineage, Victoria lineage).

In some embodiments, the methods of the invention are capable of eliciting an improved immune response against one or more seasonal influenza strains. Exemplary seasonal strains include, without limitation, A/Puerto Rico/8/1934, A/Fort Monmouth/1/1947, A/Chile/1/1983, A/Texas/36/1991, A/Singapore/6/1986, A/Beijing/32/1992, A/New Caledonia/20/1999, A/Solomon Islands/03/2006, A/Brisbane/59/2007, A (H3N2) virus antigenically like the cell-propagated prototype virus A/Victoria/361/2011, A/Beijing/262/95 (H1N1)-like virus, A/Brisbane/02/2018 (H1N1)pdm09-like virus, A/Brisbane/10/2007 (H3N2)-like virus, A/California/7/2004 (H3N2)-like virus, A/California/7/2009 (H1N1)-like virus, A/California/7/2009 (H1N1)pdm09-like virus, A/Cambodia/e0826360/2020 (H3N2)-like virus, A/Darwin/6/2021 (H3N2)-like virus, A/Fujian/411/2002 (H3N2)-like virus, A/Fujian/411/2002 (H3N2)-like virus, A/Guangdong-Maonan/SWL1536/2019 (H1N1)pdm09-like virus-like virus, A/Hawaii/70/2019 (H1N1)pdm09-like virus-like virus, A/Hong Kong/2671/2019 (H3N2)-like virus, A/Hong Kong/45/2019 (H3N2)-like virus, A/Hong Kong/4801/2014 (H3N2)-like virus, A/Kansas/14/2017 (H3N2)-like virus, A/Michigan/45/2015 (H1N1)pdm09-like virus, A/Moscow/10/99 (H3N2)-like virus, A/New Caledonia/20/99 (H1N1)-like virus, A/Perth/16/2009 (H3N2)-like virus, A/Singapore/INFIMH-16-0019/2016 (H3N2)-like virus, A/Solomon Islands/3/2006 (H1N1)-like virus, A/South Australia/34/2019 (H3N2)-like virus, A/Switzerland/8060/2017 (H3N2)-like virus, A/Switzerland/9715293/2013 (H3N2)-like virus, A/Sydney/5/97 (H3N2)-like virus, A/Texas/50/2012 (H3N2)-like virus, A/Victoria/2570/2019 (H1N1)pdm09-like virus, A/Victoria/2570/2019 (H1N1)pdm09-like virus-like virus, A/Victoria/361/2011 (H3N2)-like virus, A/Wellington/1/2004 (H3N2)-like virus, A/Wisconsin/588/2019 (H1N1)pdm09-like virus, A/Wisconsin/588/2019 (H1N1)pdm09-like virus-like virus, A/Wisconsin/67/2005 (H3N2)-like virus, B/Austria/1359417/20121 (B/Victoria lineage)-like virus, B/Beijing/184/93-like virus, B/Brisbane/60/2008-like virus, B/Colorado/06/2017-like virus (B/Victoria/2/87 lineage), B/Florida/4/2006-like virus, B/Hong Kong/330/2001-like virus, B/Malaysia/2506/2004-like virus, B/Massachusetts/2/2012-like virus, B/Phuket/3073/2013 (B/Yamagata lineage)-like virus, B/Phuket/3073/2013-like virus, B/Phuket/3073/2013-like virus (B/Yamagata/16/88 lineage), B/Shangdong/7/97-like virus, B/Shanghai/361/2002-like virus, B/Sichuan/379/99-like virus, B/Washington/02/2019 (B/Victoria lineage)-like virus, B/Washington/02/2019-like (B/Victoria lineage) virus, A/Wisconsin/588/2019 (H1N1) pdm09-like virus, and B/Wisconsin/1/2010-like virus. In some embodiments, the methods of the invention are capable of eliciting an improved immune response against one or more pandemic influenza strains. Exemplary pandemic strains include, without limitation, A/California/07/2009, A/California/04/2009, A/Belgium/145/2009, A/South Carolina/01/1918, and A/New Jersey/1976. Pandemic subtypes include, in particular the H1N1, H5N1, H2N2, H3N2, H9N2, H7N7, H7N3, H7N9 and H10N7 subtypes. In some embodiments, the methods of the invention are capable of eliciting an improved immune response against one or more swine influenza strains. Exemplary swine strains include, without limitation, A/New Jersey/1976 isolates and A/California/07/2009 In some embodiments, the methods of the invention are capable of eliciting an improved immune response against one or more avian influenza strains. Exemplary avian strains include, without limitation, H5N1, H7N3, H7N7, H7N9, and H9N2. Additional influenza pandemic, seasonal, avian and/or swine strains are known in the art.

In some embodiments, the present invention provides methods of preventing or treating influenza infections by administering the influenza mRNA vaccine compositions described herein to a subject in need thereof. In some embodiments, the subject is suffering from or susceptible to an influenza infection. In some embodiments, a subject is considered to be suffering from an influenza infection if the subject is displaying one or more symptoms commonly associated with influenza infection. In some embodiments, the subject is known or believed to have been exposed to the influenza virus. In some embodiments, a subject is considered to be susceptible to an influenza infection if the subject is known or believed to have been exposed to the influenza virus. In some embodiments, a subject is known or believed to have been exposed to the influenza virus if the subject has been in contact with other individuals known or suspected to have been infected with the influenza virus and/or if the subject is or has been present in a location in which influenza infection is known or thought to be prevalent.

In various embodiments, the influenza mRNA vaccine compositions described herein may be administered prior to or after development of one or more symptoms of influenza infection. In some embodiments, the influenza mRNA vaccine compositions described herein are administered as a prophylactic. In such embodiments, the methods of the invention are effective in preventing or protecting a subject from influenza virus infection. In some embodiments, the influenza mRNA vaccine compositions described herein are used as a component of a seasonal and/or pandemic influenza vaccine or as part of an influenza vaccination regimen intended to confer long-lasting (multi-season) protection. In some embodiments, the influenza mRNA vaccine composition described herein are used to treat the symptoms of influenza infection.

In some embodiments, the subject is a non-human mammal. In some embodiments, the subject is a farm animal or a pet (e.g., a dog, a cat, a sheep, cattle, and/or a pig). In some embodiments, the subject is a non-human primate. In some embodiments, the subject is an avian (e.g., a chicken).

In some embodiments, the subject is a human. In certain embodiments, the subject is an adult, an adolescent, or an infant. In some embodiments, the human subject is younger than 6 months of age. In some embodiments, the human subject is 6 months of age or older, is 6 months through 35 months of age, is 36 months through 8 years of age, or 9 years of age or older. In some embodiments, the human subject is an elderly aged 55 years or older, such as 60 year of age or older, or 65 years of age or older. Also contemplated by the present invention are the administration of the composition and/or performance of the methods of treatment in-utero.

The invention is further defined with reference to the following numbered paragraphs:

1. A composition comprising at least three messenger RNAs (mRNAs), wherein the at least three mRNAs comprise an open reading frame (ORF) encoding a hemagglutinin (HA) antigen selected from the group consisting of:
(i) a first mRNA encoding an HA antigen of a first influenza A virus;
(ii) a second mRNA encoding an HA antigen of a second influenza A virus, wherein the first influenza A virus and the second influenza A virus are of different subtypes; and
(iii) a third mRNA encoding an HA antigen of a first influenza B virus,
   wherein the mRNA encoding the HA antigen of the influenza A virus is present in a different ratio (w/w) than the mRNA encoding the HA antigen of the influenza B virus.
   2. The composition of claim 1, further comprising a fourth mRNA encoding an HA antigen of a second influenza B virus, and wherein the first influenza B virus and the second influenza B virus are of different lineages.
   3. The composition of paragraph 1, wherein the first mRNA, the second mRNA, and the third mRNA are present in the ratio (w/w) of about 1:1:2.
   4. The composition of paragraph 1, wherein the first mRNA, the second mRNA, and the third mRNA are present in the ratio (w/w) of about 1:1:3.
   5. The composition of paragraph 1, wherein the first mRNA, the second mRNA, and the third mRNA are present in the ratio (w/w) of about 1:1:4.
   6. The composition of paragraph 1, wherein the first mRNA, the second mRNA, and the third mRNA are present in the ratio (w/w) of about 1:1:5.
   7. The composition of paragraph 1, wherein the first mRNA, the second mRNA, and the third mRNA are present in the ratio (w/w) of about 1:1:6.
   8. The composition of paragraph 1, wherein the first mRNA, the second mRNA, and the third mRNA are present in the ratio (w/w) of about 1:1:7.
   9. The composition of paragraph 1, wherein the first mRNA, the second mRNA, and the third mRNA are present in the ratio (w/w) of about 1:1:8.
   10. The composition of paragraph 1, wherein the first mRNA, the second mRNA, and the third mRNA are present in the ratio (w/w) of about 1:1:9.
   11. The composition of paragraph 1, wherein the first mRNA, the second mRNA, and the third mRNA are present in the ratio (w/w) of about 1:1:10.
   12. The composition of paragraph 1, wherein the first mRNA, the second mRNA, and the third mRNA, are present in the ratio (w/w) of about 1:1:2 to about 1:1:10.
   13. The composition of paragraph 2, wherein the first mRNA, the second mRNA, the third mRNA, and the fourth mRNA are present in the ratio (w/w) of about 1:1:2:2.
   14. The composition of paragraph 2, wherein the first mRNA, the second mRNA, the third mRNA, and the fourth mRNA are present in the ratio (w/w) of about 1:1:3:3.
   15. The composition of paragraph 2, wherein the first mRNA, the second mRNA, the third mRNA, and the fourth mRNA are present in the ratio (w/w) of about 1:1:4:4.
   16. The composition of paragraph 2, wherein the first mRNA, the second mRNA, the third mRNA, and the fourth mRNA are present in the ratio (w/w) of about 1:1:5:5.
   17. The composition of paragraph 2, wherein the first mRNA, the second mRNA, the third mRNA, and the fourth mRNA are present in the ratio (w/w) of about 1:1:6:6.
   18. The composition of paragraph 2, wherein the first mRNA, the second mRNA, the third mRNA, and the fourth mRNA are present in the ratio (w/w) of about 1:1:7:7.
   19. The composition of paragraph 2, wherein the first mRNA, the second mRNA, the third mRNA, and the fourth mRNA are present in the ratio (w/w) of about 1:1:8:8.
   20. The composition of paragraph 2, wherein the first mRNA, the second mRNA, the third mRNA, and the fourth mRNA are present in the ratio (w/w) of about 1:1:9:9.
   21. The composition of paragraph 2, wherein the first mRNA, the second mRNA, the third mRNA, and the fourth mRNA are present in the ratio (w/w) of about 1:1:10:10.
   22. The composition of paragraph 2, wherein the first mRNA, the second mRNA, the third mRNA, and the fourth mRNA are present in the ratio (w/w) of about 1:1:2:2 to about 1:1:10:10.
   23. The composition of any one of paragraphs 1-22, wherein the ratio is expressed in micrograms (μg).
   24. The composition of paragraph 13, wherein the first mRNA, the second mRNA, the third mRNA, and the fourth mRNA are present in a ratio (w/w) of about 32 micrograms of the first mRNA, to about 32 micrograms of the second mRNA, to about 64 micrograms of the third mRNA, to about 64 micrograms of the fourth mRNA.
   25. The composition of paragraph 15, wherein the first mRNA, the second mRNA, the third mRNA, and the fourth mRNA are present in a ratio (w/w) of about 16 micrograms of the first mRNA, to about 16 micrograms of the second mRNA, to about 64 micrograms of the third mRNA, to about 64 micrograms of the fourth mRNA.
   26. The composition of paragraph 17, wherein the first mRNA, the second mRNA, the third mRNA, and the fourth mRNA are present in a ratio (w/w) of about 16 micrograms of the first mRNA, to about 16 micrograms of the second mRNA, to about 96 micrograms of the third mRNA, to about 96 micrograms of the fourth mRNA.
   27. The composition of any one of paragraphs 13-23, wherein the composition comprises 130 micrograms of the mRNA in total.
   28. The composition of any one of paragraphs 13-23, wherein the composition comprises 160 micrograms of the mRNA in total.
   29. The composition of any one of paragraphs 13-23, wherein the composition comprises 200 micrograms of the mRNA in total.
   30. The composition of any one of paragraphs 13-23, wherein the composition comprises 224 micrograms of the mRNA in total.
   31. The composition of any one of paragraphs 13-23 wherein the composition comprises 130 micrograms to 224 micrograms of the mRNA in total.
   32. The composition of any one of the preceding paragraphs, wherein the first mRNA, the second mRNA, the third mRNA, and/or the fourth mRNA are not covalently linked to one another.
   33. The composition of any one of paragraphs 1-31, wherein one or more of the first mRNA, the second mRNA, the third mRNA, and/or the fourth mRNA are covalently linked to one another.
   34. The composition of any one of the preceding paragraphs, wherein the first mRNA, the second mRNA, the third mRNA, and/or the fourth mRNA are formulated into a LNP.
   35. The composition of paragraph 34, wherein the LNP comprises at least one cationic lipid.
   36. The composition of paragraph 35, wherein the cationic lipid is biodegradable.
   37. The composition of paragraph 35, wherein the cationic lipid is not biodegradable.
   38. The composition of paragraph 35, wherein the cationic lipid is cleavable.
   39. The composition of paragraph 35, wherein the cationic lipid is not cleavable.
   40. The composition of paragraph 35, wherein the cationic lipid is selected from the group consisting of OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315) and IM-001.
   41. The composition of paragraph 40, wherein the cationic lipid is cKK-E10.
   42. The composition of paragraph 40, wherein the cationic lipid is GL-HEPES-E3-E12-DS-4-E10.
   43. The composition of paragraph 40, wherein the cationic lipid is IM-001.
   44. The composition of any one of paragraphs 34-43, wherein the LNP further comprises a polyethylene glycol (PEG) conjugated (PEGylated) lipid, a cholesterol-based lipid, and a helper lipid.
   45. The composition of any one of paragraphs 34-44, wherein the LNP comprises:
a cationic lipid at a molar ratio of 35% to 55%;
a polyethylene glycol (PEG) conjugated (PEGylated) lipid at a molar ratio of 0.25% to 2.75%;
a cholesterol-based lipid at a molar ratio of 20% to 45%; and
a helper lipid at a molar ratio of 5% to 35%,
wherein all of the molar ratios are relative to the total lipid content of the LNP.
   46. The composition of paragraph 45, wherein the LNP comprises:
a cationic lipid at a molar ratio of 40%;
a PEGylated lipid at a molar ratio of 1.5%;
a cholesterol-based lipid at a molar ratio of 28.5%; and
a helper lipid at a molar ratio of 30%,
wherein all of the molar ratios are relative to the total lipid content of the LNP.
   47. The composition of any one of paragraphs 44-46, wherein the PEGylated lipid is dimyristoyl-PEG2000 (DMG-PEG2000) or 2-[(polyethylene glycol)-2000]-N,N-ditetradecylacetamide (ALC-0159).
   48. The composition of any one of paragraphs 44-47, wherein the cholesterol-based lipid is cholesterol.
   49. The composition of any one of paragraphs 44-48, wherein the helper lipid is 1,2-dioleoyl-SN-glycero-3-phosphoethanolamine (DOPE) or 1,2-distearoyl-sn-glycero-3-phosphocholine (DSPC).
   50. The composition of any one of paragraphs 34-49, wherein the LNP comprises:
GL-HEPES-E3-E12-DS-4-E10 at a molar ratio of 40%;
DMG-PEG2000 at a molar ratio of 1.5%;
cholesterol at a molar ratio of 28.5%; and
DOPE at a molar ratio of 30%,
wherein all of the molar ratios are relative to the total lipid content of the LNP.
   51. The composition of any one of paragraphs 34-49, wherein the LNP comprises:
cKK-E10 at a molar ratio of 40%;
DMG-PEG2000 at a molar ratio of 1.5%;
cholesterol at a molar ratio of 28.5%; and
DOPE at a molar ratio of 30%,
wherein all of the molar ratios are relative to the total lipid content of the LNP.
   52. The composition of any one of paragraphs 34-49, wherein the LNP comprises:
IM-001 at a molar ratio of 40%;
DMG-PEG2000 at a molar ratio of 1.5%;
cholesterol at a molar ratio of 28.5%; and
DOPE at a molar ratio of 30%,
wherein all of the molar ratios are relative to the total lipid content of the LNP.
   53. The composition of any one of paragraphs 34-52, wherein the LNP has an average diameter of 30 nm to 200 nm.
   54. The composition of paragraph 53, wherein the LNP has an average diameter of 80 nm to 150 nm.
   55. The composition of any one of the preceding paragraphs, wherein the first mRNA encodes an HA antigen of the influenza A subtype H1N1.
   56. The composition of any one of the preceding paragraphs, wherein the second mRNA encodes an HA antigen of the influenza A subtype H3N2.
   57. The composition of any one of the preceding paragraphs, wherein the third mRNA encodes an HA antigen of the influenza B Victoria-lineage strain.
   58. The composition of any one of paragraphs 2-57, wherein the fourth mRNA encodes an HA antigen of the influenza B Yamagata-lineage strain.
   59. The composition of any one of the preceding paragraphs, wherein at least one of the mRNAs comprises a codon-optimized ORF.
   60. The composition of any one of the preceding paragraphs, wherein at least one of the mRNAs comprises at least one 5′ untranslated region (5′ UTR), at least one 3′ untranslated region (3′ UTR), and at least one polyadenylation (poly(A)) sequence.
   61. The composition of any one of the preceding paragraphs, wherein at least one of the mRNAs comprises at least one chemical modification.
   62. The composition of any one of the preceding paragraphs, wherein at least 20%, at least 30%, at least 40%, at least 50%, at least 60%, at least 70%, at least 80%, at least 85%, at least 90%, at least 95%, or 100% of the uracil nucleotides in at least one of the mRNAs are chemically modified.
   63. The composition of any one of the preceding paragraphs, wherein at least 20%, at least 30%, at least 40%, at least 50%, at least 60%, at least 70%, at least 80%, at least 85%, at least 90%, at least 95%, or 100% of the uracil nucleotides in at least one of the ORFs are chemically modified.
   64. The composition of any one of paragraphs 50-52, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
   65. The composition of paragraph 64, wherein the chemical modification is selected from the group consisting of pseudouridine, N1-methylpseudouridine, 5-methylcytosine, 5-methoxyuridine, and a combination thereof.
   66. The composition of paragraph 65, wherein the chemical modification is N1-methylpseudouridine.
   67. A method comprising administering to a subject in need thereof the composition of any one of paragraphs 1-66.
   68. A method of eliciting an immune response to influenza A or protecting a subject against an influenza A infection, comprising administering to the subject the composition of any one of paragraphs 1-66.
   69. A method of eliciting an immune response to influenza B or protecting a subject against an influenza B infection, comprising administering to the subject the composition of any one of paragraphs 1-66.
   70. The method of paragraph 68, wherein the subject has a comparable serum concentration of neutralizing antibodies against influenza A after administration of the composition, relative to a subject that is administered a protein influenza A vaccine.
   71. The method of paragraph 69, wherein the subject has a comparable serum concentration of neutralizing antibodies against influenza B after administration of the composition, relative to a subject that is administered a protein influenza B vaccine.
   72. The method of paragraph 68, wherein the composition increases the serum concentration of neutralizing antibodies in a subject against influenza A.
   73. The method of paragraph 69, wherein the composition increases the serum concentration of neutralizing antibodies in a subject against influenza B.
   74. The method of paragraph 72, wherein the composition increases the serum concentration of neutralizing antibodies in a subject against influenza A H1N1 and/or influenza A H3N2.
   75. The method of paragraph 73, wherein the composition increases the serum concentration of neutralizing antibodies in a subject against influenza B Yamagata-lineage and/or Victoria-lineage.
   76. A composition of any one of paragraphs 1-66 for use in a method of eliciting an immune response to influenza A or protecting a subject against influenza A infection.
   77. A composition of any one of paragraphs 1-66 for use in a method of eliciting an immune response to influenza B or protecting a subject against influenza B infection.
   78. The use of the composition of any one of paragraphs 1-66 in the manufacture of a medicament for eliciting an immune response to influenza A or protecting a subject against influenza A infection.
   79. The use of the composition of any one of paragraphs 1-66 in the manufacture of a medicament for eliciting an immune response to influenza B or protecting a subject against influenza B infection.
   80. A composition comprising at least three messenger RNAs (mRNAs), wherein:
(i) a first mRNA encodes a hemagglutinin (HA) antigen of a first influenza A virus;
(ii) a second mRNA encodes an HA antigen of a second influenza A virus, wherein the first influenza A virus and the second influenza A virus are of different subtypes; and
(iii) a third mRNA encodes an HA antigen of a first influenza B virus,
   wherein the mRNA encoding the HA antigen of the influenza A virus is present in a different ratio (w/w) than the mRNA encoding the HA antigen of the influenza B virus, and wherein the first mRNA, the second mRNA, and the third mRNA are formulated into a lipid nanoparticle (LNP) comprising:
OF-02, cKK-E10, GL-HEPES-E3-E10-DS-3-E18-1, GL-HEPES-E3-E12-DS-4-E10, GL-HEPES-E3-E12-DS-3-E14, or IM-001 at a molar ratio of 40%;
DMG-PEG2000 at a molar ratio of 1.5%;
cholesterol at a molar ratio of 28.5%; and
DOPE at a molar ratio of 30%.
   81. The composition of paragraph 80, further comprising a fourth mRNA encoding an HA antigen of a second influenza B virus, and wherein the first influenza B virus and the second influenza B virus are of different lineages, and wherein the first mRNA, the second mRNA, the third mRNA, and the fourth mRNA are formulated into a lipid nanoparticle (LNP) comprising:
OF-02, cKK-E10, GL-HEPES-E3-E10-DS-3-E18-1, GL-HEPES-E3-E12-DS-4-E10, GL-HEPES-E3-E12-DS-3-E14, or IM-001 at a molar ratio of 40%;
DMG-PEG2000 at a molar ratio of 1.5%;
cholesterol at a molar ratio of 28.5%; and
DOPE at a molar ratio of 30%.
   82. The composition of paragraph 80, wherein the first mRNA, the second mRNA, and the third mRNA are present in the ratio (w/w) of about 1:1:2.
   83. The composition of paragraph 80, wherein the first mRNA, the second mRNA, and the third mRNA are present in the ratio (w/w) of about 1:1:3.
   84. The composition of paragraph 80, wherein the first mRNA, the second mRNA, and the third mRNA are present in the ratio (w/w) of about 1:1:4.
   85. The composition of paragraph 80, wherein the first mRNA, the second mRNA, and the third mRNA are present in the ratio (w/w) of about 1:1:5.
   86. The composition of paragraph 80, wherein the first mRNA, the second mRNA, and the third mRNA are present in the ratio (w/w) of about 1:1:6.
   87. The composition of paragraph 80, wherein the first mRNA, the second mRNA, and the third mRNA are present in the ratio (w/w) of about 1:1:7.
   88. The composition of paragraph 80, wherein the first mRNA, the second mRNA, and the third mRNA are present in the ratio (w/w) of about 1:1:8.
   89. The composition of paragraph 80, wherein the first mRNA, the second mRNA, and the third mRNA are present in the ratio (w/w) of about 1:1:9.
   90. The composition of paragraph 80, wherein the first mRNA, the second mRNA, and the third mRNA are present in the ratio (w/w) of about 1:1:10.
   91. The composition of paragraph 80, wherein the first mRNA, the second mRNA, and the third mRNA, are present in the ratio (w/w) of about 1:1:2 to about 1:1:10.
   92. The composition of paragraph 81, wherein the first mRNA, the second mRNA, the third mRNA, and the fourth mRNA are present in the ratio (w/w) of about 1:1:2:2.
   93. The composition of paragraph 81, wherein the first mRNA, the second mRNA, the third mRNA, and the fourth mRNA are present in the ratio (w/w) of about 1:1:3:3.
   94. The composition of paragraph 81, wherein the first mRNA, the second mRNA, the third mRNA, and the fourth mRNA are present in the ratio (w/w) of about 1:1:4:4.
   95. The composition of paragraph 81, wherein the first mRNA, the second mRNA, the third mRNA, and the fourth mRNA are present in the ratio (w/w) of about 1:1:5:5.
   96. The composition of paragraph 81, wherein the first mRNA, the second mRNA, the third mRNA, and the fourth mRNA are present in the ratio (w/w) of about 1:1:6:6.
   97. The composition of paragraph 81, wherein the first mRNA, the second mRNA, the third mRNA, and the fourth mRNA are present in the ratio (w/w) of about 1:1:7:7.
   98. The composition of paragraph 81, wherein the first mRNA, the second mRNA, the third mRNA, and the fourth mRNA are present in the ratio (w/w) of about 1:1:8:8.
   99. The composition of paragraph 81, wherein the first mRNA, the second mRNA, the third mRNA, and the fourth mRNA are present in the ratio (w/w) of about 1:1:9:9.
   100. The composition of paragraph 81, wherein the first mRNA, the second mRNA, the third mRNA, and the fourth mRNA are present in the ratio (w/w) of about 1:1:10:10.
   101. The composition of paragraph 81, wherein the first mRNA, the second mRNA, the third mRNA, and the fourth mRNA are present in the ratio (w/w) of about 1:1:2:2 to about 1:1:10:10.
   102. The composition of any one of paragraphs 80-101, wherein the ratio is expressed in micrograms (μg).
   103. The composition of paragraph 92, wherein the first mRNA, the second mRNA, the third mRNA, and the fourth mRNA are present in a ratio (w/w) of about 32 micrograms of the first mRNA, to about 32 micrograms of the second mRNA, to about 64 micrograms of the third mRNA, to about 64 micrograms of the fourth mRNA.
   104. The composition of paragraph 94, wherein the first mRNA, the second mRNA, the third mRNA, and the fourth mRNA are present in a ratio (w/w) of about 16 micrograms of the first mRNA, to about 16 micrograms of the second mRNA, to about 64 micrograms of the third mRNA, to about 64 micrograms of the fourth mRNA.
   105. The composition of paragraph 96, wherein the first mRNA, the second mRNA, the third mRNA, and the fourth mRNA are present in a ratio (w/w) of about 16 micrograms of the first mRNA, to about 16 micrograms of the second mRNA, to about 96 micrograms of the third mRNA, to about 96 micrograms of the fourth mRNA.
   106. The composition of any one of paragraphs 92-102, wherein the composition comprises 130 micrograms of the mRNA in total.
   107. The composition of any one of paragraphs 92-102, wherein the composition comprises 160 micrograms of the mRNA in total.
   108. The composition of any one of paragraphs 92-102, wherein the composition comprises 200 micrograms of the mRNA in total.
   109. The composition of any one of paragraphs 92-102, wherein the composition comprises 224 micrograms of the mRNA in total.
   110. The composition of any one of paragraphs 92-102, wherein the composition comprises 130 micrograms to 224 micrograms of the mRNA in total.
   111. The composition of any one of paragraphs 80-110, wherein at least one of the mRNAs comprises a codon-optimized open reading frame (ORF).
   112. The composition of any one of paragraphs 80-111, wherein at least one of the mRNAs comprises at least one 5′ untranslated region (5′ UTR), at least one 3′ untranslated region (3′ UTR), and at least one polyadenylation (poly(A)) sequence.
   113. The composition of any one of paragraphs 80-112, wherein at least one of the mRNAs comprises at least one chemical modification.
   114. The composition of any one of paragraphs 80-113, wherein the first mRNA, the second mRNA, the third mRNA, and/or the fourth mRNA are not covalently linked to one another.
   115. The composition of any one of paragraphs 80-113, wherein one or more of the first mRNA, the second mRNA, the third mRNA, and/or the fourth mRNA are covalently linked to one another.
   116. The composition of any one of paragraphs 80-115, wherein the LNP has an average diameter of 30 nm to 200 nm.
   117. The composition of paragraph 116, wherein the LNP has an average diameter of 80 nm to 150 nm.
   118. The composition of any one of paragraphs 80-117, wherein the first mRNA encodes an HA antigen of the influenza A subtype H1N1.
   119. The composition of any one of paragraphs 80-118, wherein the second mRNA encodes an HA antigen of the influenza A subtype H3N2.
   120. The composition of any one of paragraphs 80-119, wherein the third mRNA encodes an HA antigen of the influenza B Victoria-lineage strain.
   121. The composition of any one of paragraphs 81-120, wherein the fourth mRNA encodes an HA antigen of the influenza B Yamagata-lineage strain.
   122. A method comprising administering to a subject in need thereof the composition of any one of paragraphs 80-121.
   123. A method of eliciting an immune response to influenza A or protecting a subject against an influenza A infection, comprising administering to the subject the composition of any one of paragraphs 80-121.
   124. A method of eliciting an immune response to influenza B or protecting a subject against an influenza B infection, comprising administering to the subject the composition of any one of paragraphs 80-121.
   125. The method of paragraph 123, wherein the subject has a comparable serum concentration of neutralizing antibodies against influenza A after administration of the composition, relative to a subject that is administered a protein influenza A vaccine.
   126. The method of paragraph 124, wherein the subject has a comparable serum concentration of neutralizing antibodies against influenza B after administration of the composition, relative to a subject that is administered a protein influenza B vaccine.
   127. The method of paragraph 123, wherein the composition increases the serum concentration of neutralizing antibodies in a subject against influenza A.
   128. The method of paragraph 124, wherein the composition increases the serum concentration of neutralizing antibodies in a subject against influenza B.
   129. The method of paragraph 123, wherein the composition increases the serum concentration of neutralizing antibodies in a subject against influenza A H1N1 and/or influenza A H3N2.
   130. The method of paragraph 124, wherein the composition increases the serum concentration of neutralizing antibodies in a subject against influenza B Yamagata-lineage and/or Victoria-lineage.
   131. A composition of any one of paragraphs 80-121 for use in a method of eliciting an immune response to influenza A or protecting a subject against influenza A infection.
   132. A composition of any one of paragraphs 80-121 for use in a method of eliciting an immune response to influenza B or protecting a subject against influenza B infection.
   133. The use of the composition of any one of paragraphs 80-121 in the manufacture of a medicament for eliciting an immune response to influenza A or protecting a subject against influenza A infection.
   134. The use of the composition of any one of paragraphs 80-121 in the manufacture of a medicament for eliciting an immune response to influenza B or protecting a subject against influenza B infection.
   135. A method comprising administering to a human subject a composition comprising at least three messenger RNAs (mRNAs), wherein:
(i) a first mRNA encodes a hemagglutinin (HA) antigen of a first influenza A virus;
(ii) a second mRNA encodes an HA antigen of a second influenza A virus, wherein the first influenza A virus and the second influenza A virus are of different subtypes; and
(iii) a third mRNA encodes an HA antigen of a first influenza B virus,
   wherein the mRNA encoding the HA antigen of the influenza A virus is present in a different ratio (w/w) than the mRNA encoding the HA antigen of the influenza B virus, and wherein the first mRNA, the second mRNA, and the third mRNA, are formulated into a lipid nanoparticle (LNP) comprising:
OF-02, cKK-E10, GL-HEPES-E3-E10-DS-3-E18-1, GL-HEPES-E3-E12-DS-4-E10, GL-HEPES-E3-E12-DS-3-E14, or IM-001 at a molar ratio of 40%;
DMG-PEG2000 at a molar ratio of 1.5%;
cholesterol at a molar ratio of 28.5%; and
DOPE at a molar ratio of 30%.
   136. The method of paragraph 135, wherein the composition comprises a fourth mRNA that encodes an HA antigen of a second influenza B virus, wherein the first influenza B virus and the second influenza B virus are of different lineages, and wherein the first mRNA, the second mRNA, the third mRNA, and the fourth mRNA are formulated into a lipid nanoparticle (LNP) comprising:
OF-02, cKK-E10, GL-HEPES-E3-E10-DS-3-E18-1, GL-HEPES-E3-E12-DS-4-E10, GL-HEPES-E3-E12-DS-3-E14, or IM-001 at a molar ratio of 40%;
DMG-PEG2000 at a molar ratio of 1.5%;
cholesterol at a molar ratio of 28.5%; and
DOPE at a molar ratio of 30%.
   137. The composition of paragraph 135, wherein the first mRNA, the second mRNA, and the third mRNA are present in the ratio (w/w) of about 1:1:2.
   138. The composition of paragraph 135, wherein the first mRNA, the second mRNA, and the third mRNA are present in the ratio (w/w) of about 1:1:3.
   139. The composition of paragraph 135, wherein the first mRNA, the second mRNA, and the third mRNA are present in the ratio (w/w) of about 1:1:4.
   140. The composition of paragraph 135, wherein the first mRNA, the second mRNA, and the third mRNA are present in the ratio (w/w) of about 1:1:5.
   141. The composition of paragraph 135, wherein the first mRNA, the second mRNA, and the third mRNA are present in the ratio (w/w) of about 1:1:6.
   142. The composition of paragraph 135, wherein the first mRNA, the second mRNA, and the third mRNA are present in the ratio (w/w) of about 1:1:7.
   143. The composition of paragraph 135, wherein the first mRNA, the second mRNA, and the third mRNA are present in the ratio (w/w) of about 1:1:8.
   144. The composition of paragraph 135, wherein the first mRNA, the second mRNA, and the third mRNA are present in the ratio (w/w) of about 1:1:9.
   145. The composition of paragraph 135, wherein the first mRNA, the second mRNA, and the third mRNA are present in the ratio (w/w) of about 1:1:10.
   146. The composition of paragraph 135, wherein the first mRNA, the second mRNA, and the third mRNA, are present in the ratio (w/w) of about 1:1:2 to about 1:1:10.
   147. The method of paragraph 136, wherein the first mRNA, the second mRNA, the third mRNA, and the fourth mRNA are present in the ratio (w/w) of about 1:1:2:2.
   148. The method of paragraph 136, wherein the first mRNA, the second mRNA, the third mRNA, and the fourth mRNA are present in the ratio (w/w) of about 1:1:3:3.
   149. The method of paragraph 136, wherein the first mRNA, the second mRNA, the third mRNA, and the fourth mRNA are present in the ratio (w/w) of about 1:1:4:4.
   150. The method of paragraph 136, wherein the first mRNA, the second mRNA, the third mRNA, and the fourth mRNA are present in the ratio (w/w) of about 1:1:5:5.
   151. The method of paragraph 136, wherein the first mRNA, the second mRNA, the third mRNA, and the fourth mRNA are present in the ratio (w/w) of about 1:1:6:6.
   152. The composition of paragraph 136, wherein the first mRNA, the second mRNA, the third mRNA, and the fourth mRNA are present in the ratio (w/w) of about 1:1:7:7.
   153. The composition of paragraph 136, wherein the first mRNA, the second mRNA, the third mRNA, and the fourth mRNA are present in the ratio (w/w) of about 1:1:8:8.
   154. The composition of paragraph 136, wherein the first mRNA, the second mRNA, the third mRNA, and the fourth mRNA are present in the ratio (w/w) of about 1:1:9:9.
   155. The composition of paragraph 136, wherein the first mRNA, the second mRNA, the third mRNA, and the fourth mRNA are present in the ratio (w/w) of about 1:1:10:10.
   156. The composition of paragraph 136, wherein the first mRNA, the second mRNA, the third mRNA, and the fourth mRNA are present in the ratio (w/w) of about 1:1:2:2 to about 1:1:10:10.
   157. The method of one of paragraphs 135-156, wherein the ratio is expressed in micrograms (μg).
   158. The method of paragraph 147, wherein the first mRNA, the second mRNA, the third mRNA, and the fourth mRNA are present in a ratio (w/w) of about 32 micrograms of the first mRNA, to about 32 micrograms of the second mRNA, to about 64 micrograms of the third mRNA, to about 64 micrograms of the fourth mRNA.
   159. The method of paragraph 149, wherein the first mRNA, the second mRNA, the third mRNA, and the fourth mRNA are present in a ratio (w/w) of about 16 micrograms of the first mRNA, to about 16 micrograms of the second mRNA, to about 64 micrograms of the third mRNA, to about 64 micrograms of the fourth mRNA.
   160. The method of paragraph 151, wherein the first mRNA, the second mRNA, the third mRNA, and the fourth mRNA are present in a ratio (w/w) of about 16 micrograms of the first mRNA, to about 16 micrograms of the second mRNA, to about 96 micrograms of the third mRNA, to about 96 micrograms of the fourth mRNA.
   161. The method of any one of paragraphs 147-157, wherein the composition comprises 130 micrograms of the mRNA in total.
   162. The method of any one of paragraphs 147-157, wherein the composition comprises 160 micrograms of the mRNA in total.
   163. The method of any one of paragraphs 147-157, wherein the composition comprises 200 micrograms of the mRNA in total.
   164. The method of any one of paragraphs 147-157, wherein the composition comprises 224 micrograms of the mRNA in total.
   165. The method of any one of paragraphs 147-157, wherein the composition comprises 130 micrograms to 224 micrograms of the mRNA in total.
   166. The method of any one of paragraphs 135-165, wherein at least one of the mRNAs comprises a codon-optimized open reading frame (ORF).
   167. The method of any one of paragraphs 135-166, wherein at least one of the mRNAs comprises at least one 5′ untranslated region (5′ UTR), at least one 3′ untranslated region (3′ UTR), and at least one polyadenylation (poly(A)) sequence.
   168. The method of any one of paragraphs 135-167, wherein at least one of the mRNAs comprises at least one chemical modification.
   169. The method of any one of paragraphs 135-168, wherein the first mRNA, the second mRNA, the third mRNA, and/or the fourth mRNA are not covalently linked to one another.
   170. The method of any one of paragraphs 135-168, wherein one or more of the first mRNA, the second mRNA, the third mRNA, and/or the fourth mRNA are covalently linked to one another.
   171. The method of any one of paragraphs 135-170, wherein the LNP has an average diameter of 30 nm to 200 nm.
   172. The method of paragraph 171, wherein the LNP has an average diameter of 80 nm to 150 nm.
   173. The method of any one of paragraphs 135-172, wherein the first mRNA encodes an HA antigen of the influenza A subtype H1N1.
   174. The method of any one of paragraphs 135-173, wherein the second mRNA encodes an HA antigen of the influenza A subtype H3N2.
   175. The method of any one of paragraphs 135-174, wherein the third mRNA encodes an HA antigen of the influenza B Victoria-lineage strain.
   176. The method of any one of paragraphs 136-175, wherein the fourth mRNA encodes an HA antigen of the influenza B Yamagata-lineage strain.
   177. The method of any one of paragraphs 135-176, wherein the composition is administered in an effective amount to elicit an immune response to influenza A or to protect a subject against an influenza A infection.
   178. The method of any one of paragraphs 135-176, wherein the composition is administered in an effective amount to elicit an immune response to influenza B or to protect a subject against an influenza B infection.
   179. The method of any one of paragraphs 135-176, wherein the subject has a comparable serum concentration of neutralizing antibodies against influenza A after administration of the composition, relative to a subject that is administered a protein influenza A vaccine.
   180. The method of any one of paragraphs 135-176, wherein the subject has a comparable serum concentration of neutralizing antibodies against influenza B after administration of the composition, relative to a subject that is administered a protein influenza B vaccine.
   181. The method of any one of paragraphs 135-176, wherein the composition increases the serum concentration of neutralizing antibodies in a subject against influenza A.
   182. The method of any one of paragraphs 135-176, wherein the composition increases the serum concentration of neutralizing antibodies in a subject against influenza B.
   183. The method of paragraph 181, wherein the composition increases the serum concentration of neutralizing antibodies in a subject against influenza A H1N1 and/or influenza A H3N2.
   184. The method of paragraph 182, wherein the composition increases the serum concentration of neutralizing antibodies in a subject against influenza B Yamagata-lineage and/or Victoria-lineage.
   185. A composition comprising at least three messenger RNAs (mRNAs), wherein:
(i) a first mRNA encodes a hemagglutinin (HA) antigen of a first influenza A virus;
(ii) a second mRNA encodes an HA antigen of a second influenza A virus, wherein the first influenza A virus and the second influenza A virus are of different subtypes; and
(iii) a third mRNA encodes an HA antigen of a first influenza B virus,
   wherein the first mRNA, the second mRNA, and the third mRNA are formulated into a lipid nanoparticle (LNP) comprising IM-001.
   186. The composition of paragraph 185, further comprising a fourth mRNA encoding an HA antigen of a second influenza B virus, and wherein the first influenza B virus and the second influenza B virus are of different lineages, and wherein the first mRNA, the second mRNA, the third mRNA, and the fourth mRNA are formulated into a LNP comprising IM-001.
   187. The composition of paragraph 185, wherein the first mRNA, the second mRNA, and the third mRNA are present in the ratio (w/w) of about 1:1:2.
   188. The composition of paragraph 185, wherein the first mRNA, the second mRNA, and the third mRNA are present in the ratio (w/w) of about 1:1:3.
   189. The composition of paragraph 185, wherein the first mRNA, the second mRNA, and the third mRNA are present in the ratio (w/w) of about 1:1:4.
   190. The composition of paragraph 185, wherein the first mRNA, the second mRNA, and the third mRNA are present in the ratio (w/w) of about 1:1:5.
   191. The composition of paragraph 185, wherein the first mRNA, the second mRNA, and the third mRNA are present in the ratio (w/w) of about 1:1:6.
   192. The composition of paragraph 185, wherein the first mRNA, the second mRNA, and the third mRNA are present in the ratio (w/w) of about 1:1:7.
   193. The composition of paragraph 185, wherein the first mRNA, the second mRNA, and the third mRNA are present in the ratio (w/w) of about 1:1:8.
   194. The composition of paragraph 185, wherein the first mRNA, the second mRNA, and the third mRNA are present in the ratio (w/w) of about 1:1:9.
   195. The composition of paragraph 185, wherein the first mRNA, the second mRNA, and the third mRNA are present in the ratio (w/w) of about 1:1:10.
   196. The composition of paragraph 185, wherein the first mRNA, the second mRNA, and the third mRNA, are present in the ratio (w/w) of about 1:1:2 to about 1:1:10.
   197. The composition of paragraph 186, wherein the first mRNA, the second mRNA, the third mRNA, and the fourth mRNA are present in the ratio (w/w) of about 1:1:2:2.
   198. The composition of paragraph 186, wherein the first mRNA, the second mRNA, the third mRNA, and the fourth mRNA are present in the ratio (w/w) of about 1:1:3:3.
   199. The composition of paragraph 186, wherein the first mRNA, the second mRNA, the third mRNA, and the fourth mRNA are present in the ratio (w/w) of about 1:1:4:4.
   200. The composition of paragraph 186, wherein the first mRNA, the second mRNA, the third mRNA, and the fourth mRNA are present in the ratio (w/w) of about 1:1:5:5.
   201. The composition of paragraph 186, wherein the first mRNA, the second mRNA, the third mRNA, and the fourth mRNA are present in the ratio (w/w) of about 1:1:6:6.
   202. The composition of paragraph 186, wherein the first mRNA, the second mRNA, the third mRNA, and the fourth mRNA are present in the ratio (w/w) of about 1:1:7:7.
   203. The composition of paragraph 186, wherein the first mRNA, the second mRNA, the third mRNA, and the fourth mRNA are present in the ratio (w/w) of about 1:1:8:8.
   204. The composition of paragraph 186, wherein the first mRNA, the second mRNA, the third mRNA, and the fourth mRNA are present in the ratio (w/w) of about 1:1:9:9.
   205. The composition of paragraph 186, wherein the first mRNA, the second mRNA, the third mRNA, and the fourth mRNA are present in the ratio (w/w) of about 1:1:10:10.
   206. The composition of paragraph 186, wherein the first mRNA, the second mRNA, the third mRNA, and the fourth mRNA are present in the ratio (w/w) of about 1:1:2:2 to about 1:1:10:10.
   207. The composition of any one of paragraphs 187-205, wherein the ratio is expressed in micrograms (μg).
   208. The composition of paragraph 197, wherein the first mRNA, the second mRNA, the third mRNA, and the fourth mRNA are present in a ratio (w/w) of about 32 micrograms of the first mRNA, to about 32 micrograms of the second mRNA, to about 64 micrograms of the third mRNA, to about 64 micrograms of the fourth mRNA.
   209. The composition of paragraph 199, wherein the first mRNA, the second mRNA, the third mRNA, and the fourth mRNA are present in a ratio (w/w) of about 16 micrograms of the first mRNA, to about 16 micrograms of the second mRNA, to about 64 micrograms of the third mRNA, to about 64 micrograms of the fourth mRNA.
   210. The composition of paragraph 201, wherein the first mRNA, the second mRNA, the third mRNA, and the fourth mRNA are present in a ratio (w/w) of about 16 micrograms of the first mRNA, to about 16 micrograms of the second mRNA, to about 96 micrograms of the third mRNA, to about 96 micrograms of the fourth mRNA.
   211. The composition of any one of paragraphs 187-207, wherein the composition comprises 130 micrograms of the mRNA in total.
   212. The composition of any one of paragraphs 187-207, wherein the composition comprises 160 micrograms of the mRNA in total.
   213. The composition of any one of paragraphs 187-207, wherein the composition comprises 200 micrograms of the mRNA in total.
   214. The composition of any one of paragraphs 187-207, wherein the composition comprises 224 micrograms of the mRNA in total.
   215. The composition of any one of paragraphs 187-207, wherein the composition comprises 130 micrograms to 224 micrograms of the mRNA in total.
   216. The composition of any one of the preceding paragraphs, wherein the first mRNA, the second mRNA, the third mRNA, and/or the fourth mRNA are not covalently linked to one another.
   217. The composition of any one of paragraphs 185-215, wherein one or more of the first mRNA, the second mRNA, the third mRNA, and/or the fourth mRNA are covalently linked to one another.
   218. The composition of any one of the preceding paragraphs, wherein the LNP further comprises a polyethylene glycol (PEG) conjugated (PEGylated) lipid, a cholesterol-based lipid, and a helper lipid.
   219. The composition of any one of the preceding paragraphs, wherein the LNP comprises:
IM-001 at a molar ratio of 35% to 55%;
a polyethylene glycol (PEG) conjugated (PEGylated) lipid at a molar ratio of 0.25% to 2.75%;
a cholesterol-based lipid at a molar ratio of 20% to 45%; and
a helper lipid at a molar ratio of 5% to 35%,
wherein all of the molar ratios are relative to the total lipid content of the LNP.
   220. The composition of paragraph 219, wherein the LNP comprises:
IM-001 at a molar ratio of 40%;
a PEGylated lipid at a molar ratio of 1.5%;
a cholesterol-based lipid at a molar ratio of 28.5%; and
a helper lipid at a molar ratio of 30%,
wherein all of the molar ratios are relative to the total lipid content of the LNP.
   221. The composition of any one of paragraphs 218-220, wherein the PEGylated lipid is dimyristoyl-PEG2000 (DMG-PEG2000) or 2-[(polyethylene glycol)-2000]-N,N-ditetradecylacetamide (ALC-0159).
   222. The composition of any one of paragraphs 218-221, wherein the cholesterol-based lipid is cholesterol.
   223. The composition of any one of paragraphs 218-222, wherein the helper lipid is 1,2-dioleoyl-SN-glycero-3-phosphoethanolamine (DOPE) or 1,2-distearoyl-sn-glycero-3-phosphocholine (DSPC).
   224. The composition of any one of the preceding paragraphs, wherein the LNP has an average diameter of 30 nm to 200 nm.
   225. The composition of paragraph 224, wherein the LNP has an average diameter of 80 nm to 150 nm.
   226. The composition of any one of paragraphs 185-225, wherein the first mRNA encodes an HA antigen of the influenza A subtype H1N1.
   227. The composition of any one of paragraphs 185-226, wherein the second mRNA encodes an HA antigen of the influenza A subtype H3N2.
   228. The composition of any one of paragraphs 185-227, wherein the third mRNA encodes an HA antigen of the influenza B Victoria-lineage strain.
   229. The composition of any one of paragraphs 186-229, wherein the fourth mRNA encodes an HA antigen of the influenza B Yamagata-lineage strain.
   230. The composition of any one of paragraphs 185-229, wherein at least one of the mRNAs comprises a codon-optimized ORF.
   231. The composition of any one of paragraphs 185-230, wherein at least one of the mRNAs comprises at least one 5′ untranslated region (5′ UTR), at least one 3′ untranslated region (3′ UTR), and at least one polyadenylation (poly(A)) sequence.
   232. The composition of any one of paragraphs 185-231, wherein at least one of the mRNAs comprises at least one chemical modification.
   233. The composition of any one of paragraphs 185-232, wherein at least 20%, at least 30%, at least 40%, at least 50%, at least 60%, at least 70%, at least 80%, at least 85%, at least 90%, at least 95%, or 100% of the uracil nucleotides in at least one of the mRNAs are chemically modified.
   234. The composition of any one of paragraphs 185-233, wherein at least 20%, at least 30%, at least 40%, at least 50%, at least 60%, at least 70%, at least 80%, at least 85%, at least 90%, at least 95%, or 100% of the uracil nucleotides in at least one of the ORFs are chemically modified.
   235. The composition of any one of paragraphs 232-234, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
   236. The composition of paragraph 235, wherein the chemical modification is selected from the group consisting of pseudouridine, N1-methylpseudouridine, 5-methylcytosine, 5-methoxyuridine, and a combination thereof.
   237. The composition of paragraph 236, wherein the chemical modification is N1-methylpseudouridine.
   238. A method comprising administering to a subject in need thereof the composition of any one of paragraphs 185-237.
   239. A method of eliciting an immune response to influenza A or protecting a subject against an influenza A infection, comprising administering to the subject the composition of any one of paragraphs 185-237.
   240. A method of eliciting an immune response to influenza B or protecting a subject against an influenza B infection, comprising administering to the subject the composition of any one of paragraphs 185-237.
   241. The method of paragraph 239, wherein the subject has a comparable serum concentration of neutralizing antibodies against influenza A after administration of the composition, relative to a subject that is administered a protein influenza A vaccine.
   242. The method of paragraph 240, wherein the subject has a comparable serum concentration of neutralizing antibodies against influenza B after administration of the composition, relative to a subject that is administered a protein influenza B vaccine.
   243. The method of paragraph 239, wherein the composition increases the serum concentration of neutralizing antibodies in a subject against influenza A.
   244. The method of paragraph 240, wherein the composition increases the serum concentration of neutralizing antibodies in a subject against influenza B.
   245. The method of paragraph 243, wherein the composition increases the serum concentration of neutralizing antibodies in a subject against influenza A H1N1 and/or influenza A H3N2.
   246. The method of paragraph 244, wherein the composition increases the serum concentration of neutralizing antibodies in a subject against influenza B Yamagata-lineage and/or Victoria-lineage.
   247. A composition of any one of paragraphs 185-237 for use in a method of eliciting an immune response to influenza A or protecting a subject against influenza A infection.
   248. A composition of any one of paragraphs 185-237 for use in a method of eliciting an immune response to influenza B or protecting a subject against influenza B infection.
   249. The use of the composition of any one of paragraphs 185-237 in the manufacture of a medicament for eliciting an immune response to influenza A or protecting a subject against influenza A infection.
   250. The use of the composition of any one of paragraphs 185-237 in the manufacture of a medicament for eliciting an immune response to influenza B or protecting a subject against influenza B infection.

### EXAMPLES

### Example 1:1:1:1:1 Combination Influenza mRNA Vaccine Formulations: Immunogenicity in Mice

This Example describes experiments in which monovalent and quadrivalent mRNA-LNP vaccine formulations containing mRNA encoding the HA sequences of two influenza A strains (i.e. A/H1N1, A/H3N2) and two influenza B strains (i.e. B/Yamagata-lineage, and B/Victoria-lineage) in a 1:1:1:1 ratio were evaluated for potency. Immunogenicity of three quadrivalent modified mRNA influenza vaccines encoding the HA sequences of two influenza A strains (i.e. A/H1N1, A/H3N2) and two influenza B strains (i.e. B/Yamagata-lineage, and B/Victoria-lineage) in a 1:1:1:1 ratio, and encapsulated in an LNP formulation comprising: cationic lipid ckk-E10 at a molar ratio of 40%; DMG-PEG2000 at a molar ratio of 1.5%; cholesterol at a molar ratio of 28.5%; and DOPE at a molar ratio of 30%, 40 micrograms of mRNA encoding HA-1 (A/Michigan/45/2015), HA-3 (A/Singapore/INFIMH160019/2016), HA-B Victoria (B/Maryland/15/2016 BX69A) and HA-B Yamagata (B/Phuket/3073/2013), either singly or together (40 micrograms of each mRNA formulation co-mixed before administration), were administered to BALB/c mice in a two-dose regimen 3 weeks apart. Samples were taken at day 1, 20, 22 and 36. To measure functional antibodies, hemagglutination inhibition (HAI) response was evaluated against the homologous strains. The results for day 36 samples are shown in Table 1 below.

TABLE 1
HAI GMT titers in mice

| | H1 HAI | H3 HAI | BVic HAI | BYam HAI |
|---|---|---|---|---|
| | GMT | GMT | GMT | GMT |
| Vaccine | A/Mich/15 | A/Sing/16 | Maryland | Phuket |
| 40 μg H1 A/Mich/15 | 1016 | — | — | — |
| 40 μg H3 A/Sing/16 | — | 285 | — | — |
| 40 μg BVic (Mary) | — | — | 28 | — |
| 40 μg BYam | — | — | — | 57 |
| (Phuket) | | | | |
| 40 μg A/Mich/15 | 1810 | 160 | 36 | 50 |
| 40 μg A/Sing/16 | | | | |
| 40 μg BVic (Mary) | | | | |
| 40 μg BYam | | | | |
| (Phuket) | | | | |

No significant differences were seen between the monovalent and quadrivalent vaccines in terms of the immune responses to any of the four antigens. However, the HAI GMTs for the B strains were all at the limit of detection, indicating a reduced immune response compared to the A strains.

### Example 2:1:1:1:1 Combination Influenza mRNA Vaccine Formulations in Human Clinical Trials

Influenza pandemics can occur when a novel influenza virus emerges in the human population. Such pandemics remain a major threat to public health, requiring vigilant attention and preparedness with countermeasures to be used in the event of sustained human-to-human spread of the virus. In the experiments described in this Example, three Phase I/II studies were designed to assess the safety and immunogenicity of three quadrivalent modified mRNA influenza vaccines encoding the HA sequences of two influenza A strains (i.e. A/H1N1, A/H3N2) and two influenza B strains (i.e. B/Yamagata-lineage, and B/Victoria-lineage) in a 1:1:1:1 ratio, and encapsulated in an LNP formulation comprising one of the three cationic lipids: GL-HEPES-E3-E12-DS-4-E10 (clinical trial NCT05624606), ckk-E10 (clinical trial NCT05553301), or OF-02 (clinical trial NCT05650554). The composition of the LNPs was as follows: cationic lipid at a molar ratio of 40%; DMG-PEG2000 at a molar ratio of 1.5%; cholesterol at a molar ratio of 28.5%; and DOPE at a molar ratio of 30%.

Clinical trial NCT05624606 evaluates the safety and immunogenicity of the quadrivalent influenza mRNA vaccine MRT5410, administered as a single intramuscular injection at three dose levels (i.e. low, medium, high) in adults 18 years of age and older, and compared to the following active controls: (1) Fluzone Quadrivalent®, a standard does quadrivalent inactivated influenza vaccine and a comparator for study participants 18-64 years of age and ≥65 years of age; (2) Flublok Quadrivalent®, a quadrivalent recombinant influenza vaccine and a comparator for study participants 18-64 years of age and ≥65 years of age; and (3) Fluzone High-Dose Quadrivalent®, a high dose quadrivalent inactivated influenza vaccine and a comparator for study participants ≥65 years of age only.

Clinical trial NCT05553301 evaluates the safety and immunogenicity of the quadrivalent influenza mRNA vaccine MRT5407, administered as a single intramuscular injection at two dose levels (i.e. dose level 1, dose level 2), in adults 18 years of age and older, and compared to the following active controls: (1) Fluzone Quadrivalent®, a comparator for study participants 18-64 years of age and ≥65 years of age; (2) Flublok Quadrivalent®, a comparator for study participants 18-64 years of age and ≥65 years of age; and (3) Fluzone High-Dose Quadrivalent®, a comparator for study participants ≥65 years of age only.

Clinical trial NCT05650554 evaluates the safety and immunogenicity of the quadrivalent influenza mRNA vaccine MRT5413, administered as a single intramuscular injection at three dose levels (i.e. low, medium, high), in adults 18 years of age and older, and compared to the following active controls: (1) Fluzone Quadrivalent®, a comparator for study participants 18-64 years of age and ≥65 years of age; (2) Flublok Quadrivalent®, a comparator for study participants 18-64 years of age and >65 years of age; and (3) Fluzone High-Dose Quadrivalent®, a comparator for study participants ≥65 years of age only.

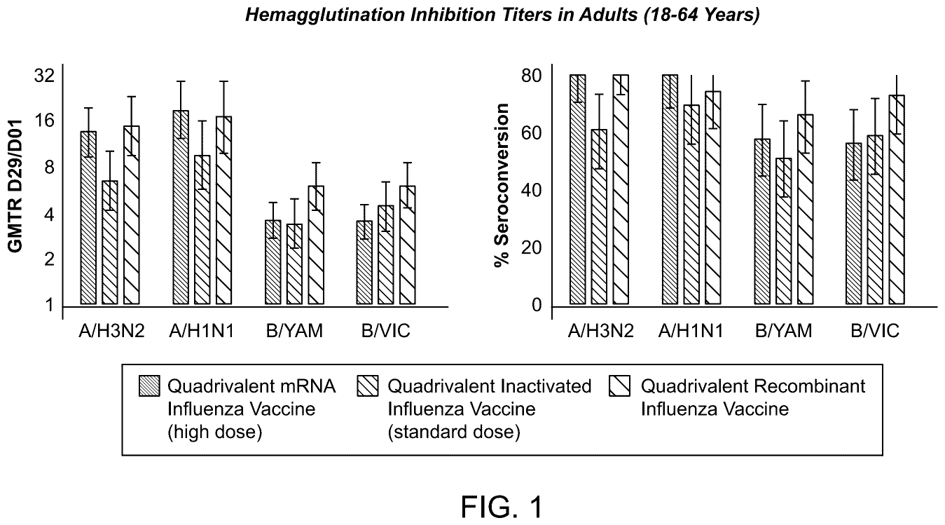

Interim results from clinical trial NCT05624606 demonstrates that administration of the 1:1:1:1 quadrivalent mRNA vaccine formulated with the cationic lipid GL-HEPES-E3-E12-DS-4-E10 generated hemagglutinin inhibition (HAI) antibodies against all four influenza strains at all three dose levels (i.e., low, medium, high) in adults 18 to 64 years of age. Further, while immune responses for influenza A strains (e.g., A/H1N1, A/H3N2) were comparable to the controls, lower immune responses were seen for influenza B strains (e.g., B/Yamagata-lineage, and B/Victoria-lineage) in adults 18 to 64 years of age, as shown in FIG. 1.

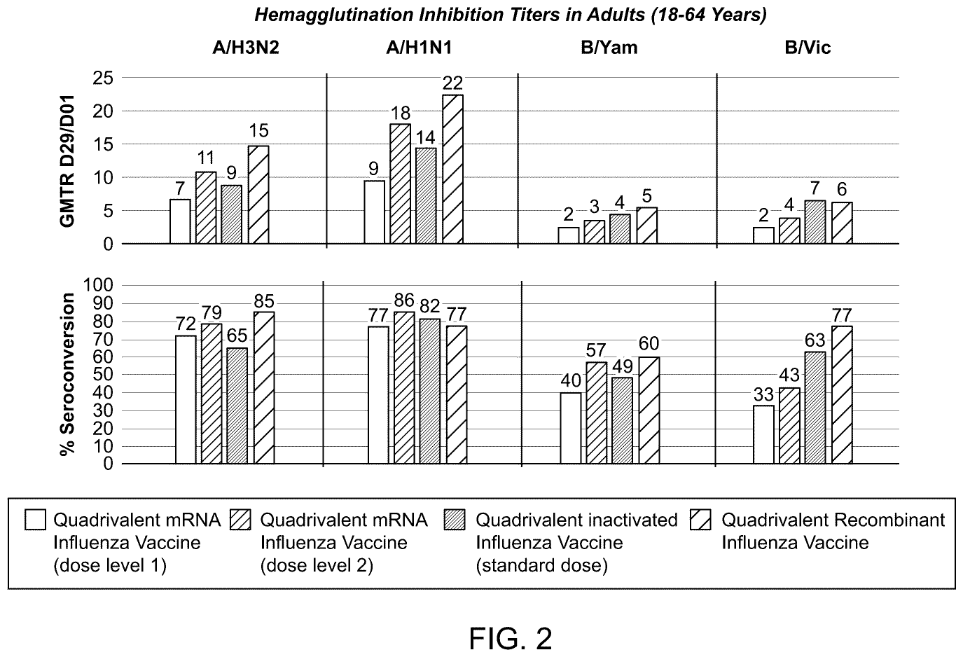

Interim results from clinical trial NCT05553301 demonstrates that administration of the 1:1:1:1 quadrivalent mRNA vaccine formulated with the cationic lipid ckk-E10 generated HAI antibodies against all four influenza strains at both dose levels (i.e., dose level 1, dose level 2) in adults 18 to 64 years of age. Further, while immune responses for influenza A strains (e.g., A/H1N1, A/H3N2) were comparable to the controls, lower immune responses were seen for influenza B strains (e.g., B/Yamagata-lineage, and B/Victoria-lineage) in adults 18 to 64 years of age, as shown in FIG. 2.

Data from the above studies demonstrate the need to improve B strain immunogenicity in combination influenza mRNA vaccine formulations.

### Example 3: Improving B Strain Immunogenicity in Combination Influenza mRNA Vaccine Formulations

One exemplary strategy to improve B strain immunogenicity in combination influenza mRNA vaccine formulations is to increase the ratio/quantity of mRNA encoding the HA sequence(s) of the influenza B virus(es) compared to the mRNA encoding the HA sequence(s) of the influenza A virus(es).

Table 2 below depicts an exemplary clinical study protocol in which eligible participants (adults 18 to 64 years of age and >65 years of age) are randomized to receive a single intramuscular injection of either: (1) a 1:1:1:1 quadrivalent mRNA vaccine encoding the HA sequences of influenza A and B strains with a total mRNA dose of 130 μg; (2) a 1:1:4:4 quadrivalent mRNA vaccine encoding the HA sequences of influenza A and B strains with a total mRNA dose of 160 μg; (3) a 1:1:2:2 quadrivalent mRNA vaccine encoding the HA sequences of influenza A and B strains with a total mRNA dose of 200 μg; (4) a 1:1:6:6 quadrivalent mRNA vaccine encoding the HA sequences of influenza A and B strains with a total mRNA dose of 224 μg; (5) a dose of Fluzone Quadrivalent® influenza vaccine (standard dose) (Sanofi Pasteur); (6) a dose of Fluzone High-Dose Quadrivalent® influenza vaccine (Sanofi Pasteur); or (7) a dose of Flublok Quadrivalent® recombinant influenza vaccine (Sanofi Pasteur).

TABLE 2
Exemplary clinical study protocol evaluating HA mRNA ratios and total
mRNA doses in quadrivalent influenza mRNA vaccines (protocol 1)

| | | | | Total Number of | |
|---|---|---|---|---|---|
| | | | | Subjects (n = 690) | |
| | | HA mRNA Dose | Total mRNA | 18-64 | 65+ |
| Group | Product | (μg)/A:A:B:B ratio | Dose (μg) | years | years |
| 1 | Quadrivalent mRNA vaccine | 32:32:32:32/1:1:1:1 | 130 | | 30 |
| | (current benchmark) | | | | |
| 2 | Quadrivalent mRNA vaccine | 16:16:64:64/1:1:4:4 | 160 | 60 | 60 |
| | with high dose B and low | | | | |
| | dose A | | | | |
| 3 | Quadrivalent mRNA vaccine | 32:32:64:64/1:1:2:2 | 200 | 60 | 60 |
| | with high dose B | | | | |
| 4 | Quadrivalent mRNA vaccine | 16:16:96:96/1:1:6:6 | 224 | 60 | 60 |
| | with higher dose B and low | | | | |
| | dose A | | | | |
| 5 | Fluzone Quadrivalent ® | 15 μg/strain | — | 60 | 60 |
| | vaccine (standard dose) | | | | |
| 6 | Fluzone High-Dose | 60 μg/strain | — | — | 60 |
| | Quadrivalent ® vaccine | | | | |
| 7 | Flublok Quadrivalent ® | 45 μg/strain | — | 60 | 60 |
| | vaccine | | | | |

The objective of this study is to evaluate the safety and immunogenicity of four quadrivalent influenza mRNA vaccines having different A:A:B:B HA mRNA ratios and total mRNA doses, administered as a single intramuscular injection in adults 18 years of age and older, compared to the following controls: a dose of Fluzone Quadrivalent® influenza vaccine (standard dose), a comparator for study participants 18-64 years of age and ≥65 years of age; a dose of Fluzone High-Dose Quadrivalent® influenza vaccine, a comparator for study participants ≥65 years of age only; and a dose of Flublok Quadrivalent® recombinant influenza vaccine, a comparator for study participants 18-64 years of age and ≥65 years of age.

Table 3 below depicts an alternative clinical study protocol in which eligible participants (adults 18 to 64 years of age and ≥65 years of age) are randomized to receive a single intramuscular injection of either: (1) a 1:1:2:2 quadrivalent mRNA vaccine encoding the HA sequences of influenza A and B strains with a total mRNA dose of 192 μg, in an LNP comprising cationic lipid GL-HEPES-E3-E12-DS-4-E10; (2) a 1:1:2:2 quadrivalent mRNA vaccine encoding the HA sequences of influenza A and B strains with a total mRNA dose of 30 μg, in an LNP comprising cationic lipid IM-01; (3) a 1:1:2:2 quadrivalent mRNA vaccine encoding the HA sequences of influenza A and B strains with a total mRNA dose of 96 μg, in an LNP comprising cationic lipid IM-01; (4) a 1:1:2:2 quadrivalent mRNA vaccine encoding the HA sequences of influenza A and B strains with a total mRNA dose of 132 μg, in an LNP comprising cationic lipid IM-01; (5) a 1:1:2:2 quadrivalent mRNA vaccine encoding the HA sequences of influenza A and B strains with a total mRNA dose of 192 μg, in an LNP comprising cationic lipid IM-01; (6) a 1:1:2:2 quadrivalent mRNA vaccine encoding the HA sequences of influenza A and B strains with a total mRNA dose of 96 μg, in an LNP comprising cationic lipid ALC-0315; (7) a 1:1:2:2 quadrivalent mRNA vaccine encoding the HA sequences of influenza A and B strains with a total mRNA dose of 192 μg, in an LNP comprising cationic lipid ALC-0315; (5) a dose of Fluzone Quadrivalent® influenza vaccine (standard dose) QIV-SD (Sanofi Pasteur); (6) a dose of Fluzone High-Dose Quadrivalent® influenza vaccine QIV-HD (Sanofi Pasteur); or (7) a dose of Flublok Quadrivalent® recombinant influenza vaccine RIV (Sanofi Pasteur).

TABLE 3
Exemplary clinical study protocol evaluating HA mRNA
ratios and total mRNA doses with different LNPs in
quadrivalent influenza mRNA vaccines - (protocol 2)

| | | | Total | | |
|---|---|---|---|---|---|
| | | | quantity | Number of | |
| | | | of | participants | |
| | | | mRNA | (n) | |
| | | | (μg) | (N = 736) | |
| | | | per | 18-64 | ≥65 |
| Cohort | Group | Group Name | dose | years | years |
| Main | 1 | QIV mRNA/GL-HEPES-E3- | 192 | 23 | 23 |
| | | E12-DS-4-E10 192 μg | | | |
| | | 32 μg for A strains, 64 μg | | | |
| | | for B strains | | | |
| | | (ratio type 1:1:2:2) | | | |
| | 2 | QIV mRNA/IM-01 30 μg | 30 | — | — |
| | | 5 μg for A strains, 10 μg for B | | | |
| | | strains | | | |
| | | (ratio type 1:1:2:2) | | | |
| | 3 | QIV mRNA/IM-01 96 μg | 96 | 46 | 46 |
| | | 16 μg for A strains, 32 μg | | | |
| | | for B strains | | | |
| | | (ratio type 1:1:2:2) | | | |
| | 4 | QIV mRNA/IM-01 132 μg | 132 | 46 | 46 |
| | | 22 μg for A strains, 44 μg | | | |
| | | for B strains | | | |
| | | (ratio type 1:1:2:2) | | | |
| | 5 | QIV mRNA/IM-01 192 μg | 192 | 46 | 46 |
| | | 32 μg for A strains, 64 μg | | | |
| | | for B strains | | | |
| | | (ratio type 1:1:2:2) | | | |
| | 6 | QIV mRNA/ALC-0315 96 μg | 96 | 46 | 46 |
| | | 16 μg for A strains, 32 μg | | | |
| | | for B strains | | | |
| | | (ratio type 1:1:2:2) | | | |
| | 7 | QIV mRNA/ALC-0315 | 192 | 46 | 46 |
| | | 192 μg 32 μg for A | | | |
| | | strains, 64 μg for B strains | | | |
| | | (ratio type 1:1:2:2) | | | |
| | 8 | QIV-SD | — | 46 | 46 |
| | 9 | QIV-HD | — | — | 46 |
| | 10 | RIV4 | — | 46 | 46 |

| | | |
|---|---|---|
| Total number of participants: | 345 | 391 |

The objective of this study is to evaluate the safety and immunogenicity of quadrivalent influenza mRNA vaccines having different A:A:B:B HA mRNA ratios and total mRNA doses, in various LNP formulations, administered as a single intramuscular injection in adults 18 years of age and older, compared to the following controls: a dose of Fluzone Quadrivalent® influenza vaccine (standard dose), a comparator for study participants 18-64 years of age and ≥65 years of age; a dose of Fluzone High-Dose Quadrivalent® influenza vaccine, a comparator for study participants ≥65 years of age only; and a dose of Flublok Quadrivalent® recombinant influenza vaccine, a comparator for study participants 18-64 years of age and ≥65 years of age.

Exemplary nucleic acid sequences encoding exemplary influenza A and influenza B constructs that can be used in the clinical study protocol are recited below:

A/Wisconsin/588/2019
(SEQ ID NO: 5)
GGACAGAUCGCCUGGAGACGCCAUCCACGCUGUUUUGACCUCCAUAGAAG
ACACCGGGACCGAUCCAGCCUCCGCGGCCGGGAACGGUGCAUUGGAACGC
GGAUUCCCCGUGCCAAGAGUGACUCACCGUCCUUGACACGAUGAAAGCCA
UCCUUGUUGUCAUGCUGUACACAUUCACCACCGCAAAUGCGGAUACCCUG
UGUAUCGGCUACCACGCAAAUAAUUCCACCGACACCGUUGAUACCGUCCU
GGAAAAGAACGUGACAGUGACUCACAGCGUCAAUCUCCUUGAGGAUAAAC
AUAAUGGCAAGCUGUGCAAGCUGAGAGGCGUGGCUCCCCUGCAUCUGGGA
AAGUGCAACAUCGCUGGUUGGAUCCUCGGGAACCCAGAGUGUGAGUCCCU
CUCAACCGCACGGUCUUGGUCAUACAUCGUGGAGACUAGCAAUUCAGACA
ACGGCACAUGCUACCCCGGUGACUUCAUUAACUACGAGGAGCUGAGAGAA
CAGCUGAGUUCCGUGUCAUCCUUCGAGAGAUUCGAAAUCUUCCCCAAAAC
CUCCUCCUGGCCCAAUCAUGACUCCGACAAUGGAGUGACAGCCGCUUGUC
CCCACGCCGGUGCCAAGAGUUUCUAUAAGAACCUCAUCUGGCUGGUGAAA
AAGGGCAAGUCCUAUCCCAAAAUUAACCAGACCUACAUUAACGAUAAGGG
GAAAGAAGUCCUGGUCCUGUGGGGGAUACACCACCCCCCUACCAUCGCCG
ACCAGCAGUCUCUGUAUCAGAACGCCGACGCCUACGUGUUCGUGGGUACC
AGCCGUUAUAGUAAAAAGUUCAAGCCAGAAAUUGCCACCAGACCUAAGGU
GCGCGACCAGGAGGGCCGCAUGAACUACUACUGGACCCUGGUGGAACCUG
GCGACAAGAUUACAUUCGAGGCCACUGGGAACCUGGUGGCACCCAGAUAC
GCCUUUACAAUGGAACGGGAUGCUGGGAGCGGAAUCAUUAUCUCCGAUAC
CCCUGUCCACGACUGCAAUACUACCUGUCAGACCCCAGAAGGCGCUAUCA
AUACCUCUCUGCCUUUCCAAAACGUGCACCCUAUCACUAUCGGGAAAUGU
CCCAAGUAUGUGAAAAGCACCAAACUGCGCCUGGCAACCGGUCUGAGAAA
UGUGCCCUCCAUCCAGUCCCGCGGCUUGUUCGGUGCAAUCGCUGGCUUUA
UCGAGGGUGGCUGGACUGGAAUGGUCGAUGGCUGGUACGGCUACCAUCAC
CAGAACGAGCAGGGGUCCGGGUAUGCUGCCGACCUGAAAAGCACUCAGAA
CGCCAUCGAUAAAAUCACUAACAAGGUGAACUCCGUGAUCGAAAAGAUGA
AUACACAGUUCACAGCAGUUGGCAAGGAGUUCAACCACCUGGAAAAACGG
AUAGAGAACCUGAAUAAGAAAGUCGAUGAUGGCUUUCUGGACAUCUGGAC
UUACAAUGCCGAGCUGCUGGUGCUCCUGGAAAACGAGCGGACACUGGAUU
AUCACGACUCAAACGUGAAGAACCUGUAUGAAAAGGUGCGUAACCAGCUG
AAAAACAACGCCAAGGAAAUCGGCAAUGGCUGUUUCGAAUUUUACCACAA
GUGUGAUAAUACCUGUAUGGAGAGCGUUAAGAACGGGACUUACGACUACC
CAAAAUACAGCGAGGAGGCCAAGCUGAACCGGGAGAAGAUCGACGGCGUC
AAACUCGACUCCACUAGAAUAUACCAGAUUCUCGCCAUCUAUAGCACAGU
GGCAUCAAGUCUCGUCCUGGUGGUGUCACUGGGAGCCAUCAGCUUUUGGA
UGUGCAGCAAUGGAUCCCUCCAGUGUAGGAUCUGCAUCUAACGGGUGGCA
UCCCUGUGACCCCUCCCCAGUGCCUCUCCUGGCCCUGGAAGUUGCCACUC
CAGUGCCCACCAGCCUUGUCCUAAUAAAAUUAAGUUGCAUC.
A/Tasmania/503/2020
(SEQ ID NO: 6)
GGACAGAUCGCCUGGAGACGCCAUCCACGCUGUUUUGACCUCCAUAGAAG
ACACCGGGACCGAUCCAGCCUCCGCGGCCGGGAACGGUGCAUUGGAACGC
GGAUUCCCCGUGCCAAGAGUGACUCACCGUCCUUGACACGAUGAAGACCA
UCAUCGCUCUGUCCUACAUCCUGUGCCUGGUGUUUGCUCAGAAAAUCCCC
GGGAAUGACAAUUCCACUGCCACUCUCUGCCUGGGCCAUCAUGCCGUGCC
AAAUGGAACCAUUGUCAAGACUAUAACAAAUGACCGCAUCGAAGUGACCA
ACGCUACCGAGCUGGUUCAGAACAGCAGUAUUGGAGAAAUCUGCGAUUCC
CCACACCAGAUACUGGAUGGCGGCAACUGCACCCUGAUCGACGCACUGCU
GGGUGACCCUCAGUGCGACGGAUUUCAGAAUAAGGAGUGGGACCUUUUCG
UUGAGCGCAGCAGAGCCAAUAGCAACUGCUACCCGUACGACGUGCCGGAU
UACGCCAGUCUUCGAAGCCUGGUCGCAUCCAGCGGGACACUGGAGUUUAA
GAAUGAGUCCUUUAAUUGGACAGGCGUGAAGCAGAACGGGACUAGCAGCG
CAUGCAUUCGGGGCAGUAGCUCAUCCUUCUUUAGCCGACUGAACUGGCUG
ACCCACCUCAACUACACAUACCCCGCACUGAAUGUGACUAUGCCAAACAA
AGAACAGUUUGACAAACUGUACAUCUGGGGAGUGCACCAUCCUAGCACAG
ACAAGGACCAGAUCAGCCUGUUUGCCCAGCCCAGCGGCAGGAUUACCGUG
UCCACAAAACGGUCACAGCAAGCCGUGAUCCCUAAUAUUGGAUCCCGCCC
CCGGAUAAGGGACAUCCCUAGUCGCAUCAGUAUCUACUGGACCAUCGUGA
AGCCCGGAGAUAUCUUGCUCAUCAAUAGCACUGGCAACCUCAUUGCCCCC
AGGGGCUAUUUUAAGAUCAGAAGCGGCAAGUCCAGCAUUAUGCGCAGCGA
CGCACCCAUUGGCAAGUGCAAGUCCGAGUGCAUCACUCCUAAUGGGUCCA
UCCCAAACGACAAGCCAUUCCAAAAUGUCAACAGAAUCACCUACGGGGCU
UGCCCCCGCUACGUGAAGCAGAGUACACUGAAACUGGCCACCGGGAUGCG
CAACGUGCCCGAGAAGCAAACUAGAGGCAUCUUUGGAGCUAUCGCUGGCU
UCAUUGAGAAUGGCUGGGAGGGUAUGGUGGACGGCUGGUACGGAUUCCGC
CACCAGAAUAGCGAAGGCAGAGGCCAGGCAGCAGACUUGAAGUCCACCCA
GGCCGCCAUUGAUCAGAUCAACGGCAAACUGAAUCGGCUUAUUGGAAAAA
CAAACGAGAAGUUCCAUCAGAUUGAGAAGGAGUUUAGCGAGGUGGAGGGC
CGCGUGCAGGAUCUGGAAAAGUACGUUGAAGACACCAAGAUCGACCUGUG
GUCAUACAAUGCAGAGCUGCUCGUUGCCCUGGAAAAUCAGCACACAAUUG
ACCUUACAGACUCCGAAAUGAAUAAGCUCUUUGAAAAGACCAAGAAGCAG
CUGCGCGAGAACGCCGAGGAUAUGGGGAACGGUUGUUUUAAGAUCUACCA
CAAGUGUGACAACGCCUGCAUUGGGUCCAUCCGAAAUGAAACAUACGACC
ACAACGUGUAUAGAGAUGAGGCCCUGAACAACCGAUUCCAGAUUAAGGGA
GUCGAGCUGAAGAGUGGCUAUAAGGACUGGAUCCUGUGGAUCUCAUUCGC
CAUGUCAUGCUUCCUUCUGUGUAUUGCUCUGCUCGGCUUCAUCAUGUGGG
CUUGCCAGAAAGGCAAUAUCCGGUGCAACAUCUGCAUCUAACGGGUGGCA
UCCCUGUGACCCCUCCCCAGUGCCUCUCCUGGCCCUGGAAGUUGCCACUC
CAGUGCCCACCAGCCUUGUCCUAAUAAAAUUAAGUUGCAUC.
B/Washington/02/2019
(SEQ ID NO: 7)
GGACAGAUCGCCUGGAGACGCCAUCCACGCUGUUUUGACCUCCAUAGAAG
ACACCGGGACCGAUCCAGCCUCCGCGGCCGGGAACGGUGCAUUGGAACGC
GGAUUCCCCGUGCCAAGAGUGACUCACCGUCCUUGACACGAUGAAAGCAA
UCAUAGUGCUGCUGAUGGUGGUGACUAGCAAUGCCGAUCGGAUCUGCACC
GGCAUCACUUCCAGUAACAGCCCUCAUGUGGUCAAAACCGCCACACAGGG
CGAGGUGAACGUGACCGGAGUGAUUCCACUGACAACUACACCAACGAAGA
GUCACUUCGCCAACCUGAAGGGCACCGAAACACGAGGCAAGCUCUGCCCC
AAGUGUCUGAAUUGCACCGACCUGGACGUCGCUUUGGGCCGCCCUAAAUG
UACCGGCAAAAUACCUUCCGCCAGAGUGUCCAUCCUGCACGAGGUGCGCC
CCGUGACCUCCGGGUGUUUUCCCAUAAUGCACGACCGCACUAAAAUCCGC
CAGCUGCCCAAUCUUCUGAGGGGGUACGAACAUGUCAGGCUGUCCACUCA
CAACGUGAUCAACGCAGAAGACGCCCCCGGAAGGCCUUAUGAGAUUGGAA
CCAGUGGGUCCUGCCCAAACAUUACCAACGGCAACGGCUUCUUCGCCACU
AUGGCCUGGGCCGUGCCAAAGAACAAGACCGCCACCAACCCCCUGACAAU
UGAAGUCCCUUACAUCUGCACAGAGGGAGAGGAUCAGAUCACCGUGUGGG
GGUUUCACUCUGAUAACGAAACUCAGAUGGCCAAGCUGUACGGGGAUUCU
AAACCCCAGAAGUUCACCAGUAGCGCUAACGGGGUGACCACCCAUUAUGU
GUCUCAGAUCGGAGGUUUCCCAAAUCAGACCGAGGACGGCGGACUGCCCC
AGUCUGGAAGGAUCGUAGUGGACUAUAUGGUGCAGAAGAGUGGAAAAACC
GGCACCAUUACCUAUCAGCGCGGCAUACUGCUGCCACAGAAGGUGUGGUG
UGCUUCCGGCAGGUCCAAGGUUAUCAAAGGGUCCCUCCCCCUGAUCGGCG
AAGCAGAUUGUCUGCACGAGAAGUACGGCGGACUGAAUAAGAGCAAACCC
UACUACACCGGAGAACACGCUAAGGCAAUUGGGAAUUGUCCGAUCUGGGU
GAAGACGCCCCUGAAACUGGCCAAUGGCACAAAAUACCGGCCCCCCGCUA
AGCUGCUGAAGGAACGGGGGUUCUUCGGCGCCAUAGCCGGCUUUCUGGAG
GGAGGCUGGGAGGGCAUGAUAGCCGGGUGGCACGGCUACACUUCCCAUGG
GGCUCACGGGGUGGCUGUGGCCGCCGACCUGAAGUCUACGCAGGAAGCUA
UCAACAAAAUCACUAAGAACCUGAACAGCCUGUCGGAAUUGGAGGUCAAG
AAUCUGCAGCGGCUGAGCGGCGCCAUGGAUGAGCUGCACAAUGAGAUCCU
GGAGCUUGACGAGAAGGUCGAUGAUCUUCGGGCCGAUACAAUUAGUAGCC
AAAUUGAGUUGGCCGUGCUGCUCAGCAACGAAGGCAUAAUCAACAGCGAG
GACGAGCACCUCCUGGCUCUGGAGAGAAAGCUGAAGAAGAUGCUCGGCCC
UAGCGCAGUUGAGAUCGGAAACGGCUGCUUCGAAACCAAGCACAAGUGCA
ACCAGACCUGCCUGGACAGGAUCGCGGCAGGAACAUUCGACGCUGGGGAA
UUCAGCCUCCCCACCUUCGACAGCCUGAACAUCACAGCCGCCAGUCUGAA
UGAUGACGGACUGGAUAACCAUACCAUCCUGCUGUACUACUCUACCGCUG
CUUCCUCCCUGGCCGUGACAUUGAUGAUCGCAAUCUUUGUGGUUUAUAUG
GUGAGCCGAGACAACGUCAGUUGCAGUAUCUGCCUUUAACGGGUGGCAUC
CCUGUGACCCCUCCCCAGUGCCUCUCCUGGCCCUGGAAGUUGCCACUCCA
GUGCCCACCAGCCUUGUCCUAAUAAAAUUAAGUUGCAUC.
B/Phuket/3073/2013
(SEQ ID NO: 8)
GGACAGAUCGCCUGGAGACGCCAUCCACGCUGUUUUGACCUCCAUAGAAG
ACACCGGGACCGAUCCAGCCUCCGCGGCCGGGAACGGUGCAUUGGAACGC
GGAUUCCCCGUGCCAAGAGUGACUCACCGUCCUUGACACGAUGAAAGCCA
UCAUUGUGCUGCUGAUGGUUGUUACAAGCAACGCCGACCGCAUCUGCACC
GGGAUUACAAGCAGCAAUAGCCCUCACGUGGUGAAGACAGCAACACAGGG
AGAGGUGAACGUGACCGGCGUGAUUCCACUGACAACCACCCCAACUAAAU
CUUACUUUGCAAACCUGAAAGGGACACGGACCAGAGGAAAGCUGUGCCCU
GAUUGCCUGAAUUGCACAGACCUGGACGUGGCCCUGGGCAGACCAAUGUG
CGUGGGCACUACACCAAGCGCCAAGGCCUCCAUCCUCCAUGAGGUGCGGC
CCGUGACUUCUGGAUGUUUCCCCAUUAUGCACGACAGAACCAAGAUUAGA
CAGCUGCCAAACCUGCUCCGCGGCUACGAGAAAAUUCGCCUGUCUACACA
GAAUGUGAUCGACGCCGAGAAGGCUCCAGGAGGACCAUACAGACUGGGGA
CUUCUGGCAGCUGCCCUAACGCCACCUCUAAGAUCGGGUUCUUCGCAACC
AUGGCUUGGGCCGUGCCUAAAGACAAUUACAAGAAUGCCACCAAUCCACU
GACUGUCGAGGUGCCAUAUAUUUGCACAGAGGGGGAGGACCAGAUCACUG
UGUGGGGCUUUCAUAGCGAUAAUAAGACUCAGAUGAAGUCUCUCUACGGC
GACUCUAACCCUCAGAAGUUCACCUCCUCUGCCAACGGGGUGACAACACA
CUACGUGUCCCAGAUCGGGGACUUUCCUGACCAGACCGAGGAUGGAGGAC
UGCCUCAGUCUGGACGCAUCGUGGUGGACUAUAUGAUGCAGAAGCCUGGG
AAGACCGGCACUAUCGUGUACCAGAGGGGCGUGCUGCUGCCCCAAAAGGU
GUGGUGUGCCUCCGGAAGAAGCAAAGUGAUUAAGGGAUCCCUGCCUCUGA
UUGGGGAGGCCGAUUGCCUGCAUGAAGAGUAUGGAGGGCUGAACAAGUCC
AAGCCAUACUAUACAGGAAAGCACGCAAAAGCCAUCGGCAACUGUCCCAU
CUGGGUCAAAACUCCUCUGAAGCUGGCCAACGGCACCAAAUACCGCCCUC
CAGCCAAGCUGCUGAAAGAACGCGGAUUCUUCGGCGCCAUUGCAGGGUUU
CUGGAAGGAGGCUGGGAGGGCAUGAUUGCUGGAUGGCACGGAUAUACCUC
UCACGGCGCUCACGGGGUGGCCGUGGCCGCCGAUCUGAAGUCCACACAGG
AGGCAAUUAACAAGAUCACCAAGAAUCUGAAUUCACUGUCCGAGCUCGAA
GUGAAAAACCUGCAGCGCCUGUCCGGCGCCAUGGACGAGCUGCACAAUGA
AAUCCUGGAGCUGGACGAGAAGGUGGACGACCUGCGGGCUGACACUAUCA
GCAGCCAGAUCGAGCUGGCAGUGCUGCUGAGCAAUGAGGGCAUCAUCAAC
UCAGAAGACGAACACCUCCUGGCACUGGAAAGGAAACUCAAGAAGAUGCU
GGGCCCCUCCGCAGUGGACAUUGGGAACGGCUGUUUCGAAACCAAGCAUA
AGUGUAACCAGACUUGUCUGGAUAGGAUCGCAGCAGGAACCUUCAACGCC
GGCGAAUUUUCUCUGCCAACAUUUGACUCCCUGAACAUCACAGCUGCAUC
CCUGAACGACGACGGACUGGACAAUCACACCAUCCUGCUGUACUACUCUA
CUGCCGCUAGCUCCCUGGCCGUGACCCUGAUGCUGGCCAUCUUCAUCGUG
UACAUGGUUUCCAGGGAUAACGUGUCUUGUAGCAUUUGCCUGUAACGGGU
GGCAUCCCUGUGACCCCUCCCCAGUGCCUCUCCUGGCCCUGGAAGUUGCC
ACUCCAGUGCCCACCAGCCUUGUCCUAAUAAAAUUAAGUUGCAUC.

### Example 4: Use of IM-001 Containing LNPs to Induce HA Immune Response

LNPs comprising IM-001 at a molar ratio of 40%; DMG-PEG2000 at a molar ratio of 1.5%; cholesterol at a molar ratio of 28.5%; and DOPE at a molar ratio of 30% were used for delivery of HA antigen in mice. Groups of Balb/c mice (Mus musculus) as per the treatment group were immunized under isoflurane anesthesia with a dose of 0.4 μg per mouse in 0.05 mL of IM-001/Modified Tasmania H3 mRNA-lipid nanoparticles via the IM route in the quadriceps, on day 0 in one hind leg and day 21 in the contralateral leg. Mice were evaluated for a minimum of 3 days post-administration and any animal that lost displayed severe clinical signs after the veterinarian's assessment was euthanized by administration of 5 mg/kg of meloxicam by subcutaneous injection.

Blood was collected via submandibular or orbital sinus bleeds (in-life bleeds were performed on day-1 and on day 20) and cardiac puncture (terminal bleed, day 35) from all animals under sedation. Mice were bled on pre-study to obtain a base-line pre-immune serum sample and for pre-screening purposes.

HAI assays were performed using the A/Tasmania/503/2020 (H3N2) virus stocks (BIOQUAL, Inc.). Sera were treated with receptor-destroying enzyme (RDE) by diluting one-part serum with three parts enzyme and incubated overnight in a 37° C. water bath. Enzyme was inactivated by a 30-minute incubation period at 56° C. followed by addition of six parts PBS for a final dilution of 1/10. HAI assays were performed in V-bottom 96-well plates using four hemagglutinating units (HAU) of virus and 0.5% turkey RBC. The reference serum for each strain was included as a positive control on every assay plate. Each plate also included a back-titration to confirm the antigen dose (4 HAU/25 pl) as well as a negative control sample (PBS or naive control serum). The HAI titer was determined as the highest dilution of serum resulting in complete inhibition of hemagglutination. Results were only valid for plates with the appropriate back-titration result (verifying 4 HAU/25 μl added) and a reference serum titer within 2-fold of the expected titer. The vaccine was shown to induce an HAI GMT of 320, demonstrating high levels of induction of immunogenicity against influenza antigens with these LNPs.

1. A composition comprising at least three messenger RNAs (mRNAs), wherein the at least three mRNAs comprise an open reading frame (ORF) encoding a hemagglutinin (HA) antigen selected from the group consisting of:
(i) a first mRNA encoding an HA antigen of a first influenza A virus;
(ii) a second mRNA encoding an HA antigen of a second influenza A virus, wherein the first influenza A virus and the second influenza A virus are of different subtypes; and
(iii) a third mRNA encoding an HA antigen of a first influenza B virus,
wherein the mRNA encoding the HA antigen of the influenza A virus is present in a different ratio (w/w) than the mRNA encoding the HA antigen of the influenza B virus.

2. The composition of claim 1, further comprising a fourth mRNA encoding an HA antigen of a second influenza B virus, and wherein the first influenza B virus and the second influenza B virus are of different lineages.

3. The composition of claim 1, wherein the first mRNA, the second mRNA, and the third mRNA are present in the ratio (w/w) of about 1:1:2, about 1:1:3, about 1:1:4, about 1:1:5, about 1:1:6, about 1:1:7, about 1:1:8, about 1:1:9 or about 1:1:10.

4. The composition of claim 2, wherein the first mRNA, the second mRNA, the third mRNA, and the fourth mRNA are present in the ratio (w/w) of about 1:1:2:2, about 1:1:3:3, about 1:1:4:4, about 1:1:5:5, about 1:1:6:6, about 1:1:7:7, about 1:1:8:8, about 1:1:9:9 or about 1:1:10:10.

5. The composition of claim 1, wherein the ratio is expressed in micrograms (μg).

6. The composition of claim 1, wherein the first mRNA, the second mRNA, the third mRNA, and/or the fourth mRNA are formulated into a LNP.

7. The composition of claim 6, wherein the LNP comprises at least one cationic lipid, optionally selected from the group consisting of OF-02, cKK-E10, GL-HEPES-E3-E10-DS-3-E18-1, GL-HEPES-E3-E12-DS-4-E10, GL-HEPES-E3-E12-DS-3-E14, (4-hydroxybutyl)azanediyl]di(hexane-6,1-diyl) bis(2-hexyldecanoate) (ALC-0315) and IM-001.

8. The composition of claim 6, wherein the LNP further comprises a polyethylene glycol (PEG) conjugated (PEGylated) lipid, a cholesterol-based lipid, and a helper lipid.

9. The composition of claim 6, wherein the LNP comprises:
a cationic lipid at a molar ratio of 35% to 55%;
a polyethylene glycol (PEG) conjugated (PEGylated) lipid at a molar ratio of 0.25% to 2.75%;
a cholesterol-based lipid at a molar ratio of 20% to 45%; and
a helper lipid at a molar ratio of 5% to 35%,
wherein all of the molar ratios are relative to the total lipid content of the LNP;
optionally wherein the LNP comprises:
a cationic lipid at a molar ratio of 40%;
a PEGylated lipid at a molar ratio of 1.5%;
a cholesterol-based lipid at a molar ratio of 28.5%; and
a helper lipid at a molar ratio of 30%,
wherein all of the molar ratios are relative to the total lipid content of the LNP.

10. The composition of claim 8, wherein the PEGylated lipid is dimyristoyl-PEG2000 (DMG-PEG2000) or 2-[(polyethylene glycol)-2000]-N,N-ditetradecylacetamide (ALC-0159); and/or the cholesterol-based lipid is cholesterol; and/or the helper lipid is 1,2-dioleoyl-SN-glycero-3-phosphoethanolamine (DOPE) or 1,2-distearoyl-sn-glycero-3-phosphocholine (DSPC).

11. The composition of claim 1, wherein the first mRNA encodes an HA antigen of the influenza A subtype H1N1, and/or the second mRNA encodes an HA antigen of the influenza A subtype H3N2, and/or the third mRNA encodes an HA antigen of the influenza B Victoria-lineage strain.

12. The composition of claim 2, wherein the fourth mRNA encodes an HA antigen of the influenza B Yamagata-lineage strain.

13. A composition comprising at least three messenger RNAs (mRNAs), wherein:
(i) a first mRNA encodes a hemagglutinin (HA) antigen of a first influenza A virus;
(ii) a second mRNA encodes an HA antigen of a second influenza A virus, wherein the first influenza A virus and the second influenza A virus are of different subtypes; and
(iii) a third mRNA encodes an HA antigen of a first influenza B virus,
wherein the first mRNA, the second mRNA, and the third mRNA are formulated into a lipid nanoparticle (LNP) comprising IM-001.

14. A method of eliciting an immune response to influenza A or protecting a subject against influenza A infection, comprising administering to the subject the composition of claim 1.

15. A method of eliciting an immune response to influenza B or protecting a subject against influenza B infection, comprising administering to the subject the composition of claim 1.

16. The composition of claim 2, wherein the first mRNA, the second mRNA, the third mRNA, and the fourth mRNA are present in a ratio (w/w) of about 32 micrograms of the first mRNA, to about 32 micrograms of the second mRNA, to about 64 micrograms of the third mRNA, to about 64 micrograms of the fourth mRNA.

17. The composition of claim 2, wherein the first mRNA, the second mRNA, the third mRNA, and the fourth mRNA are present in a ratio (w/w) of about 16 micrograms of the first mRNA, to about 16 micrograms of the second mRNA, to about 64 micrograms of the third mRNA, to about 64 micrograms of the fourth mRNA.

18. The composition of claim 2, wherein the first mRNA, the second mRNA, the third mRNA, and the fourth mRNA are present in a ratio (w/w) of about 16 micrograms of the first mRNA, to about 16 micrograms of the second mRNA, to about 96 micrograms of the third mRNA, to about 96 micrograms of the fourth mRNA.

19. The composition of claim 2, wherein the composition comprises 130 micrograms of the mRNA in total, 160 micrograms of the mRNA in total, 200 micrograms of the mRNA in total, or 224 micrograms of the mRNA in total.
